SEQUENCE LISTING (71 sequences)

SEQ ID NO: 1 - protein, 220 aa, Homo sapiens
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 220 aa, Homo sapiens
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 220 aa, synthetic construct
  UNSURE 67: note = E or K
  UNSURE 28: note = R or Q
  UNSURE 84: note = Q or L
  UNSURE 41: note = H or L
  UNSURE 129: note = D or E
  UNSURE 131: note = L or M
  UNSURE 107: note = K or E
  UNSURE 110: note = S or F
PCPAPELLGGPSVFLFPPKPKDTLMISXTPEVTCVVVDVSXEDPEVKFNWYVDGVEVHNAKTKPREXQYNSTYRVVSVLTVLHXDWLNGKEYKCKVSNKALPAPIEXTIXKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 221 aa, synthetic construct
  UNSURE 129: note = D or E
  UNSURE 131: note = L or M
PCPAPELLGGPSVFLFPPKPKDTLMISQTPEVTCVVVDVSLEDPEVKFNWYVDGVEVHNAKTKPREKQYNSTYRVVSVLTVLHLDWLNGKEYKCKVSNKALPAPIEETISKAKGQPREPQVYTLPPSRXDXLTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 220 aa, synthetic construct
  UNSURE 129: note = D or E
  UNSURE 131: note = L or M
PCPAPELLGGPSVFLFPPKPKDTLMISQTPEVTCVVVDVSHEDPEVKFNWYVDGVEVHNAKTKPREEQYNSTYRVVSVLTVLHQDWLNGKEYKCKVSNKALPAPIEKTIFKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 232 aa, synthetic construct
EPKSCDKTHTCPPCPAPELLGGPSVFLFPPKPKDTLMISQTPEVTCVVVDVSLEDPEVKFNWYVDGVEVHNAKTKPREKQYNSTYRVVSVLTVLHLDWLNGKEYKCKVSNKALPAPIEETISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 232 aa, synthetic construct
EPKSCDKTHTCPPCPAPELLGGPSVFLFPPKPKDTLMISQTPEVTCVVVDVSHEDPEVKFNWYVDGVEVHNAKTKPREEQYNSTYRVVSVLTVLHQDWLNGKEYKCKVSNKALPAPIEKTIFKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 107 aa, Homo sapiens [sequence omitted]

SEQ ID NO: 10 - protein, 105 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTEC

SEQ ID NO: 11 - protein, 234 aa, Human betaherpesvirus 5
MQTYSTPLTLVIVTSLFLFTTQGSSSNAVEPTKKPLKLANYRATCEDRTRTLVTRLNTSHHSVVWQRYDIYSRYMRRMPPLCIITDAYKETTRQGGAAFACTRQNLTLYNLTVKDTGVYLLQDQYTGDVEAFYLIIHPRSFCRALETRRCFYPGPGRVVVTDSQEADRAIISDLKRQWSGLSLHCAWVSGMMIFVGALVICFLRSQRIGEQDAEHLRTDLDTEPLLLTVDGDLQ

SEQ ID NO: 12 - protein, 351 aa, Human betaherpesvirus 5
MCPALAIALAAALLSNTHPGMGSSTTSAVTSPNITVTFTTSINTPNNVTSTVTTTVQTSTFTSGSTSTSASTSVATTTQKEGHLYNVNCEASYSYDQVSLNATCKVNLLNNTKNPDILSVTCYARTNCKGPFTQVGYLSAFPSDDKGKLHLSYNATAQELLISGLRPQETTEYTCSFFSWGRHHNATWDLFTYPIYAVYGTRLNATTMRVRVLLQEHEHCLLNGSSLYHPNSTVHLHQGNQLIPPWNISNVTYNGQRLREFVFYLNGTYTVVRLHVQIAGRSFTTTYVFIKSDPLFEDRLLAYGVLAFLVFMVIILLYVTYMLARRRDWSYKRLEEPVEEKKHPVPYFKQW

SEQ ID NO: 13 - protein, 293 aa, Human betaherpesvirus 5
MHWHLAITWTVIILTFSECYNQTCPCPCICVNSTTVSISTSETTSKNITPTTTTNSKKTTSSIATTTPSLVTTGKVVSTAASSTIISQTNRSHTSNAITTPKTRFEYNITGYVGQEVTLNFTGSWNYIQWFRYGSPGWLYSSEPICTVTSNYHHTFPRGALCFDCDMTKLLIYDLTLNDSGKYVVKRTRHDNQYEEACYSLTVIFANTTSIVTNRTCDRKRTENTDTTNHEIGKHIIETIKKANIPLGIHAVWAGIVVSVALIALYMGNRRRPRKPRYTRLPKYDPDESWTKT

SEQ ID NO: 14 - protein, 416 aa, Human betaherpesvirus 5
MRVACRRPHHLTYRHTAYTIIIFYILHRVTCNSTTTNTASITSPNTASTTFVTSVFSTPNNNTSTTPHTSVTSQASTIGNITNVTSDLSTFTTVYSTFNTSYANISNTAATTELISTNTNTILSFTNVTANATSSYNTTITVTITSDETSHNVSTNTALISTPWLTNCSATTYTTYNRTNSSNACHTETTIIRFKETNTTGIEGSNVTIKGNSTWDCLSVAWIRHYNRSTHGHHLGHRKNAHTQSWYWLRILTSHTVCHSQHERPSLYHDLCRSCNNTELHLYDLNITNSGRYSRRCFKENYFTGHHEDENFYLLVTPKNHTEAINATFVCPRYNTDIENEDREKGSQHTNNTHHHKRNLYHSSQRSRTVWTIVLVCMACIVLFFARRAFNKKYHMLQDTVSESEFIVRYHTEHED

SEQ ID NO: 15 - protein, 414 aa, Human betaherpesvirus 5
MRVDRQKRNNFTYRQTVYIILTFYIVYRGKCNSTDTNNSTSTSDSTVSDTSVYSTPNLPSTFFTTLDTSTGSQISTTSNTISSTTNTLAASSITTLNTSTNTTSYTASTLTALSSTHTNSPILSNNATLYTTSLDNTTTDITSSESSINASTIHNTTYSPVTSIAVNCTVAANETNNSSSKNCQQDIGTIRVKSTTLTAEEGKNITIQGNSTWNCPNVVWYRHYNWSTHGHHIYPNRRYQTPTYQHKILTSHPICHPDVSSPAAYHDLCRSCNKTELRIYDLNTTNSGRYSRRCYKEYNHDGPHEDENFGLTVNPRNDTDNHTTPLCPRYVGTQSEEDEDDDYTLSTITNNNMRKTSHRDISHGTRTTWALTLICIACILLFFVRRALNKRYRPLRDDISESSFVVRYHPEHED

SEQ ID NO: 16 - protein, 159 aa, Human betaherpesvirus 5
SSSNAVEPTKKPLKLANYRATCEDRTRTLVTRLNTSHHSVVWQRYDIYSRYMRRMPPLCIITDAYKETTRQGGAAFACTRQNLTLYNLTVKDTGVYLLQDQYTGDVEAFYLIIHPRSFCRALETRRSFYPGPGRVVVTDSQEADRAIISDLKRQWSGLS

SEQ ID NO: 17 - DNA, 27 bp, synthetic construct
  source 1..27: mol_type = other DNA
ggtggttctgctagcgacaaaactcac

SEQ ID NO: 18 - DNA, 38 bp, synthetic construct
  source 1..38: mol_type = other DNA
ttatcagatctcgagctattatttacccggagacaggg

SEQ ID NO: 19 - DNA, 27 bp, synthetic construct
  source 1..27: mol_type = other DNA
ggtggttctgctagcgacaaaactcac

SEQ ID NO: 20 - DNA, 30 bp, synthetic construct
  source 1..30: mol_type = other DNA
ggtgtacacctgtggttctcggggctgccc

SEQ ID NO: 21 - DNA, 82 bp, synthetic construct
  source 1..82: mol_type = other DNA
acgacgaagaggacgaggacgacaacgtctgataaggaaggcgagaacgtgttttgcaccccagtgaattcgagctcggtac

SEQ ID NO: 22 - DNA, 82 bp, synthetic construct
  source 1..82: mol_type = other DNA
tgtatacgccgtatgcctgtacgtgagatggtgaggtcttcggcaggcgacacgcatcttgaccatgattacgccaagctcc

SEQ ID NO: 23 - DNA, 82 bp, synthetic construct
  source 1..82: mol_type = other DNA
cggacggacctagatacggaacctttgttgttgacggtggacggggatttacagtaaaagccagtgaattcgagctcggtac

SEQ ID NO: 24 - DNA, 82 bp, synthetic construct
  source 1..82: mol_type = other DNA
ccttacagaatgttttagtttattgttcagcttcataagatgtctgcccggaaacgtagcgaccatgattacgccaagctcc

SEQ ID NO: 25 - DNA, 82 bp, synthetic construct
  source 1..82: mol_type = other DNA
ttgtttattttgttggcaggttggcgggggaggaaaaggggttgaacagaaaggtaggtgccagtgaattcgagctcggtac

SEQ ID NO: 26 - DNA, 82 bp, synthetic construct
  source 1..82: mol_type = other DNA
aggtgacgcgacctcctgccacatatagctcgtccacacgccgtctcgtcacacggcaacgaccatgattacgccaagctcc

SEQ ID NO: 27 - protein, 125 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 28 - protein, 221 aa, synthetic construct
  UNSURE 129: note = D or E
  UNSURE 131: note = L or M
PCPAPELLGGPSVFLFPPKPKDTLMISQTPEVTCVVVDVSLEDPEVKFNWYVDGVEVHNAKTKPREEQYNSTYRVVSVLTVLHLDWLNGKEYKCKVSNKALPAPIEETISKAKGQPREPQVYTLPPSRXDXLTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 232 aa, synthetic construct
EPKSCDKTHTCPPCPAPELLGGPSVFLFPPKPKDTLMISQTPEVTCVVVDVSLEDPEVKFNWYVDGVEVHNAKTKPREEQYNSTYRVVSVLTVLHLDWLNGKEYKCKVSNKALPAPIEETISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 221 aa, synthetic construct
  UNSURE 129: note = D or E
  UNSURE 131: note = L or M
PCPAPELLGGPSVFLFPPKPKDTLMISQTPEVTCVVVDVSLEDPEVKFNWYVDGVEVHNAKTKPREEQYNSTYRVVSVLTVLHLDWLNGKEYKCKVSNKALPAPIEETIFKAKGQPREPQVYTLPPSRXDXLTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 232 aa, synthetic construct
EPKSCDKTHTCPPCPAPELLGGPSVFLFPPKPKDTLMISQTPEVTCVVVDVSLEDPEVKFNWYVDGVEVHNAKTKPREEQYNSTYRVVSVLTVLHLDWLNGKEYKCKVSNKALPAPIEETIFKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 9 aa, synthetic construct
FNIKDTYMH

SEQ ID NO: 33 - protein, 17 aa, synthetic construct
RIDPASDDTKYDPKFQG

SEQ ID NO: 34 - protein, 6 aa, synthetic construct
DGYYAY

SEQ ID NO: 35 - protein, 15 aa, synthetic construct
RASESVDSYGNSFMH

SEQ ID NO: 36 - protein, 7 aa, synthetic construct
RASNLES

SEQ ID NO: 37 - protein, 9 aa, synthetic construct
QQSYEVPRT

SEQ ID NO: 38 - protein, 9 aa, synthetic construct
YTFTAYWMH

SEQ ID NO: 39 - protein, 17 aa, synthetic construct
AIYPGNSDAGYNQKFKG

SEQ ID NO: 40 - protein, 6 aa, synthetic construct
WGNNSF

SEQ ID NO: 41 - protein, 11 aa, synthetic construct
KASQDINSYLT

SEQ ID NO: 42 - protein, 7 aa, synthetic construct
RANRLVD

SEQ ID NO: 43 - protein, 9 aa, synthetic construct
LQYDEFPPT

SEQ ID NO: 44 - protein, 9 aa, synthetic construct
YAFTNYLIE

SEQ ID NO: 45 - protein, 17 aa, synthetic construct
VINPGSGGTNYNEKFKG

SEQ ID NO: 46 - protein, 7 aa, synthetic construct
QVGRLAY

SEQ ID NO: 47 - protein, 16 aa, synthetic construct
KSSRSLLDSDGRTYLN

SEQ ID NO: 48 - protein, 7 aa, synthetic construct
LVSKLDS

SEQ ID NO: 49 - protein, 9 aa, synthetic construct
WQGTHFPPT

SEQ ID NO: 50 - protein, 8 aa, synthetic construct
GFNIKDTY

SEQ ID NO: 51 - protein, 8 aa, synthetic construct
IDPASDDT

SEQ ID NO: 52 - protein, 8 aa, synthetic construct
SKDGYYAY

SEQ ID NO: 53 - protein, 10 aa, synthetic construct
ESVDSYGNSF

SEQ ID NO: 54 - skipped

SEQ ID NO: 55 - protein, 8 aa, synthetic construct
GYTFTAYW

SEQ ID NO: 56 - protein, 8 aa, synthetic construct
IYPGNSDA

SEQ ID NO: 57 - protein, 8 aa, synthetic construct
TRWGNNSF

SEQ ID NO: 58 - protein, 6 aa, synthetic construct
QDINSY

SEQ ID NO: 59 - skipped

SEQ ID NO: 60 - protein, 8 aa, synthetic construct
GYAFTNYL

SEQ ID NO: 61 - protein, 8 aa, synthetic construct
INPGSGGT

SEQ ID NO: 62 - protein, 9 aa, synthetic construct
VLQVGRLAY

SEQ ID NO: 63 - protein, 11 aa, synthetic construct
RSLLDSDGRTY

SEQ ID NO: 64 - skipped

SEQ ID NO: 65 - protein, 9 aa, synthetic construct
WQGTHFPPT

SEQ ID NO: 66 - protein, 115 aa, synthetic construct
QVQLQQSGTELVKPGASVKLSCTASGFNIKDTYMHWVKQRPEQGLEWIGRIDPASDDTKYDPKFQGKAAITADTSSNTAYLQLSSLTSEDTAVYYCSKDGYYAYWGQGTLVTVSA

SEQ ID NO: 67 - protein, 112 aa, synthetic construct
DIVMTQSPASLAVSLGQRATISCRASESVDSYGNSFMHWYQQKPGQPPKLLIYRASNLESGIPARFSGSGSRTDFTLTINPVEADDVATYYCQQSYEVPRTFGGGTKLEIKR

SEQ ID NO: 68 - protein, 115 aa, synthetic construct
QVQLQQSGTVLARPGASVKMSCKASGYTFTAYWMHWVKQRPGQGLEWIGAIYPGNSDAGYNQKFKGKAKLTAVTSTSTAYMELSSLANEDSAVYYCTRWGNNSFWGQGTTLTVSS

SEQ ID NO: 69 - protein, 108 aa, synthetic construct
DIQMTQSPSSMYASLGERVTITCKASQDINSYLTWFQQKPGKSPKTLIYRANRLVDGVPSRFSGSGSGQDYSLTISTLEYEDMGIYYCLQYDEFPPTFGAGTKLELKR

SEQ ID NO: 70 - protein, 116 aa, synthetic construct
QVQLQQSGAELVRPGTSVKVSCKTSGYAFTNYLIEWVKQRPGQGLEWIGVINPGSGGTNYNEKFKGKATLTADKSSSTAYMQLSSLTSDDSAVYFCVLQVGRLAYWGQGTLVTVSA

SEQ ID NO: 71 - protein, 113 aa, synthetic construct
DIQMTQSPLTLSVTIGQPASISCKSSRSLLDSDGRTYLNWLLQRPGQSPKRLIYLVSKLDSGVPDRFTGSGSGTDFTLKISRVEAEDLGVYYCWQGTHFPPTFGAGTKLELKR

(19) United States
(12) Patent Application Publication
(10) Pub. No.: US 2025/0127885 A1
(43) Pub. Date: Apr. 24, 2025

(54) ENGINEERED FC DOMAINS AND ENGINEERED HCMV VIRAL FC RECEPTORS

(71) Applicant: BOARD OF REGENTS, THE UNIVERSITY OF TEXAS SYSTEM, Austin, TX (US)

(72) Inventors: Jennifer MAYNARD, Austin, TX (US); Ahlam QERQEZ, Cedar Park, TX (US); Alison G. LEE, Austin, TX (US); Sumit PAREEK, Austin, TX (US)

(73) Assignee: BOARD OF REGENTS, THE UNIVERSITY OF TEXAS SYSTEM, Austin, TX (US)

(21) Appl. No.: 18/718,249

(22) PCT Filed: Dec. 9, 2022
(86) PCT No.: PCT/US2022/081258

§ 371 Date: Jun. 10, 2024

Related U.S. Application Data
(60) Provisional application No. 63/288,254, filed on Dec. 10, 2021.

(51) Int. Cl. A61K 39/245; C07K 14/005

(52) U.S. Cl. CPC A61K 39/245; C07K 14/005; C07K 2319/30; C12N 2710/16122; C12N 2710/16134

(57) ABSTRACT

Recombinant antibodies are provided that comprise engineered human IgG1 Fc domains that have reduced affinity for viral FcγRs, such as HCMV gp34 and gp68, as compared to a wild-type human IgG1 Fc domain. The engineered human IgG1 Fc domains have equivalent affinity for CD16A and FcRn as compared to a wild-type human IgG1 Fc domain. Also provided are engineered forms of HCMV gp34 and their use in a vaccine.

### REFERENCE TO RELATED APPLICATIONS

The present application claims the priority benefit of U.S. provisional application No. 63/288,254, filed Dec. 10, 2021, the entire contents of which are incorporated herein by reference.

### REFERENCE TO A SEQUENCE LISTING

This application contains a Sequence Listing XML, which has been submitted electronically and is hereby incorporated by reference in its entirety. Said Sequence Listing XML, created on Nov. 18, 2022, is named UTFBP1300WO_ST26.xml and is 71,931 bytes in size.

### BACKGROUND

### 1. Field

The present invention relates generally to the fields of medicine, immunology, and virology. More particularly, it concerns engineered human IgG1 Fc domains having reduced affinity for viral FcγRs, such as gp34 and gp68 of human cytomegalovirus, as well as gpRL12 and gpRL13 of human cytomegalovirus and the functionally homologous gE/gI of herpes simplex 1 (HSV-1), herpes simplex 2 (HSV-2), and varicella zoster (VZV).

### 2. Description of Related Art

Infection by human cytomegalovirus (HCMV) is widespread and poses risks for the very young, old, and immuno-compromised. In 1999, the U.S. Institute of Medicine ranked development of an HCMV vaccine as the top vaccine priority based on treatment costs (Plotkin, 2015, Modlin et al., 2004). Despite ongoing efforts in vaccine development for >50 years, the leading glycoprotein B (gB) vaccine reported only 43-50% efficacy in HCMV-negative adolescent girls (Bernstein et al., 2016), sero-negative women (Pass et al., 2009), and solid organ transplant recipients (Griffiths et al., 2011). Unexpectedly, protection correlated with the presence of high levels of non-neutralizing antibodies in these studies (Nelson et al., 2018).

Antibody Fc-mediated functions, such as antibody-dependent cellular cytotoxicity (ADCC) by natural killer (NK) cells and antibody-dependent phagocytosis (ADCP) by neutrophils and myeloid cells, contribute to protection against a variety of viruses, including HCMV (Nelson et al., 2018, Horwitz et al., 2017, DiLillo et al., 2016). HCMV's primarily cell-associated properties suggest that in addition to T cells, NK cells may contribute to clearance of infected cells. Dramatic expansions of NKG2C⁺/CD57⁺ and FcεR1γ-negative adaptive NK cells, which efficiently mediate ADCC, occur after HCMV infection (Aicheler et al., 2013, Costa-Garcia et al., 2015). NK cells can prevent HCMV infection, super-infection of seropositive individuals with a second strain (Britt, 2017), and viral spread of cultured cells in vitro (Wu et al., 2015a). Conversely, individuals with impaired NK immunity are susceptible to infection (Aicheler et al., 2013). Overall, NK cells appear to be necessary for both innate and adaptive immune responses against the spread of HCMV.

Despite the potential for these strategies to control infection, HCMV is adept at evading the immune system, leading to alternating phases of active and latent infection of most adults worldwide. The HCMV genome includes multiple genes that support NK cell evasion by expressing inhibitory receptors (Forrest et al., 2020) and suppressing interferon release (Patel et al., 2018). Considerable clinical evidence indicates that anti-HCMV antibodies poorly prevent viral spread. Clinical strains spread primarily from cell-to-cell which is not inhibited in vitro by seropositive sera despite robust antibody responses (Falk et al., 2018). In addition to super-infection, approximately 1% of seropositive mothers transmit HCMV to their fetuses (Britt, 2017) and it is unclear whether administration of HCMV intravenous immunoglobulin to seronegative pregnant women prevents vertical transmission (Nigro et al., 2005, Revello et al., 2014).

Potent anti-HCMV therapeutics and vaccines are needed to prevent viral spread. For example, composition and methods to enhance neutralization of cell-to-cell spread and NK cell clearance of infected cells are needed.

### SUMMARY

A recombinant antibody comprising (a) a variable domain that selectively binds to an immunogenic viral antigen; and (b) an engineered human Fc domain having reduced affinity for one or more viral Fc receptor(s) as compared to a wild-type human Fc domain. In some aspects, the engineered human Fc domain is an engineered human IgG1 Fc domain.

The viral antigen may be an antigen from a virus in the Herpesviridae family, such as, for example, an HCMV antigen, an HSV-1 antigen, an HSV-2 antigen, or a varicella zoster antigen. The viral Fc receptor may be a herpes viral Fc receptor, such as, for example, gp34, gp68 gpRL12, gpRL13, and/or gE/gI.

In some aspects, the engineered human Fc domain (e.g., IgG1 Fc domain) has similar affinity for CD16A (V158 and F158) and FcRn as compared to a wild-type human IgG1 Fc domain. In some aspects, the engineered human Fc domain (e.g., IgG1 Fc domain) has an affinity for CD16A and/or FcRn that is no more than 2-fold, no more than 1.9-fold, no more than 1.8-fold, no more than 1.7-fold, no more than 1.6-fold, no more than 1.5-fold, no more than 1.4-fold, no more than 1.3-fold, or no more than 1.2-fold different from the affinity of a wild-type IgG1 Fc domain for CD16A and/or FcRn.

In some aspects, the engineered human Fc domain (e.g., IgG1 Fc domain) has at least about 5-fold, at least about 6-fold, at least about 7-fold, at least about 8-fold, at least about 9-fold, at least about 10-fold, at least about 15-fold, at least about 20-fold lower, at least about 25-fold lower, at least about 30-fold lower, at least about 35-fold lower, at least about 40-fold lower, at least about 45-fold lower, or at least about 50-fold lower affinity for gp34 as compared to a wild-type human IgG1 Fc domain.

In some aspects, the engineered human Fc domain (e.g., IgG1 Fc domain) has at least about 5-fold, at least about 6-fold, at least about 7-fold, at least about 8-fold, at least about 9-fold, at least about 10-fold, at least about 15-fold, at least about 20-fold lower, at least about 25-fold lower, at least about 30-fold lower, at least about 35-fold lower, at least about 40-fold lower, at least about 45-fold lower, or at least about 50-fold lower affinity for gp68 as compared to a wild-type human IgG1 Fc domain.

In some aspects, the engineered human Fc domain (e.g., IgG1 Fc domain) has at least about 5-fold, at least about 6-fold, at least about 7-fold, at least about 8-fold, at least about 9-fold, at least about 10-fold, at least about 15-fold, at least about 20-fold lower, at least about 25-fold lower, at least about 30-fold lower, at least about 35-fold lower, at least about 40-fold lower, at least about 45-fold lower, or at least about 50-fold lower affinity for gpRL12 as compared to a wild-type human IgG1 Fc domain.

In some aspects, the engineered human Fc domain (e.g., IgG1 Fc domain) has at least about 5-fold, at least about 6-fold, at least about 7-fold, at least about 8-fold, at least about 9-fold, at least about 10-fold, at least about 15-fold, at least about 20-fold lower, at least about 25-fold lower, at least about 30-fold lower, at least about 35-fold lower, at least about 40-fold lower, at least about 45-fold lower, or at least about 50-fold lower affinity for gpRL13 as compared to a wild-type human IgG1 Fc domain.

In some aspects, the engineered human Fc domain (e.g., IgG1 Fc domain) comprises a substitution at the following position(s): R255, H268, E294, Q311, K334, and/or S337, wherein the positions are numbered according to the EU numbering system for human IgG1 (which correspond to positions 28, 41, 67, 84, 107, and 110, respectively, of SEQ ID NOs: 1-5).

In some aspects, the engineered human Fc domain (e.g., IgG1 Fc domain) comprises a substitution at the following position(s): R255, H268, Q311, and/or K334, wherein the positions are numbered according to the EU numbering system for human IgG1. In some aspects, the engineered human Fc domain (e.g., IgG1 Fc domain) comprises the following substitution(s): R255Q, H268L, Q311L, and/or K334E, wherein the positions are numbered according to the EU numbering system for human IgG1. In some aspects, the engineered human IgG1 Fc domain comprises an amino acid sequence of SEQ ID NO: 28 or 29. In some aspects, the engineered human Fc domain (e.g., IgG1 Fc domain) has at least about 50-fold lower affinity for both gp34 and gp68 as compared to a wild-type human IgG1 Fc domain.

In some aspects, the engineered human Fc domain (e.g., IgG1 Fc domain) comprises a substitution at the following position(s): R255, H268, Q311, K334, and/or S337, wherein the positions are numbered according to the EU numbering system for human IgG1. In some aspects, the engineered human Fc domain (e.g., IgG1 Fc domain) comprises the following substitution(s): R255Q, H268L, Q311L, K334E, and/or S337F, wherein the positions are numbered according to the EU numbering system for human IgG1. In some aspects, the engineered human IgG1 Fc domain comprises an amino acid sequence of SEQ ID NO: 30 or 31. In some aspects, the engineered human Fc domain (e.g., IgG1 Fc domain) has at least about 50-fold lower affinity for both gp34 and gp68 as compared to a wild-type human IgG1 Fc domain. In some aspects, the engineered human Fc domain (e.g., IgG1 Fc domain) has improved thermal stability as compared to the engineered human IgG1 Fc domain comprises an amino acid sequence of SEQ ID NO: 4 or 6. In some aspects, the engineered human Fc domain (e.g., IgG1 Fc domain) has similar clearance kinetics as a wild-type human IgG1 Fc domain.

In some aspects, the engineered human Fc domain (e.g., IgG1 Fc domain) comprises a substitution at the following position(s): R255, H268, E294, Q311, and/or K334, wherein the positions are numbered according to the EU numbering system for human IgG1. In some aspects, the engineered human Fc domain (e.g., IgG1 Fc domain) comprises the following substitution(s): R255Q, H268L, E294K, Q311L, and/or K334E, wherein the positions are numbered according to the EU numbering system for human IgG1. In some aspects, the engineered human IgG1 Fc domain comprises an amino acid sequence of SEQ ID NO: 4 or 6. In some aspects, the engineered human Fc domain (e.g., IgG1 Fc domain) has at least about 50-fold lower affinity for both gp34 and gp68 as compared to a wild-type human IgG1 Fc domain. In some aspects, the engineered human Fc domain (e.g., IgG1 Fc domain) has lower affinity for gpRL12 as compared to a wild-type human IgG1 Fc domain.

In some aspects, the engineered human Fc domain (e.g., IgG1 Fc domain) comprises a substitution at the following position(s): R255 and/or S337, wherein the positions are numbered according to the EU numbering system for human IgG1. In some aspects, the engineered human Fc domain (e.g., IgG1 Fc domain) comprises the following substitution(s): R255Q and/or S337F, wherein the positions are numbered according to the EU numbering system for human IgG1. In some aspects, the engineered human IgG1 Fc domain comprises an amino acid sequence of SEQ ID NO: 5 or 7. In some aspects, the engineered human Fc domain (e.g., IgG1 Fc domain) has at least about 5-fold lower affinity for gp34 as compared to a wild-type human IgG1 Fc domain. In some aspects, the engineered human Fc domain (e.g., IgG1 Fc domain) has at least about 20-fold lower affinity for gp68 as compared to a wild-type human IgG1 Fc domain. In some aspects, the engineered human Fc domain (e.g., IgG1 Fc domain) has lower affinity for gpRL13 as compared to a wild-type human IgG1 Fc domain.

In some aspects, the engineered human Fc domain (e.g., IgG1 Fc domain) further comprises at least one substitution to increase Fc effector functions. In some aspects, the Fc effector function is antibody-dependent cellular cytotoxicity (ADCC) or antibody-dependent phagocytosis (ADCP). In some aspects, the at least one substitution increases binding to classic host Fc receptors, such as, for example, CD16a and/or CD32a. In some aspects, the substitution is one or more of G236A, S239D, A330L, and/or 1332E, wherein the positions are numbered according to the EU numbering system for human IgG1.

In some aspects, the engineered human Fc domain (e.g., IgG1 Fc domain) further comprises at least one substitution that alters affinity for FcRn. In some aspects, the engineered human Fc domain (e.g., IgG1 Fc domain) further comprises the following substitution(s): M252Y, S254T, and/or T256E, wherein the positions are numbered according to the EU numbering system for human IgG1. In some aspects, the engineered human Fc domain (e.g., IgG1 Fc domain) further comprises the following substitution(s): L309D, Q311H, and/or N434S, wherein the positions are numbered according to the EU numbering system for human IgG1. In some aspects, the engineered human Fc domain (e.g., IgG1 Fc domain) further comprises the following substitution(s): M428L and/or N434S, wherein the positions are numbered according to the EU numbering system for human IgG1.

In some aspects, the recombinant antibody is glycosylated.

In some aspects, the immunogenic viral antigen is an immunogenic HCMV, HSV-1, HSV-2, or varicella zoster (VZV) antigen. The immunogenic HCMV antigen may be an HCMV glycoprotein. In some aspects, the immunogenic HMCV antigen is an gH, gL, gB, gO, gN, gM, UL83, UL123, UL128, UL130 and UL131A, or pp65 antigen.

In some aspects, the recombinant antibody selectively interacts with the immunogenic HMCV antigen as expressed on HMCV-infected cells. In some aspects, the immunogenic HMCV antigen is gp34, HCMV antigen gp68, HCMV antigen RL12, HCMV antigen RL13, HSV-1 antigen gE/gI, HSV-2 antigen gE/gI, or VZV antigen gE/gI. In some aspects, the variable domain comprises clone-paired heavy and light chain CDR sequences derived from the clone-paired heavy chain and light chain variable sequences of Table 6. In some aspects, the variable domain comprises clone-paired heavy and light chain CDR sequences from Tables 4 or 5. In some aspects, the antibody or antibody fragment comprises clone-paired heavy chain and light chain variable sequences having, independently, at least 70%, 80%, or 90% identity to sequences from Table 6. In some aspects, the antibody or antibody fragment comprises clone-paired heavy chain and light chain variable sequences each having at least 95% identity to sequences from Table 6. In some aspects, the antibody or antibody fragment comprises clone-paired heavy chain and light chain variable sequences from Table 6.

In some aspects, the recombinant antibody is a chimeric antibody, bispecific antibody, or BiTE. In some aspects, the recombinant antibody is a human antibody or humanized antibody. In some aspects, the recombinant antibody is an IgG1, IgG2, IgG3, IgG4, IgM, or IgA antibody.

In some aspects, the recombinant antibody is fused to an imaging agent. In some aspects, the recombinant antibody is labeled. In some aspects, the label is a fluorescent label, an enzymatic label, or a radioactive label. In some aspects, the recombinant antibody is coupled to a therapeutic, a reporter, or a targeting moiety.

In one embodiment, provided herein are isolated nucleic acids encoding a recombinant antibody provided herein. In some aspects, the isolated nucleic acids are DNA. In some aspects, the nucleic acids are one or more mRNA.

In one embodiment, provided herein are expression vectors comprising nucleic acids encoding a recombinant antibody provided herein.

In one embodiment, provided herein are hybridomas or engineered cells comprising nucleic acids encoding a recombinant antibody provided herein.

In one embodiment, provided herein are methods of making recombinant antibody provided herein, the method comprising culturing a hybridoma or engineered cell comprising nucleic acids encoding the recombinant antibody under conditions that allow expression of the recombinant antibody and optionally isolating the recombinant antibody from the culture.

In one embodiment, provided herein are pharmaceutical formulations comprising one or more recombinant antibody provided herein or one or more mRNA encoded a recombinant antibody provided herein.

In one embodiment, provided herein are methods of treating a subject comprising administering an effective amount of a pharmaceutical formulation provided herein to the subject. In some aspects, the subject has an HCMV infection. In some aspects, the subject is at risk for an HCMV infection. In some aspects, the subject is a transplant patient. In some aspects, the subject is an elderly patient. In some aspects, the subject is a CMV-seronegative pregnant woman.

In some aspects, the methods provide for selective targeting of HCMV-infected cells as compared to targeting of healthy cells. In some aspects, the methods induce NK cell and macrophage activation against HCMV-infected cells. In some aspects, the methods prevent cell-to-cell spread of HCMV within the subject. In some aspects, the methods induce antibody dependent cellular cytotoxicity (ADCC), complement-dependent cellular cytoxicity and antibody-dependent cellular trogocytosis against HCMV-infected cells and complement lysis and antibody-dependent cellular phagocytosis (ADCP) against virions.

In one embodiment, provided herein are engineered proteins comprising an engineered HCMV gp34 protein ectodomain that comprises a C150S substitution, with the position being relative to SEQ ID NO: 11. In some aspects, the HCMV gp34 protein ectodomain comprises a sequence at least 95% identical to amino acids 24-182 of SEQ ID NO: 11. In some aspects, the engineered protein is soluble. In some aspects, the engineered protein comprises a sequence at least 95% identical to SEQ ID NO: 16.

In one embodiment, provided herein are nucleic acid molecules comprising a nucleotide sequence that encodes an amino acid sequence of an engineered protein provided herein. In some aspects, the nucleic acid molecule is a DNA molecule. In some aspects, the nucleic acid molecule is an RNA molecule. In some aspects, the nucleic acid molecule is an mRNA molecule.

In one embodiment, provided herein are expression vectors comprising nucleic acids encoding an amino acid sequence of an engineered protein provided herein.

In one embodiment, provided herein are engineered cells comprising a nucleic acid encoding an engineered protein provided herein. In some aspects, the cell is a CHO cell.

In one embodiment, provided herein are methods of making an engineered protein provided herein, the method comprising culturing an engineered cell comprising a nucleic acid encoding an engineered protein provided herein under conditions that allow expression of the engineered protein and optionally isolating the engineered protein from the culture.

In one embodiment, provided herein are pharmaceutical compositions comprising a pharmaceutically acceptable carrier; and (i) an engineered protein provided herein, or (ii) an mRNA encoding the engineered protein. In some aspects, the compositions further comprise an adjuvant.

In one embodiment, provided herein are methods of preventing HCMV infection or a disease associate with HCMV infection in a subject, comprising administering to the subject an effective amount of the pharmaceutical composition comprising a pharmaceutically acceptable carrier; and (i) an engineered protein provided herein, or (ii) an mRNA encoding the engineered protein.

In one embodiment, provided herein are compositions comprising an engineered protein provided herein, bound to an antibody.

In one embodiment, provided herein are monoclonal antibodies that bind to an engineered HCMV gp34 protein provided herein. In some aspects, the antibody or antibody fragment comprises clone-paired heavy and light chain CDR sequences derived from the clone-paired heavy chain and light chain variable sequences of Table 6. In some aspects, the variable domain comprises clone-paired heavy and light chain CDR sequences from Tables 4 or 5. In some aspects, the antibody or antibody fragment comprises clone-paired heavy chain and light chain variable sequences having, independently, at least 70%, 80%, or 90% identity to sequences from Table 6. In some aspects, the antibody or antibody fragment comprises clone-paired heavy chain and light chain variable sequences each having at least 95% identity to sequences from Table 6. In some aspects, the antibody or antibody fragment comprises clone-paired heavy chain and light chain variable sequences from Table 6.

In some aspects, the antibody fragment is a recombinant scFv (single chain fragment variable) antibody, Fab fragment, F(ab′)₂ fragment, or Fv fragment. In some aspects, the antibody is a chimeric antibody or a bispecific antibody. In some aspects, the antibody is a humanized antibody. In some aspects, the antibody is capable of binding to HCMV gp34 protein. In some aspects, the antibody is an IgG antibody or a recombinant IgG antibody or antibody fragment. In some aspects, the antibody is an IgG1, IgG2, IgG3, IgG4, IgM, or IgA antibody. In some aspects, the antibody comprises an engineered Fc domain as provided herein. In some aspects, the antibody or antibody fragment is fused to an imaging agent. In some aspects, the antibody or antibody fragment is labeled. In some aspects, the label is a fluorescent label, an enzymatic label, or a radioactive label. Also provided are isolated nucleic acids and expression vectors encoding the antibody, hybridomas expressing the antibody, and methods of making the antibody.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-D: Characterization of anti-gB CD16A activation and internalization with HCMV infected cells. (FIG. 1A) BW-CD16A-ζ were incubated with MRC5 fibroblasts infected (MOI=5, 72 hpi) with HCMV strains, AD169 or vFcγR-deleted (ΔΔΔ), in the presence of human monoclonal SM5-1 (anti-gB), Cytotec (polyclonal anti-HCMV, 1/100 dilution), or isotype control (rituximab. 100 μg/mL). CD16A activation was measured by m-IL2 release from reporter cells and measured using ELISA. Data are mean±SD, n=3. (FIG. 1B) Fluorescent images of AD169 infected fibroblasts (MOI=2, 96 hpi) incubated with 20 μg/mL AF647-labelled SM5-1 hu-Fc or mouse-Fc for 2 hours at 37° C. Cells were fixed with 4% PFA and scanned using Zeiss LSM 710/Elyra S.1 at 63X. (FIG. 1C-D) Antibody binding (C) and internalization (D) by incubating infected fibroblasts (AD169 or ΔΔΔ, MOI=2, 96 hpi) or uninfected (mock) with 67 nM pHrodo-Red-labelled hu-Fc (SM51 or isotype control) for 2 hours at 37° C. Extracellular antibody was detected with goat anti-human-Fcγ AF647. Data are mean±SD, n=2. *p<0.05, **p<0.01, ***p<0.001, ****p<0.0001, ns: non-significant. Two-way ANOVA followed by Tukey's multiple comparisons test in GraphPad. Representative data of one experiment is shown; each experiment was repeated twice.

FIGS. 2A-K: Characterization of soluble gp68 and gp34 ectodomains. (FIG. 2A) Competition ELISA in which hu-Fc binding was measured by detecting FLAG-tagged ligand (CD16A-GST, FcRn-GST, or gp34-M) in the presence of serially diluted unlabeled competing ligands (gp34-M, t-gp68, hu-Fc). Area under the curve (AUC) normalized to no competition was plotted. (FIG. 2B) Size exclusion chromatography (S200) of t-gp68, gp34-M, Fc, and complex incubated at 3:1:1 molar ratio, respectively. Molecular weight standards indicated by circles (blue dextran, thyroglobulin, ferritin, beta amylase, aldolase, conalbumin, ovalbumin, carbonic anhydrase, cytochrome C). (FIG. 2C) SDS-PAGE (4-20%) of complex from FIG. 2A, in reduced (R) conditions. 3D reconstruction and 2D class averages for particles containing: (FIG. 2D) Two Fcs flanking a gp34-M dimer with interactions at the CH₂ apex. The appendages in the 2D images correspond to t-gp68 bound to CH₂—CH₃ interface with partial occupancy. (FIG. 2E) One Fc bound to gp34-M at the CH₂ tip. (FIG. 2F) One Fc bound to gp68 at the CH₂—CH₃ interface. A RoseTTAFold based gp34 and gp68 model was used to fit the 3D reconstruction. Fc crystal structure (2GJ7) was used. Surface plasmon resonance to measure FIG. 2G. Fc/gp68 binding kinetics with hu-Fc coupled to a CM5 chip at 200 RU. Different concentrations of t-gp68 were injected ranging from 250 to 3 nM in 2-fold dilutions. (FIG. 2H) Fc/gp34-M binding kinetics, gp34 C150S was injected at 40 RU to an anti-strep Fab coated chip followed by injections of hu4D5 from 200 to 3.125 nM in 2-fold dilutions. K_{D}s were calculated using 1:1 model in the BIAevaluation X100 software for both receptors. (FIGS. 2I-K) The ability of soluble vFcγR ectodomains (+/−2 μM of t-gp68 and/or 0.1 M of gp34-M) to inhibit 67 nM pHrodo-Red-labelled SM5-1 or isotype hu-Fc binding to (FIG. 2J) and internalization (FIG. 2I) by AD169 infected MRC5s (MOI=2, 96 hpi). In both graphs, the bars in each Fab group represent, from left to right, human IgG1, +gp34-M, +t-gp68, gp34-M+t-gp68, and mouse IgG2A. A diagram of the assay set-up is provided in FIG. 2K. Extracellular antibody detected with goat anti-human-Fcγ AF647. The grey dashed line represents threshold for internalization. Data are mean±SD, n=2. *p<0.05, **p<0.01, ***p<0.001, ****p<0.0001, ns: non-significant. All analysis was done using GraphPad. Two-way ANOVA followed by Tukey's multiple comparisons test. Representative data of one experiment shown; each experiment was repeated at least twice.

FIG. 3A-F. Engineering Fc domains for loss of vFcγR binding. (FIG. 3A) Yeast display and staining strategy of hu-Fc. (FIG. 3B) An error prone library (1%) on CH₂ of Fc was displayed on yeast. FACs sorting was performed in the presence of soluble tetramerized AF647 CD16A (V158) in presence of unlabeled 1 μM gp34-M. (FIG. 3C) Another error prone (1%) library was generated using R47 mutant as the template sequence. Sorting was performed with soluble tetramerized AF647-CD16A (V158) and PE-FcRn at pH 6.0 in presence of unlabeled 5 μM t-gp68. (FIG. 3D) K_{D}s for host and vFcγR were determined by BLI (FcRn, CD16AV158 or F158) and SPR (gp34-M, t-gp68) with hu4D5 Fc variants. The mean±SEM (n=2) K_{D} values are shown. Each group of three values represent, from left to right, WT, G2, and G5. (FIG. 3E) vFcγR ectodomains were expressed on ExpiCHO cells and stained with hu4D5 Fc variants (WT, G2, and G5) in a dose dependent manner (n=2), with binding detected using goat-anti-Fcγ-AF647. Mean area under the curve (AUC), n=2, was calculated for all curves. Experiment was repeated twice. For FIGS. 3D-E (compared to WT) one-way ANOVA with Tukey test multiple comparisons was used, **p<0.01, ***p<0.001, ****p<0.0001, ns: non-significant. (FIG. 3F) Size exclusion chromatography (S200) of SM5-1 Fc variants. The right-shifted curve is G5.

FIGS. 4A-E. Selected Fc variants retain host FcγR-driven biological activities. (FIG. 4A) ADCC assay was measured by calcein release of SKOV3 cells incubated with NK-92 F158 or V158 at 10:1 (E:T) ratio with hu4D5 Fc variants (WT, G2, G5). (FIG. 4B-C) Phagocytosis by THP-1 monocytes was performed using fluorescent gB coated beads (50:1 beads per cell) (FIG. 4B) or with fluorescent AD169-virions (2 virions per cell) with SM5-1 Fc variants (FIG. 4C). All ADCP data are mean±SD (n=2) and statistical analysis was performed using two-way ANOVA followed by Tukey's multiple comparison test, *p<0.05, **p<0.01, ***p<0.001, ****p<0.0001, ns: non-significant in GraphPad. Representative data of one experiment is shown and each experiment was repeated at least twice. (FIG. 4D) Serum antibody concentration in homozygous Tg32 transgenic mice (n=3-4) as a function of time after administration. Each hu4D5 Fc variant (WT, G2, G5, IHH) was administered intraperitoneally at 2 mg/kg, with sera antibody concentrations determined by antigen specific ELISA. Data are presented as mean±SD (n=3-4). (FIG. 4E) Thermal stability was measured using differential scanning fluorimetry. The lines represent, from top to bottom at 60° C., G2, G5, and WT.

FIGS. 5A-F. Anti-gB antibodies with vFcγR-resistant Fcs show enhanced anti-viral activities. (FIGS. 5A-B) Cell staining (A) and internalization (B) of SM5-1 Fc variants in the presence of AD169- or mock-infected MRC5. One-way ANOVA with Tukey's multiple comparisons was used for statistical analysis. Data are presented as mean±SD (n=2). For each pair of columns, the left is AD169 and the right is mock. (FIG. 5C) CD16A activation of SM5-1 Fc variants measured by mIL-2 secretion of BW-CD16A-ζ cells in the presence of AD169- or ΔΔΔ infected MRC5 (MOI=5, 72 hpi). Curves were fit to 4PL or AUC using GraphPad. In each line graph, the top line is ΔΔΔ and the bottom line is AD169. In the bar graph, the left bar in each pair is AD169 and the right bar in each pair is ΔΔΔ. Mean EC₅₀ for each antibody is shown and ND indicates no activation at 100 μg/mL. p values indicate differences in EC₅₀ for activation in the presence of AD169 and ΔΔΔ. Asterisks next to EC₅₀ represent comparisons to SM5-1 WT. The AUC was normalized to SM5-1 WT activation with ΔΔΔ. Data is shown as the mean±SEM of 3 experiments. (FIGS. 5D-E) Percentage of NK-92 (V158 (D) or F158 (E)) degranulation in the presence of AD169 infected MRC5 (MOI=2, 96 hpi) with SM5-1 Fc variants. Data are presented as mean±SD (n=2), two-way ANOVA with Tukey's multiple comparisons test. *p<0.05, **p<0.01, ***p<0.001, ****p<0.0001, ns=non-significant. (FIG. 5F) Correlation generated using GraphPad with EC50 of CD16A activation with SM5-1 Fc-variants in the presence of AD169 infected cells (see FIG. 16A) and AUC of vFcγR-Fc binding ELISAS (see FIG. 11D). In the gp34 graph, reading from left to right along the X-axis, the data points represent G2, R47, G5, S337F, WT, and R255Q. In the gp68 graph, reading from left to right along the X-axis, the data points represent G2, G5, R255Q, S337F, R47, and WT. Pearson's coefficient and linear regression fit are shown.

FIGS. 6A-D. SM5-1 is used to measure gB expression and CD16A activation in wild-type and vFcγR-deleted AD169 strains. AD169 wild-type or vFcγR-deleted, ΔΔΔ, were used to infect MRC5 (MOI=5, 72 hpi). Uninfected MRC5 cells were used as a control. (FIG. 6A) Cells were stained with directly labeled 10 nM anti-gB SM5-1 mFc (AF647), 1/50 anti-HLA-A2 (APC), or 1/50 anti-HLA-A/B/C (APC) on ice. Other samples were stained with 1/50 Cytotec followed by secondary staining with goat-anti-human Fc (AF647). Samples were scanned on BD Fortessa and normalized histograms were plotted. gB expression was unaltered in AD169 and isogenic ΔΔΔ virus. HLA-A2 was downregulated in the presence of infected cells compared to uninfected, as predicted. HLA-A/B/C were similarly expressed on infected and uninfected cells. Positive cells are gated and percentages are listed above. (FIG. 6B) Western blot was performed on cell lysate from infected cells. In all samples except for uninfected, gB expression was present (SM5-1 mFc). IE1/IE2 expression was observed for all infected cells and β actin was used as a control. (FIGS. 6C-D) Time course of CD16A activation in the presence infection at different time points (24, 48, 72 hpi) in the presence of Cytotec and rituximab (control) at 100 μg/mL (C), or SM5-1 (D). Data are presented as mean±SD (n=2) repeated twice. Statistical analysis was performed by GraphPad using two-way ANOVA followed by Tukey's multiple comparison test, *p<0.05, **p<0.01, ***p<0.001, ****p<0.0001, ns: non-significant.

FIGS. 7A-F. Biochemical characterization of t-gp68, wild-type (WT) gp34, and gp34-M. (FIG. 7A) SEC (5200) chromatogram and SDS-PAGE (4-20%, reducing or non-reducing conditions) for t-gp68. (FIG. 7B) The native gp34 sequence includes multiple N-linked glycosylation sites (Y) and cysteines (0). Modifications in the recombinant truncated gp34 and gp34-M variants shown, including the inferred di-sulfide bonding pattern. (FIG. 7C) SEC chromatograms for gp34 and gp34-M on an S200 column, 100 pg injections. (FIG. 7D) SDS-PAGE (4-20%) of WT gp34 and gp34-M under reduced (R) or non-reduced (NR) conditions, 3 μg per lane. The right-shifted line represents gp34-M. (FIG. 7E) Static light scattering (SLS) of purified Fc and gp34-M was used to estimate the approximate molecular weight from the reciprocal y-intercept. Fc as 51±0.3 kDa and gp34-M as 84±1 kDa. The bottom line represents gp34-M. (FIG. 7F) ELISA binding of plate coated human Fc to purified FLAG tagged WT gp34 and gp34-M detected using anti-FLAG (M2)-HRP. Taken at 10 μg/mL, the top line is WT gp34. Representative data of one experiment is shown and each experiment was repeated at least twice with technical replicates.

FIGS. 8A-D. Characterizing Fc binding to viral Fc receptors, gp34 and gp68. (FIGS. 8A-B) ELISA was used to determine whether known Fc variants with altered FcRn binding impact vFcγR binding. No coat or coated receptors (t-gp68 (A&B), gp34-M (A&B), FcRn-GST (A), CD16-GST (B)) were used in the presence of hu4D5 FcRn-altered antibodies (WT, LS, N434S, N434Y, and YTE) at multiple concentrations (333 to 0.004 μg/mL; 5-fold dilutions) (A) or hu4D5 CD16A-altered antibodies (WT, LALAPG or TM) at multiple concentrations (10 to 0.0001 μg/mL; 5-fold dilutions) (B) and binding was detected using goat-anti-kappa-HRP. For all results, 4PL fits were determined using GraphPad. Representative data are shown with all experiments were performed at least twice. (FIG. 8C) (Top) An Fc:gp34-M only complex was used to generate negative stain 2D classes showing two Fc molecules bound together by a gp34-M dimer at the CH₂ tip. (Bottom) The 3D reconstruction of the 2D class images shows the extra density in between the two Fc molecules corresponds to a gp34-M dimer (black arrows). (FIG. 8D) An Fc+gp34-M+t-gp68 complex was used to generate negative stain 2D classes showing two Fc molecules bound together by a gp34-M dimer and t-gp68 appendages with partial occupancy (boxes containing arrows) protruding from the CH₂—CH₃ interface. Arrows indicate partially occupied t-gp68 sites, with up to three t-gp68 molecules observed per complex.

FIGS. 9A-D. Biochemical characterization of vFcγR-resistant Fc variants. (FIG. 9A) CH₂ domain sequencing of selected variants from both libraries. Variants with reduced binding to gp34 (R47 and S337F), were combined with variant exhibiting reduced binding to t-gp68 (R255Q) to generate two final clones, G2 and G5. Mutations overlayed with binding epitopes of FcRn and CD16A. The sequence of gp34gp68 WT is SEQ ID NO: 27. (FIGS. 9B-C) After expression with anti-gB SM5-1 Fab arms, purified antibodies were analyzed by 4-20% SDS-PAGE under reducing (R) and non-reducing (NR) conditions (B) and monodispersity was assessed by SEC using an S200 column with 100 μg injected (C). (FIG. 9D) Purified antibodies were coated at 4 μg/ml with FLAG-tagged receptors (FcRn-GST, CD16A-GST, gp34-M or t-gp68) serially diluted over the full dose-response curve and detected by anti-FLAG-M2-HRP antibody. Area under the curve (AUC) was calculated using GraphPad, normalized to maximum AUC from data set, and plotted as a heat map. Technical replicates are indicated by error bars and experiment was performed twice, representative data from one experiment are shown.

FIGS. 10A-B. Measurement of Fc binding kinetics for soluble viral Fc receptors. SPR was performed on different hu4D5 Fc variants for twin-strep tagged t-gp68 (A) and gp34-M (B). CM5 chip was coupled with an anti-strep Fab at 4500 RU and tgp68 (twin strep) or gp34 C150S (single strep) which were injected at a final RU of 35-40. Different concentrations of hu4D5 Fc variants were injected (shown below plots) and allowed to associate for 180 seconds and dissociate for 300 seconds. 2:1 binding fits were done using the Biacore X100 Evaluation software for t-gp68 (A) and 1:1 binding fits were performed for gp34-M (B). Steady state kinetics using Biacore X100 Evaluation software were determined for t-gp68 variants; for WT huIgG1 only the top 5 (1000-62.5 nM) curves were fit, whereas for the variants all curves (4000-62.5 nM) were fit. Data set is a single representative of an experiment performed twice.

FIGS. 11A-C. Comparison of all four vFcγR expressed by Merlin CMV. (FIG. 11A) Clustal Omega protein sequence alignment of the predicted Ig-fold domains of RL11 (SEQ ID NO: 11), 12 (SEQ ID NO: 15), and 13 (SEQ ID NO: 13) show less than 20% homology. Ig-fold sequences were predicted using the SMART data bank. (FIG. 11B) Extracellular ectodomains of Merlin RL12, RL13, gp34, and gp68 with c-term FLAG tags and PDGFRα transmembrane domain were expressed on ExpiCHO. Cells were stained with 300 nM hu4D5 with WT human IgG1 Fc+/−3 μM soluble gp34-M and binding was detected with anti-huFcγ-AF647 and anti-FLAG(M2)-PE secondaries using flow cytometry. Double positives (PE/AF647) were gated and the GMFI of antibody binding (AF647) was plotted. (FIG. 11C) vFcγR ectodomains expressed on ExpiCHO cells were also stained with hu4D5 Fc variants (WT, G2, and G5) in a dose dependent manner, with binding detected using goat-anti-Fcγ-AF647 and 4PL curves fit using GraphPad. Non-transfected cells were used as a specificity control. For both (B) and (C), mean fluorescence intensity±SD is plotted and one-way ANOVA with Tukey test multiple comparisons was used, *p<0.05, **p<0.01, ***p<0.001, ****p<0.0001, ns: non-significant. Representative data of one experiment shown, and each experiment was repeated at least twice.

FIGS. 12A-C. ADCC and ADCP activities of all selected Fc variants. (FIG. 12A) ADCC assay with SKOV3 cells incubated with NK-92 V158 or NK-92 F158 haplotypes in the presence of hu4D5 Fc variants. % ADCC values for variants compared to WT-IgG1 were not statistically significant except for R47, YTE, and isotype (p<0.05). For the V158 all variants had greater overall ADCC (p<0.001), except isotype. (FIG. 12B) ADCP assay was performed using phrodo-Green/APC-polystyrene beads coated with post-fusion gB. Beads were incubated in the presence of anti-gB antibody, 27-287 or SM5-1, in the presence of THP1 monocytes (50 beads per cell). Phagocytosis score was calculated as the percent positive of APC/FITC cells multiplied by the GMFI of APC. (FIG. 12C). ADCP was done in the presence of 27-287 Fc variants and compared to WT were not statistically significant except for G2 and isotype (p<0.001). Representative data of one experiment is shown and each experiment was repeated at least twice. Data are mean±SD and statistical analysis was performed using two-way ANOVA followed by Tukey's multiple comparison test, *p<0.05, **p<0.01, ***p<0.001, ****p<0.0001, ns: non-significant.

FIGS. 13A-B. CD16A activation with SM5-1 Fc variants in the presence of AD169 infected cells. (FIG. 13A) BW-CD16A-ζ reporter cells were incubated with AD169 wild-type or vFcγR deleted (ΔΔΔ) infected fibroblasts (MOI=5, 72 hpi) in the presence of SM5-1 Fc variants (100-0.07 μg/mL; 2 fold dilutions). CD16A signaling measured by mouse IL-2 (m-IL2) secretion and the dose-response curves represent the average of 3 independent experiments and were fit to 4PL or AUC using GraphPad. Mean EC₅₀ values (μg/mL) for each mAb or combination are shown; ND indicates activation was not detected at 100 μg/mL. p values indicate differences in EC₅₀ for mAb's activation in the presence of AD169 and ΔΔΔ. Asterisks next to EC₅₀ values correlate to comparison between each variant to SM5-1 WT. The AUC was normalized to SM5-1 WT activation with ΔΔΔ infection. Data is shown as the mean±SD of three independent experiments. (FIG. 13B) Percentage of NK-92 (V or F158) degranulation (CD107A) in the presence of AD169 infected fibroblasts (MOI=2, 96 hpi) with SM5-1 Fc variants detected using flow cytometry (anti-CD107A-APC). Data are presented as mean±SD (n=2) and statistical analysis was performed using two-way ANOVA with Tukey's multiple comparisons test. *p<0.05, **p<0.01, ***p<0.001, ****p<0.0001, ns=non-significant, unless stated otherwise for all data sets.

FIGS. 14A-E. Fc variants G2 and G2E both lose considerable binding to gp34 and gp68 as compared to WT Fc. (FIG. 14A) Binding of antibodies comprising the SM5-1 anti-gB binding site and Fc variants to gB antigen was evaluated by ELISA. (FIG. 14B) Binding of antibodies comprising the SM5-1 anti-gB binding site and Fc variants to purified gp34 was evaluated by ELISA. (FIG. 14C) Binding of antibodies comprising the SM5-1 anti-gB binding site and Fc variants to purified gp68 was evaluated by ELISA. (FIG. 14D) Expression of HSV-1 gE/gI protein on the ExpiCHO surface confirmed by staining with commercial anti-gE antibody. Stained with 2B1 mFc or anti-gE mFc at 5 μg/mL followed by 1:500 dilution of Goat anti-mFc AF647. Non-specific binding to untransfected cells<1%. (FIG. 14E) Binding of Fc variants to HSV-1 gE/gI viral Fc receptor expressed on ExpiCHO cells.

FIGS. 15A-B. Fc variants G2 and G2E retain binding to the host Fc receptors CD16a and FcRn as compared to WT Fc. (FIG. 15A) Binding of antibodies comprising the SM5-1 anti-gB binding site and Fc variants to CD16A was evaluated by ELISA. (FIG. 15B) Binding of antibodies comprising the SM5-1 anti-gB binding site and Fc variants to FcRn was evaluated by ELISA.

FIGS. 16A-C. Characterization of G2E Fc. (FIG. 16A) Serum antibody concentration in homozygous Tg32 transgenic mice (n=3-4) as a function of time after administration. Each hu4D5 Fc variant (WT, G2, G5, G2E, IHH) was administered intraperitoneally at 2 mg/kg, with sera antibody concentrations determined by antigen specific ELISA. Data are presented as mean±SD (n=3-4). (FIG. 16B) Data for clearance of G2E from each replicate mouse from FIG. 16A. (FIG. 16C) Thermal stability was measured using differential scanning fluorimetry. In the left graph, the lines represent, from top to bottom at 60° C., G2, G2E, and WT.

FIGS. 17A-C. Binding of anti-gp34 antibodies to cells expressing gp34. gp34-FLAG with a PDGFR anchor was transiently expressed on the surface of ExpiCHO cells. (FIG. 17A) gp34 expressed on the surface of ExpiCHO cells. Untransfected and gp34-FLAG-expressing ExpiCHO cells were stained using an anti-FLAG PE antibody and fluorescence was detected using flow cytometry. (FIGS. 17B-C) Cells were then stained with anti-gp34 antibodies made with mouse Fcs domains followed by anti-mouse Fc AF647: FIG. 17B, Untransfected and FIG. 17C, gp34-FLAG-expressing ExpiCHO cells and binding evaluated with flow cytometry.

### DETAILED DESCRIPTION

Development of HCMV vaccines has been hindered by inefficient neutralization of cell-to-cell spread and poor NK cell clearance of infected cells. HCMV immune evasion strategies include those that specifically undermine anti-HCMV antibodies. Four viral Fc receptors (vFcγR) are expressed on the surface of infected cells and virions. These exhibit distinct Fc binding activities and antagonize Fc interactions with host effector FcγRs (Corrales-Aguilar et al., 2014a, Corrales-Aguilar et al., 2014b). The presence of all four vFcγRs is conserved in HCMV clinical isolates, with genes RL11 and UL118-UL119 (encoding gp34 and gp68, respectively) showing little sequence variation, while RL12 and RL13 are highly divergent (Corrales-Aguilar et al., 2014a), and RL13 shows no functional expression during in vitro virus cultivation. The two conserved vFcγRs, gp34 and gp68, bind independent epitopes on the Fc, co-operatively internalize antibodies and antibody/viral glycoprotein complexes to clear the infected cell surface and inhibit antibody-dependent CD16A+NK cell activation (Kolb et al., 2021). The alpha-herpes viruses, including herpes simplex 1 and 2 and varicella zoster, each express a single vFcγR, the gE-gI heterodimer, which internalizes antibodies on the surface of infected cells and inhibits host FcγR activation (Jenks et al., 2019). Notably, the varicella zoster gE protein serves as the basis of a successful shingles vaccine (Shingrix, GSK) (Heineman et al., 2019).

HCMV expresses viral Fcγ receptors (vFcγRs) on infected cells, which bind human IgG Fc domains, leading to dampened CD16A+NK-cell responses and antibody targeting to the lysosome. Fcs resisting capture by these receptors could mediate potent anti-viral effects. As such, binding to two highly conserved vFcγRs, gp34 and gp68, which compete for Fc binding with CD16A and FcRn, respectively, was characterized. Fc variants that retain physiological affinities for CD16A and FcRn but exhibit markedly reduced affinities for gp34 and gp68 were engineered. Additionally, some Fc variants lose affinity for gpRL12, gpRL13 and the gE/gI of alpha herpes viruses without altering affinity for the host receptors CD16A and FcRn. Anti-gB antibodies bearing vFcγR-resistant Fc domains were inefficiently internalized and mediated greatly enhanced CD16A activation against HCMV-infected fibroblasts. These data are the first to demonstrate strong CD16A+NK cell activation against the leading gB vaccine candidate and suggest novel strategies for immune control of HCMV infection.

### I. Aspects of the Present Disclosure

An on-going challenge in development of HCMV vaccines and therapeutics is the unclear role of antibody-mediated cellular immune responses in protection. While strong correlations have been observed between non-neutralizing anti-gB antibodies and protection in vaccine trials (Jenks et al., 2019, Nelson et al., 2018) and there is evidence suggesting that ADCC, ADCP, and complement can contribute to protection (Li et al., 2017, Nelson et al., 2018, Vietzen et al., 2020), in vitro cellular responses with immune sera are generally weak. Similarly, neutralizing monoclonal antibodies have been prioritized for development since in vitro ADCC is only observed under limited circumstances (Vlahava et al., 2021, Vietzen et al., 2020). Cellular responses are increasingly shown to play important roles in clearance of infected cells, especially for cell-associated viruses like HCMV, with Fc modifications increasing the potency of antibodies targeting Ebola, HIV, and HCMV (Rossignol et al., 2021, Gunn et al., 2021, Vietzen et al., 2020, Van den Hoecke et al., 2017). Hypothesizing that antibody capture by the four HCMV vFcγRs undermines antibody-mediated cellular responses against HCMV, studies were performed to better define Fc-vFcγR interactions and harness this knowledge to engineer human IgG1 Fc variants that resist vFcγR capture to potently activate CD16A responses against infected cells.

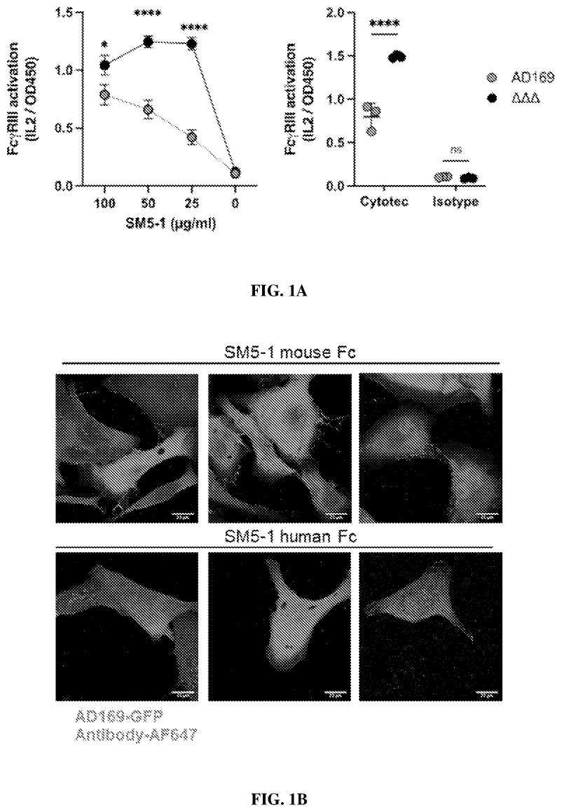

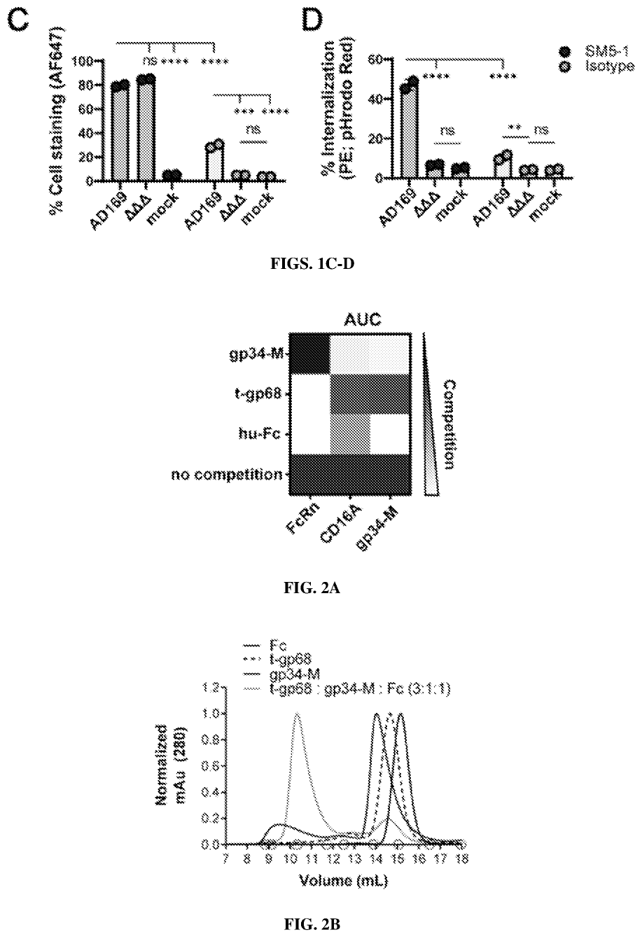

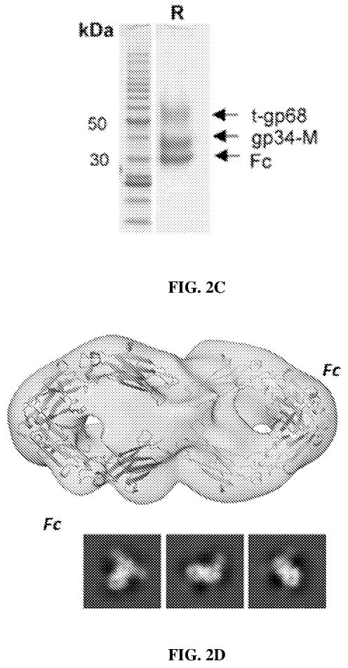

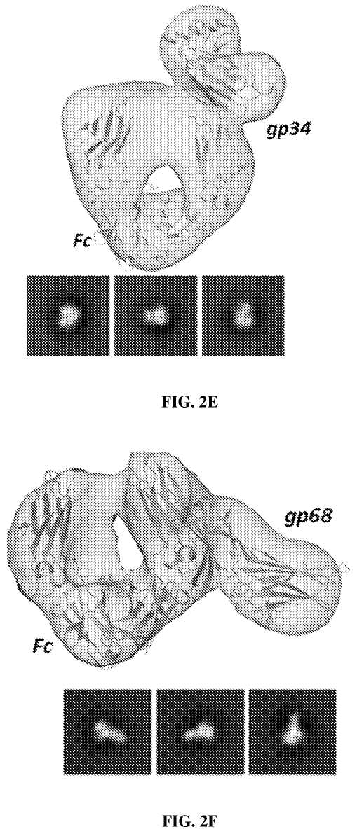

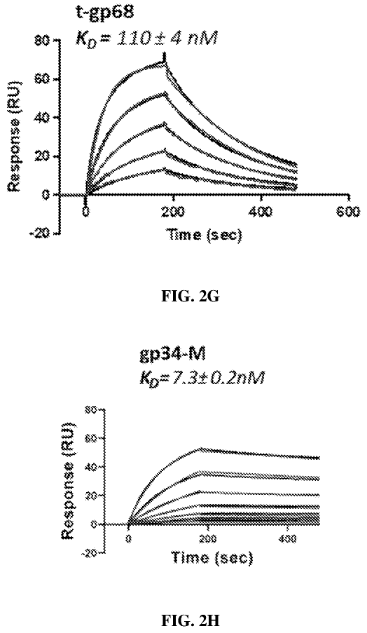

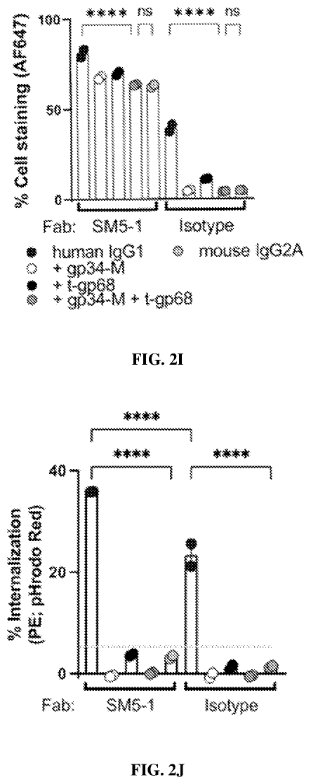

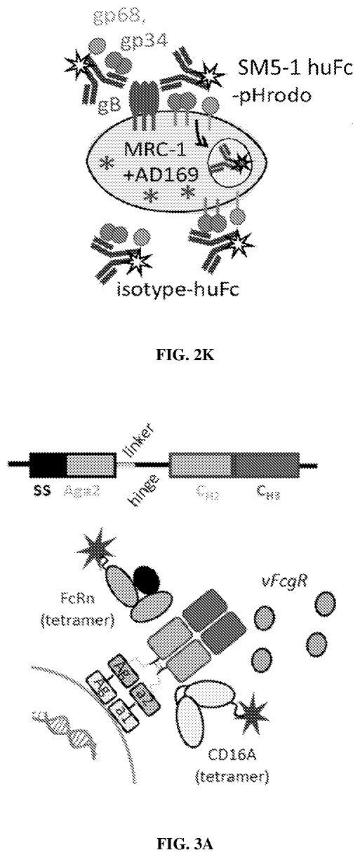

These studies focused primarily on the vFcγR receptors gp34 and gp68, which are highly conserved across all clinical HCMV isolates. These were previously reported to co-operatively capture antibodies via the Fc domain: gp68 by clustering antibodies on the infected cell surface and gp34 by mediating antibody cellular internalization (Kolb et al., 2021). Using engineered soluble receptor ectodomains, nsEM and competition ELISAs, gp34 was found to inhibit CD16A/Fc binding while gp68 was found to inhibit FcRn/Fc binding. Using SPR, both receptors were found to bind human IgG1 Fc with high affinities (7 nM K_{d} for gp34; 70-110 nM K_{d} for gp68; FIGS. 2G,H). These data confirm the previously anticipated gp68 binding site and affinity (Sprague et al., 2008) while providing new insights into gp34/Fc interactions. gp34 and gp68 selectively capture antibodies bound to antigens on an infected cell: antibodies binding gB were internalized more efficiently than an isotype control and this process was inhibited by genetic deletion of all vFcγRs (FIGS. 1B-C) or by competition with high concentrations (>10×K_{d}) of soluble gp34 and gp68, individually and in combination (FIGS. 2I,J). The 10-fold stronger gp34/Fc affinity suggests that gp34 may compete with host FcγR more effectively than gp68 to capture antibody Fc domains, depending on vFcγR expression level and sub-cellular location.

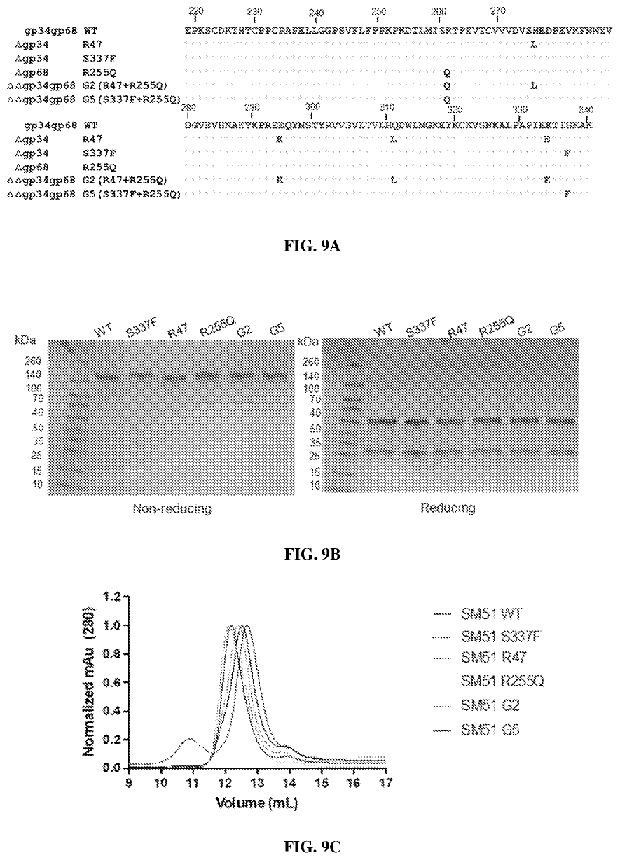

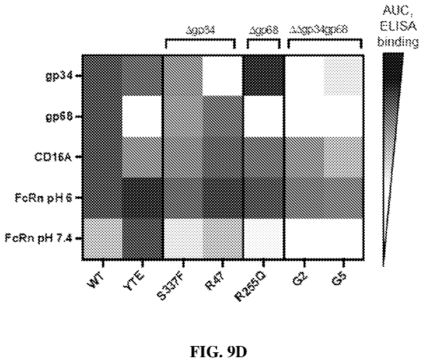

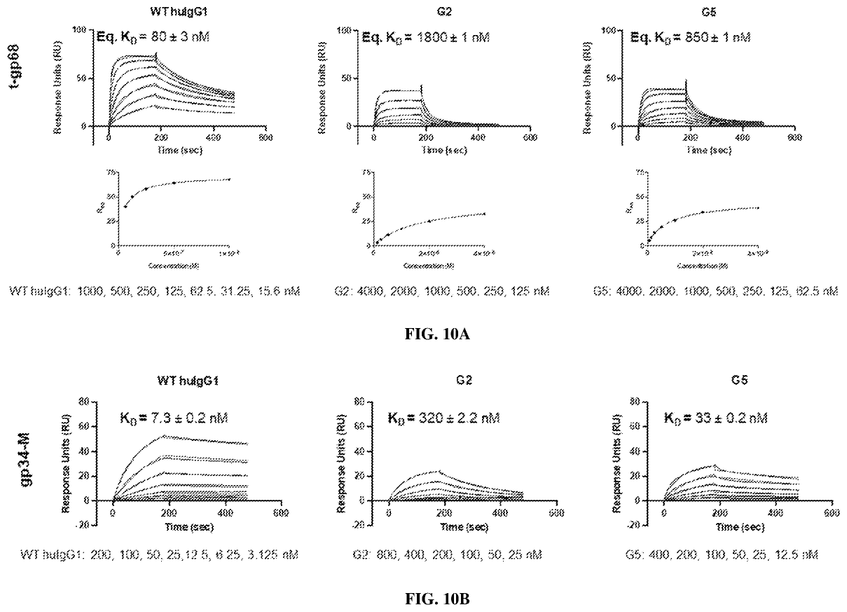

Using yeast display and a competitive staining strategy, two libraries of randomized Fc domains were created and variants that maintain binding to FcRn and CD16A but have greatly reduced affinities for gp34 and gp68 were identified. This resulted in identification of variant G2 containing five residue changes (R255Q, H268L, E294K, Q311L, K334E) that confer ˜50-70-fold reduced gp34 and gp68 affinities and variant G5 containing two residue changes (R255Q and S337F) that confer ˜5 and 20-fold reduced gp34 and gp68 binding, respectively (Table 1). These two Fc variants retain physiological affinities for FcRn and CD16A, although their thermal stability and pharmacokinetic properties were reduced compared to wild-type Fc. Residue changes selected for gp34-resistance primarily localize to the upper CH₂ region and overlap with the CD16A and gp34 binding sites, while changes selected for gp68-resistance localize to the CH₂—CH₃ interface expected for the FcRn and gp68 interfaces (FIG. 9).

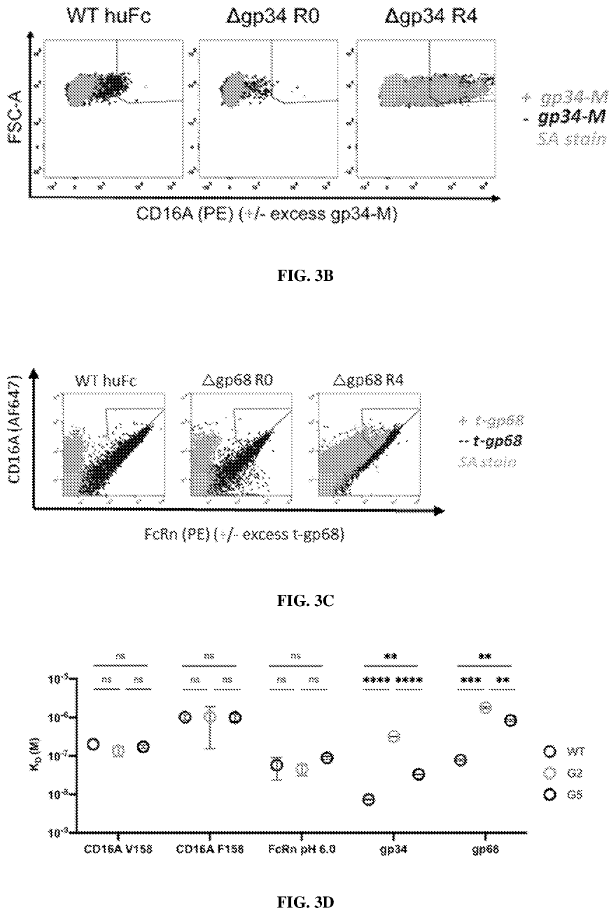

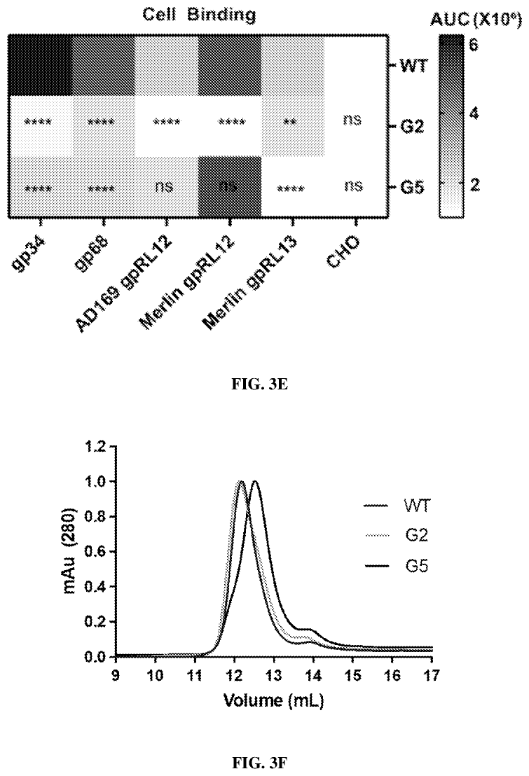

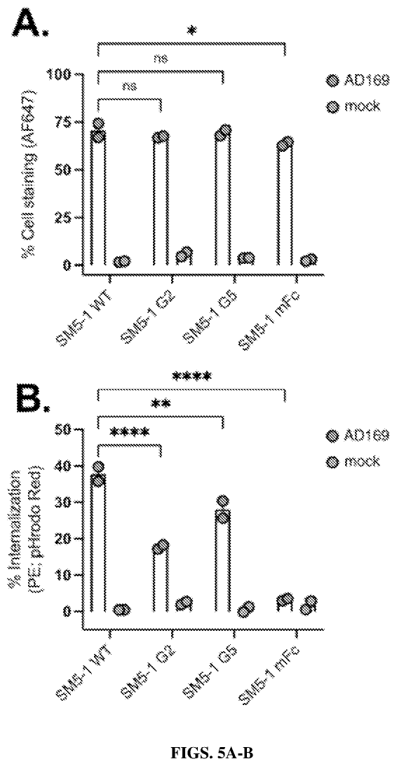

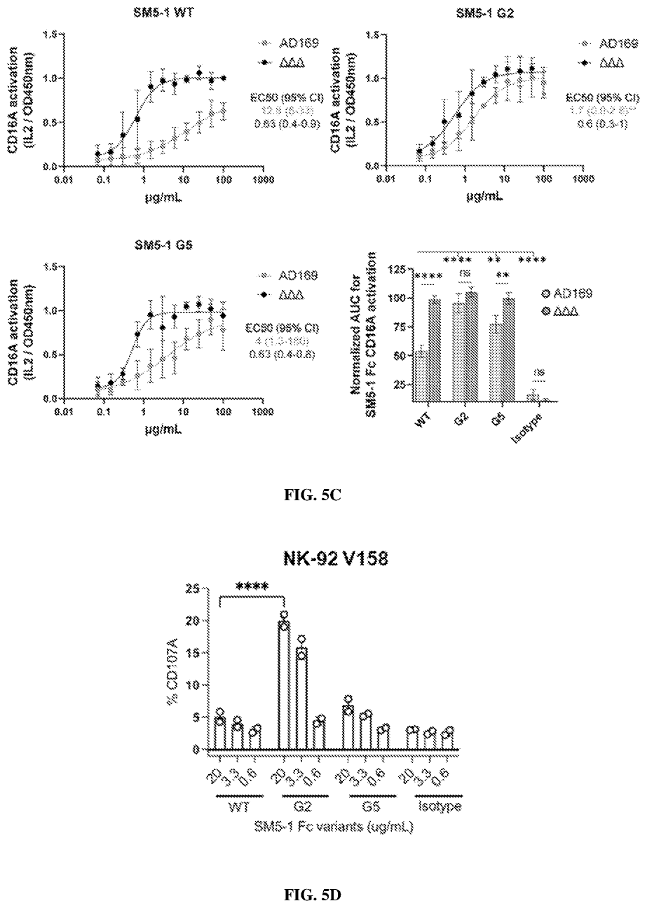

HCMV is unique among herpesviruses as the only species coding for more than one vFcγR (Corrales-Aguilar et al., 2014a). The need for this redundancy is unclear, but prior reports suggest each vFcγR employs distinct mechanisms of Fc capture, based on Fc binding footprint, sub-cellular location and downstream signaling effects (Corrales-Aguilar et al., 2014a). Fc variant G2, which has the greatest affinity loss for gp34 and gp68, also shows the greatest potency in three in vitro assays. In these studies, Fc variants with reduced gp34 binding (e.g., G2) better resist internalization by infected cells and recover Fc functions to a greater extent than Fc domains with reduced gp68 binding (FIG. 5B), although Fc variants with reduced binding to a single vFcγR (e.g., S337F, R47, R255Q) highlight the importance of both gp68 and gp34 to CD16A antagonization (FIGS. 5F,13A,13B). Since G2 but not G5 also lost affinity for vFcγR gpRL12 (FIGS. 3D,E), this vFcγR may contribute to G2's enhanced potency. The partial loss of binding to gpRL12 by G2 and gpRL13 by G5 (FIG. 3E) suggests that Fc variants that selectively lose binding to a single RL11-family member could be generated and used to delineate the contributions of each vFcγR to HCMV antibody escape.

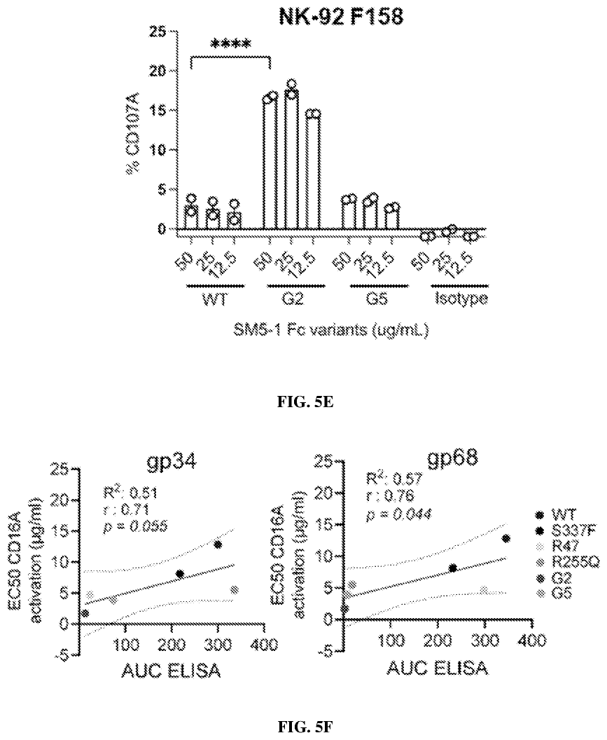

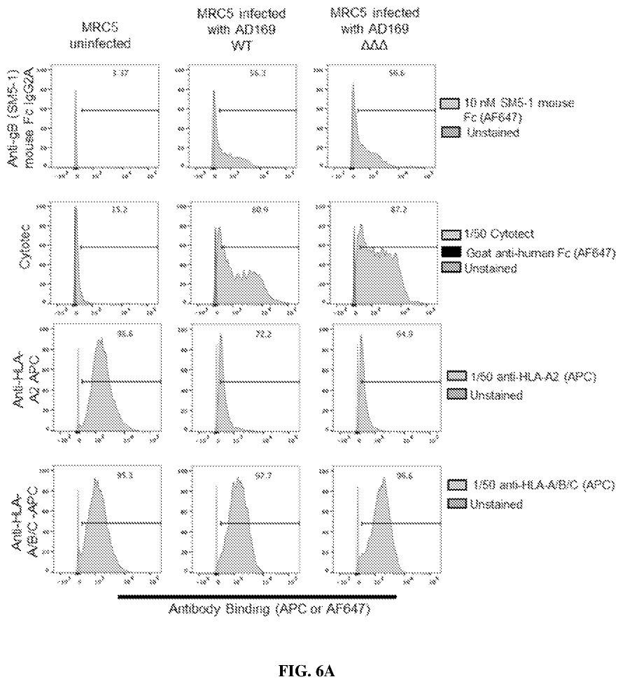

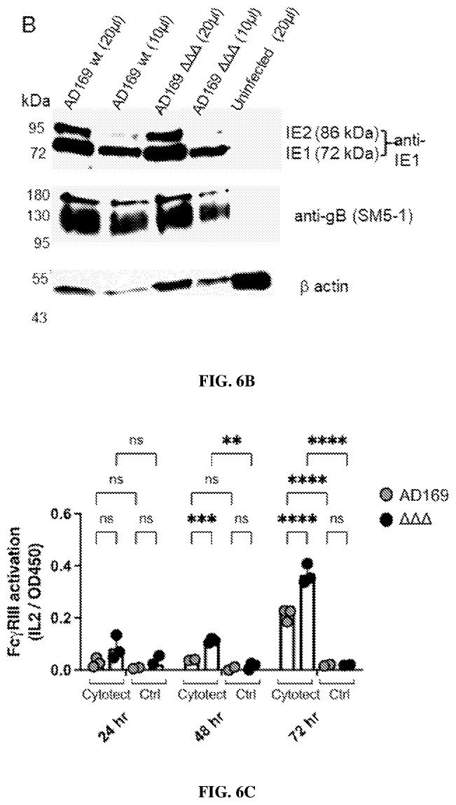

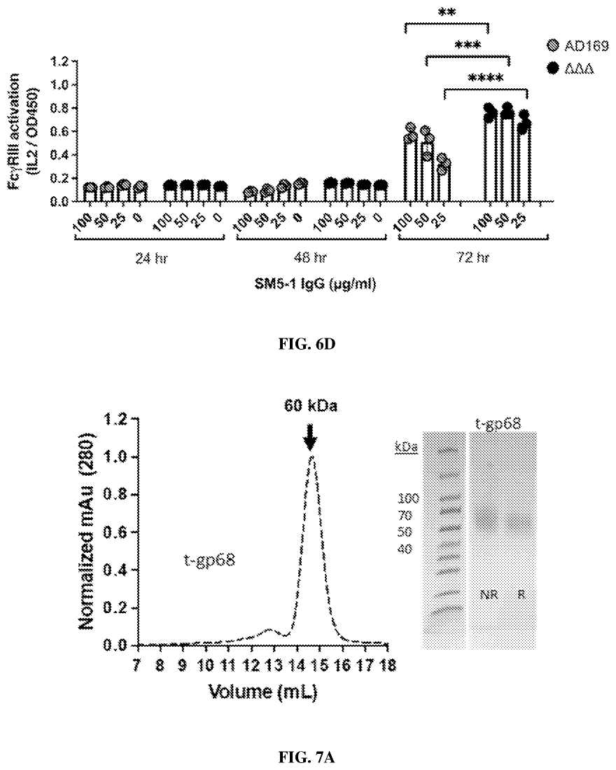

Experiments with influenza have demonstrated that some antigens and epitopes better activate cellular immune responses including ADCC (He et al., 2016). While the presence of antibodies binding native gB are predictive of vaccine efficacy in humans (Jenks et al., 2020) and gB-specific antibodies accounted for the majority of the ADCC response in an HCMV-seropositive lung transplant study (Vietzen et al., 2017, Vietzen et al., 2020), the impact of vFcγR activities on these responses was previously unknown. The function of Fc variants was tested in the context of infected cells with the monoclonal antibody SM5-1, which binds antigenic domain 4 on pre- and post-fusion gB conformations and strongly binds gB on the surface of HCMV infected cells (Liu et al., 2021) (FIGS. 6A-B). Deletion of the vFcγRs or use of a vFcγR-resistant Fc significantly increased CD16A activation by SM5-1 (FIGS. 1A,6D) and reduced its internalization by infected cells (FIGS. 1D,5B). Since gB has five antigenic domains, the vFcγR-resistant Fc variants described here will support identification of ADCC-inducing protective gB epitopes which could be masked by vFcγR expression.

The inventors also focused on enhancing NK cell degranulation as HCMV-infected cells are resistant to lysis (Wu et al., 2015b). CD16A activation by antibodies can overcome inhibitory NK cell signaling pathways (Forrest et al., 2020) while release of IFNγ can suppress virion production and cell-to-cell spread and IL-2 enhances T-cell expansion (Wu et al., 2015b). Remarkably, nearly identical CD16A activation profiles were observed for cells infected with AD169 or the isogenic vFcγR-deficient strain ΔΔΔ when SM5-1 was combined with the vFcγR-resistant G2 Fc (FIG. 5C). Similarly, NK cell degranulation increased >3-fold for infected fibroblasts treated with SM5-1 with the G2 versus wild-type Fc, regardless of the CD16A allele present (FIG. 5D). A recent report examined the potential for Fc domains with enhanced Fc/CD16A affinity to overcome vFcγR antagonism (Vlahava et al., 2021). Antibodies binding HCMV glycoproteins UL16 and UL141 were modified to include the S239D and 1332E residues which increase Fc/CD16A affinity by 100-fold (Lazar et al., 2006). When used in in vitro NK cell degranulation assays, a ˜3-fold increase was observed for modified versus wild-type IgG1 when multiple antibodies binding the same antigen were pooled (Vlahava et al., 2021). Fc modifications to reduce vFcγR binding may provide a wider therapeutic window to achieve similar CD16A activation towards a clinically relevant gB epitope. This approach can protect binding to other FcγRs and thereby preserve ADCP and complement activities, in addition to ADCC.

HCMV vaccine development efforts have focused on the major viral glycoproteins, which mediate virion attachment (gH/gL dimer in tri- and pentameric complexes) and invasion of host cells (gB fusogen), since blocking these steps can prevent infection. Viral glycoproteins are among the most highly expressed on the infected cell surface (Vlahava et al., 2021) and exhibit synchronized expression kinetics with vFcγRs (FIGS. 6C-D), suggesting vFcγRs specifically target the major HCMV glycoproteins and will undermine efforts to develop vaccines and therapeutic antibodies. Future vaccines may therefore benefit from inclusion of antigens that are expressed at earlier timepoints and are minimally affected by vFcγR activities. In addition, the similarities between the HCMV vFcγRs and the alphaherpes virus homolog gE, which forms the basis of a highly effective shingles subunit vaccine, supports the potential use of vFcγRs as vaccine antigens.

Provided herein are human IgG1 Fc variants with greatly reduced affinities for two highly conserved HCMV vFcγRs. Consistent with the hypothesis that resistance to vFcγR capture will enhance anti-viral antibody activities, anti-gB antibodies bearing Fc modified domains mediated potent CD16A-signalling against HCMV-infected cells. A major limitation in development of HCMV therapeutics is the lack of a predictive animal model allowing for infection with HCMV; however, rhesus macaques and RhCMV are experimentally tractable. This strain expresses a single vFcγR: Rh05 is a member of the RL11 gene family and shares several key characteristics with gp34, including Fc binding and antagonism of rhesus Fc receptors in vitro (Kolb et al., 2019). Since human IgG1 exhibits cross-reactivity with rhesus FcRs (Boesch et al., 2017), future efforts will explore the use of this model to provide in vivo insight into the impact of vFcγR-resistant antibodies. Antibodies engineered to resist capture by HCMV vFcγRs are expected to address the limitations of prior therapeutic HCMV antibodies to result in improved viral clearance and clinical outcomes.

Herpes simplex virus (HSV)1, HSV2 or varicella zoster (VZV) gE can form a noncovalent heterodimer complex with HSV1, HSV2, or VZV (respectively) glycoprotein I (gI). The gE/gI heterodimer functions as a viral Fc gamma receptor (FcγR), meaning it has the capacity to interact with the Fc portion of human IgG. Indeed, HSV1 or HSV2 gE or gE/gI heterodimer, when displayed at the cell surface of HSV infected cells, bind host IgG through their Fc portion. In addition, a crystal structure of HSV gE/gI has shown that it binds to Fc via the C-terminal residues 235-380 in a pH-selective manner. And in fact, the human IgG1 Fc variants provided herein also display reduced affinities for the HSV homolog to gp68, i.e., HSV gE/gI. HSV-1, HSV-2 and varicella zoster all express gE/gI homologs. As such, the modified Fc variants also resist capture by Fc receptors generated by multiple herpes-virus species. Since the VZV gE/gI complex exhibits high homology to the HSV gE/gI, it is expected these Fc variants will also lose affinity for VSV gE/gI.

### II. Definitions

“Nucleic acid,” “nucleic acid sequence,” “oligonucleotide,” “polynucleotide” or other grammatical equivalents as used herein means at least two nucleotides, either deoxyribonucleotides or ribonucleotides, or analogs thereof, covalently linked together. Polynucleotides are polymers of any length, including, e.g., 20, 50, 100, 200, 300, 500, 1000, 2000, 3000, 5000, 7000, 10,000, etc. A polynucleotide described herein generally contains phosphodiester bonds, although in some cases, nucleic acid analogs are included that may have at least one different linkage, e.g., phosphoramidate, phosphorothioate, phosphorodithioate, or O-methylphophoroamidite linkages, and peptide nucleic acid backbones and linkages. Mixtures of naturally occurring polynucleotides and analogs can be made; alternatively, mixtures of different polynucleotide analogs, and mixtures of naturally occurring polynucleotides and analogs may be made. The following are non-limiting examples of polynucleotides: a gene or gene fragment, exons, introns, messenger RNA (mRNA), transfer RNA, ribosomal RNA, ribozymes, cDNA, cRNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, the term polynucleotide encompasses both the double-stranded form and each of two complementary single-stranded forms known or predicted to make up the double-stranded form. A polynucleotide is composed of a specific sequence of four nucleotide bases: adenine (A), cytosine (C), guanine (G), thymine (T), and uracil (U) for thymine when the polynucleotide is RNA. Thus, the term “polynucleotide sequence” is the alphabetical representation of a polynucleotide molecule. Unless otherwise indicated, a particular polynucleotide sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues.

The terms “peptide,” “polypeptide” and “protein” used herein refer to polymers of amino acid residues. These terms also apply to amino acid polymers in which one or more amino acid residues is an artificial chemical mimetic of a corresponding naturally occurring amino acid, as well as to naturally occurring amino acid polymers, those containing modified residues, and non-naturally occurring amino acid polymers. In the present case, the term “polypeptide” encompasses an antibody or a fragment thereof.

Other terms used in the fields of recombinant nucleic acid technology, microbiology, immunology, antibody engineering, and molecular and cell biology as used herein will be generally understood by one of ordinary skill in the applicable arts.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the inherent variation in the method being employed to determine the value, the variation that exists among the study subjects, or a value that is within 10% of a stated value.

### III. Antibodies and Modifications of Antibodies

Provided herein are antibodies having modified IgG1 Fc domains that have reduced affinity for viral FcγRs, including HCMV gp34, HCMV gp68, HCMV gpRL12, HCMV gpRL13, HSV-1 gE/gI, HSV-2 gE/gI, and VZV gE/gI, while maintaining physiological affinity for CD16A and FcRn, as compared to a wild-type IgG1 Fc domain. The variable regions of these antibodies may target HCMV antigens, such as HCMV glycoproteins, e.g., gH, gL, gB, gO, gN, gM, UL83, UL123, UL128, UL130 and UL131A, or pp65. Exemplary antibodies that have variable regions that bind to HCMV antigens can be found, for example, in U.S. Pat. No. 9,346,874; Spindler et al., PLoS Pathog., 10:31004377, 2014. Such antibodies may be produced using methods described herein.

Provided herein are antibodies or antibody fragments comprising a human IgG1 heavy chain Fc domain comprising an engineered version of the following amino acid sequence:

(SEQ ID NO: 1)

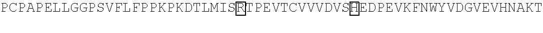

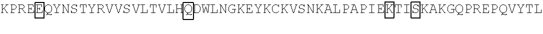

PPSRDELTKNQVSLTCLVKGFYPSDIAVEWESNGQPENNYKTTPPVLDSDGSFFLYSKLTVD
KSRWQQGNVFSCSVMHEALHNHYTQKSLSLSPGK.

In some aspects, the Fc domain is modified at amino acid Arg255 (boxed in SEQ ID NO: 1 in the preceding paragraph) to reduce affinity for viral FcγRs, such as HCMV gp34 and gp68, for example Arg255Gln (R255Q). In some aspects, the Fc domain is modified at amino acid His268 (boxed in SEQ ID NO: 1 in the preceding paragraph) to reduce affinity for viral FcγRs, such as HCMV gp34 and gp68, e.g., His268Leu (H268L). In some aspects, the Fc domain is modified at amino acid Glu294 (boxed in SEQ ID NO: 1 in the preceding paragraph), e.g., Glu294Lys (L294K). In some aspects, the Fc domain is altered at amino acid Gln311 (boxed in SEQ ID NO: 1 in the preceding paragraph), e.g., Gln311Leu (Q311L). In some aspects, the Fc domain is altered at amino acid Lys334 (boxed in SEQ ID NO: 1 in the preceding paragraph), e.g., Lys334Glu (K334E). In some aspects, the Fc domain is altered at amino acid Ser337 (boxed in SEQ ID NO: 1 in the preceding paragraph), e.g., Ser337Phe (S337F). All residue numbers are according to EU numbering (Kabat, E. A., et al. (1991) SEQUENCES OF PROTEINS OF IMMUNOLOGICAL INTEREST, FIFTH EDITION, U.S. Department of Health and Human Services, NIH Publication No. 91-3242).

Provided herein are antibodies or antibody fragments comprising a human IgG1 heavy chain Fc domain comprising an engineered version of the following amino acid sequence:

(SEQ ID NO: 2)

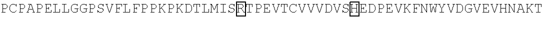

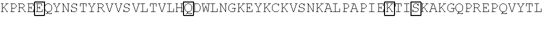

PPSREEMTKNQVSLTCLVKGFYPSDIAVEWESNGQPENNYKTTPPVLDSDGSFFLYSKLTVD
KSRWQQGNVFSCSVMHEALHNHYTQKSLSLSPGK.

In some aspects, the Fc domain is modified at amino acid Arg255 (boxed in SEQ ID NO: 1 in the preceding paragraph) to reduce affinity for viral FcγRs, such as HCMV gp34 and gp68, for example Arg255Gln (R255Q). In some aspects, the Fc domain is modified at amino acid His268 (boxed in SEQ ID NO: 1 in the preceding paragraph) to reduce affinity for viral FcγRs, such as HCMV gp34 and gp68, e.g., His268Leu (H268L). In some aspects, the Fc domain is modified at amino acid Glu294 (boxed in SEQ ID NO: 1 in the preceding paragraph), e.g., Glu294Lys (L294K). In some aspects, the Fc domain is altered at amino acid Gln311 (boxed in SEQ ID NO: 1 in the preceding paragraph), e.g., Gln311Leu (Q311L). In some aspects, the Fc domain is altered at amino acid Lys334 (boxed in SEQ ID NO: 1 in the preceding paragraph), e.g., Lys334Glu (K334E). In some aspects, the Fc domain is altered at amino acid Ser337 (boxed in SEQ ID NO: 1 in the preceding paragraph), e.g., Ser337Phe (S337F). All residue numbers are according to EU numbering (Kabat, E. A., et al. (1991) SEQUENCES OF PROTEINS OF IMMUNOLOGICAL INTEREST, FIFTH EDITION, U.S. Department of Health and Human Services, NIH Publication No. 91-3242).

Provided herein are antibodies or antibody fragments comprising a modified human IgG1 heavy chain Fc domain comprising the following amino acid sequence:

(SEQ ID NO: 3)

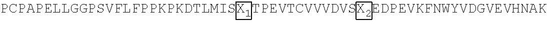

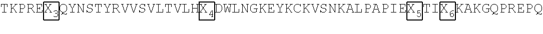

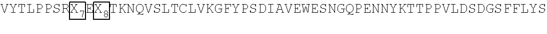

KLTVDKSRWQQGNVFSCSVMHEALHNHYTQKSLSLSPGK,

wherein X1 is Arg or Gln; X₂ is His or Leu; X₃ is Glu or Lys; X₄ is Gln or Leu; X₅ is Lys or Glu; X₆ is Ser or Phe; X₇ is Asp or Glu; X₈ is Leu or Met.

Provided herein are antibodies or antibody fragments comprising a modified human IgG1 heavy chain Fc domain comprising the following amino acid sequence:

(SEQ ID NO: 4)
PCPAPELLGGPSVFLFPPKPKDTLMISQTPEVTCVVVDVSLEDPEVKFNWYVDGVEVHNAKT
KPREKQYNSTYRVVSVLTVLHLDWLNGKEYKCKVSNKALPAPIEETISKAKGQPREPQVYTL

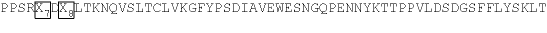

VDKSRWQQGNVFSCSVMHEALHNHYTQKSLSLSPGK,

wherein X₇ is Asp or Glu; and X₈ is Leu or Met.

Provided herein are antibodies or antibody fragments comprising a modified human IgG1 heavy chain Fc domain comprising the following amino acid sequence:

(SEQ ID NO: 5)
PCPAPELLGGPSVFLFPPKPKDTLMISQTPEVTCVVVDVSHEDPEVKFNWYVDGVEVHNAKT
KPREEQYNSTYRVVSVLTVLHQDWLNGKEYKCKVSNKALPAPIEKTIFKAKGQPREPQVYTL

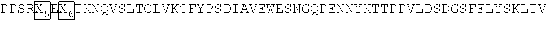

DKSRWQQGNVFSCSVMHEALHNHYTQKSLSLSPGK,

wherein X₇ is Asp or Glu; and X₈ is Leu or Met.

Provided herein are antibodies or antibody fragments comprising a modified human IgG1 heavy chain Fc domain comprising the following amino acid sequence:

(SEQ ID NO: 28)
PCPAPELLGGPSVFLFPPKPKDTLMISQTPEVTCVVVDVSLEDPEVKFNWYVDGVEVHNAKT
KPREEQYNSTYRVVSVLTVLHLDWLNGKEYKCKVSNKALPAPIEETISKAKGQPREPQVYTL

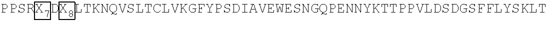

VDKSRWQQGNVFSCSVMHEALHNHYTQKSLSLSPGK,

wherein X₇ is Asp or Glu; and X₈ is Leu or Met.

Provided herein are antibodies or antibody fragments comprising a modified human IgG1 heavy chain Fc domain comprising the following amino acid sequence:

(SEQ ID NO: 30)
PCPAPELLGGPSVFLFPPKPKDTLMISQTPEVTCVVVDVSLEDPEVKFNWYVDGVEVHNAKT
KPREEQYNSTYRVVSVLTVLHLDWLNGKEYKCKVSNKALPAPIEETIFKAKGQPREPQVYTL

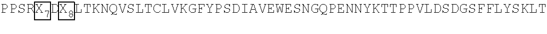

VDKSRWQQGNVFSCSVMHEALHNHYTQKSLSLSPGK,

wherein X₇ is Asp or Glu; and X₈ is Leu or Met.

Provided herein are antibodies or antibody fragments comprising a modified human IgG1 heavy chain Fc domain comprising the following amino acid sequence:

(SEQ ID NO: 6)
EPKSCDKTHTCPPCPAPELLGGPSVFLFPPKPKDTLMISQTPEVTCVVVDVSLEDPEVKENW
YVDGVEVHNAKTKPREKQYNSTYRVVSVLTVLHLDWLNGKEYKCKVSNKALPAPIEETISKA
KGQPREPQVYTLPPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK.

Provided herein are antibodies or antibody fragments comprising a modified human IgG1 heavy chain Fc domain comprising the following amino acid sequence:

(SEQ ID NO: 7)
EPKSCDKTHTCPPCPAPELLGGPSVFLFPPKPKDTLMISQTPEVTCVVVDVSHEDPEVKFNW
YVDGVEVHNAKTKPREEQYNSTYRVVSVLTVLHQDWLNGKEYKCKVSNKALPAPIEKTIFKA
KGQPREPQVYTLPPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK.

Provided herein are antibodies or antibody fragments comprising a modified human IgG1 heavy chain Fc domain comprising the following amino acid sequence:

(SEQ ID NO: 29)
EPKSCDKTHTCPPCPAPELLGGPSVFLFPPKPKDTLMISQTPEVTCVVVDVSLEDPEVKFNW
YVDGVEVHNAKTKPREEQYNSTYRVVSVLTVLHLDWLNGKEYKCKVSNKALPAPIEETISKA
KGQPREPQVYTLPPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK.

Provided herein are antibodies or antibody fragments comprising a modified human IgG1 heavy chain Fc domain comprising the following amino acid sequence:

(SEQ ID NO: 31)
EPKSCDKTHTCPPCPAPELLGGPSVFLFPPKPKDTLMISQTPEVTCVVVDVSLEDPEVKFNW
YVDGVEVHNAKTKPREEQYNSTYRVVSVLTVLHLDWLNGKEYKCKVSNKALPAPIEETIFKA
KGQPREPQVYTLPPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK.

In some aspects, the human IgG1 constant region is modified to comprise either a “knob” mutation, e.g., T366Y, or a “hole” mutation, e.g., Y407T, for heterodimerization with a second constant region (residue numbers according to EU numbering (Kabat, E. A., et al., supra)).

In some aspects, the constant region of the heavy chain of the antibody is a human IgG1 isotype, e.g., an allotype of the human IgG1 isotype, e.g., the IgG1 G1m3 allotype. Exemplary human IgG1 allotypes are described in Magdelaine-Beuzelin et al. (2009) PHARMACOGENET. GENOMICS 19(5):383-7.

In some aspects, the human IgG constant region is modified to enhance FcRn binding. Examples of Fc mutations that enhance binding to FcRn are Met252Tyr, Ser254Thr, Thr256Glu (M252Y, S254T, T256E, respectively) (Dall'Acqua et al. (2006) J. BIOL. CHEM. 281(33): 23514-23524), or Met428Leu and Asn434Ser (M428L, N434S) (Zalevsky et al. (2010) NATURE BIOTECH. 28(2): 157-159). Residue changes known to alter affinity for FcRn also fall within the gp68 and/or gE/gI binding footprint. As such, these changes may also lose binding to viral Fc receptors. For example, antibodies with the YTE changes (M252Y/S254T/T256E) lose all binding to HSV-1 gE/gI but not HCMV gp68 (see Example 7). Residue S254 forms two hydrogen bonds with ILE 226 on gE while residues T256 forms close interactions with A319 and A320. The DHS (L309D/Q311H/N434S) and LS (M428L/N434S) Fc variants may also lose binding to gp68 and gE/gI proteins. Within DHS, N434 forms close interactions with A230 on gE while within LS, N434 forms close interactions with A230. All residue numbers are according to EU numbering (Kabat, E. A., et al., supra).

In some aspects, the human IgG constant region is modified to alter antibody-dependent cellular cytotoxicity (ADCC) and/or complement-dependent cytotoxicity (CDC), e.g., the amino acid modifications described in Natsume et al. (2008) CANCER RES. 68(10): 3863-72; Idusogie et al. (2001) J. IMMUNOL. 166(4): 2571-5; Moore et al. (2010) MABS 2(2): 181-189; Lazar et al. (2006) PROC. NATL. ACAD. SCI. USA 103(11): 4005-4010, Shields et al. (2001) J. BIOL. CHEM. 276(9): 6591-6604; Stavenhagen et al. (2007) CANCER RES. 67(18): 8882-8890; Stavenhagen et al. (2008) ADVAN. ENZYME REGUL. 48: 152-164; Alegre et al. (1992) J. IMMUNOL. 148: 3461-3468. In some aspects, the human IgG constant region is modified to alter antibody-dependent phagocytosis (ADCP). For example, Fc variants disclosed herein can be combined with residue changes known to increase Fc binding to classical host Fc receptors CD16a and CD32a and thereby increase ADCC and ADCP functions, respectively. Combining these residue changes is expected to increase the therapeutic window. For example, one or more of the SDALIE changes (G236A, S239D, A330L, 1332E) can be used in combination with the Fc variants disclosed herein.

In some aspects, the human IgG constant region is modified to induce heterodimerization. For example, a heavy chain having an amino acid modification within the CH3 domain at Thr366, e.g., a substitution with a more bulky amino acid, e.g., Tyr (T366W), is able to preferentially pair with a second heavy chain having a CH3 domain having amino acid modifications to less bulky amino acids at positions Thr366, Leu368, and Tyr407, e.g., Ser, Ala and Val, respectively (T366S/L368A/Y407V). Heterodimerization via CH3 modifications can be further stabilized by the introduction of a disulfide bond, for example by changing Ser354 to Cys (S354C) and Y349 to Cys (Y349C) on opposite CH3 domains (see, Carter (2001) J. IMMUNOL. METHODS 248: 7-15).

In some aspects, the constant region of the light chain of the antibody is a human kappa constant region, e.g., a human kappa constant region having the amino acid sequence:

(SEQ ID NO: 8)
TVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVT
KSFNRGEC,

In some aspects, the constant region of the light chain of the antibody is a human kappa constant region, e.g., a human kappa constant region having the amino acid sequence:

(SEQ ID NO: 9)
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSENRGEC

In some aspects, the constant region of the light chain of the antibody is a human lambda constant region, e.g., a human lambda constant region having the amino acid sequence:

(SEQ ID NO: 10)
GQPKANPTVTLFPPSSEELQANKATLVCLISDFYPGAVTVAWKADGSPV
KAGVETTKPSKQSNNKYAASSYLSLTPEQWKSHRSYSCQVTHEGSTVEK
TVAPTEC

A summary of exemplary Fc variants is provided in the following table:

| Fc variant | Residue | | |
|---|---|---|---|
| name | changes | Impact | Reference |
| GASDALIE | G236A, S239D, | Enhance binding | Lazar et al, PNAS |
| | A330L, I332E | to classical Fc | 2006 (PMID |
| | | receptors and | 16537476), |
| | | promote ADCC | Ahmed et al J |
| | | and ADCP | Struct Bio 2016 |
| | | | PMID: 26850169 |
| LS | M428L, N434S | Extend serum | |
| | | half-life through | |
| | | increase FcRn | |
| | | binding at pH 6.0 | |
| | | with low binding | |
| | | at pH 7.4 | |
| DHS | L309D, Q311H, | Extend serum | Lee et al, Nat |
| | N434S | half-life through | Commun 2019 |
| | | increase FcRn | (PMID 31695028) |
| | | binding at pH 6.0 | |
| | | with low binding | |
| | | at pH 7.4 | |
| YTE | M252Y, S254T, | Extend serum | Dall'Aqua et al, |
| | T256E | half-life through | JBC 2006 |
| | | increase FcRn | (PMID 16793771) |
| | | binding at pH 6.0 | |
| | | with low binding | |
| | | at pH 7.4; loses | |
| | | binding to HSV-1 | |
| | | gE/gI | |
| G5 | R255Q, S337F | | This work |
| G8 | S337F | | |
| R47 | H268L, E294K, | | This work |
| | Q311L, K334E | | |
| G2 | R255Q, H268L, | | This work |
| | E294K, Q311L, | | |
| | K334E | | |
| G2B | R255Q, H268L, | | This work |
| | Q311L, K334E | | |
| G2D | R255Q, H268L, | | This work |
| | E294K, Q311L | | |
| G2E | R255Q, H268L, | | This work |
| | Q311L, K334E, | | |
| | S337F | | |

Also provided herein are monoclonal antibodies that bind to HCMV gp34, where the antibodies have clone-paired CDRs from the heavy and light chains as illustrated in Tables 4 and 5 as well as clone-paired variable regions as illustrated in Table 6. Such antibodies may be produced using methods described herein.

TABLE 4
CDRs of heavy and light chain variable sequences of the antibodies as
predicted by Kabat.

| | | CDR1 | CDR2 | CDR3 |
|---|---|---|---|---|
| Clone | Chain | (SEQ ID NO:) | (SEQ ID NO:) | (SEQ ID NO:) |
| 34-2 | Heavy | FNIKDTYMH | RIDPASDDTKYDPKF | DGYYAY |
| | | (SEQ ID NO: 32) | QG | (SEQ ID NO: 34) |
| | | | (SEQ ID NO: 33) | |
| | Light | RASESVDSYGN | RASNLES | QQSYEVPRT |
| | | SFMH | (SEQ ID NO: 36) | (SEQ ID NO: 37) |
| | | (SEQ ID NO: 35) | | |
| 34-4 | Heavy | YTFTAYWMH | AIYPGNSDAGYNQK | WGNNSF |
| | | (SEQ ID NO: 38) | FKG | (SEQ ID NO: 40) |
| | | | (SEQ ID NO: 39) | |
| | Light | KASQDINSYLT | RANRLVD | LQYDEFPPT |
| | | (SEQ ID NO: 41) | (SEQ ID NO: 42) | (SEQ ID NO: 43) |
| 34-5 | Heavy | YAFTNYLIE | VINPGSGGTNYNEK | QVGRLAY |
| | | (SEQ ID NO: 44) | FKG | (SEQ ID NO: 46) |
| | | | (SEQ ID NO: 45) | |
| | Light | KSSRSLLDSDG | LVSKLDS | WQGTHFPPT |
| | | RTYLN | (SEQ ID NO: 48) | (SEQ ID NO: 49) |
| | | (SEQ ID NO: 47) | | |

TABLE 5
CDRs of heavy and light chain variable sequences of the antibodies as
predicted by IMGT/DomainGapAlign (Ehrenmann et al., 2010; Ehrenmann &
Lefranc, 2011).

| | | CDR1 | CDR2 | CDR3 |
|---|---|---|---|---|
| Clone | Chain | (SEQ ID NO:) | (SEQ ID NO:) | (SEQ ID NO:) |
| 34-2 | Heavy | GFNIKDTY | IDPASDDT | SKDGYYAY |
| | | (SEQ ID NO: 50) | (SEQ ID NO: 51) | (SEQ ID NO: 52) |
| | Light | ESVDSYGNSF | RAS | QQSYEVPRT |
| | | (SEQ ID NO: 53) | (SEQ ID NO: 54) | (SEQ ID NO: 37) |
| 34-4 | Heavy | GYTFTAYW | IYPGNSDA | TRWGNNSF |
| | | (SEQ ID NO: 55) | (SEQ ID NO: 56) | (SEQ ID NO: 57) |
| | Light | QDINSY | RAN | LQYDEFPPT |
| | | (SEQ ID NO: 58) | (SEQ ID NO: 59) | (SEQ ID NO: 43) |
| 34-5 | Heavy | GYAFTNYL | INPGSGGT | VLQVGRLAY |
| | | (SEQ ID NO: 60) | (SEQ ID NO: 61) | (SEQ ID NO: 62) |
| | Light | RSLLDSDGRTY | LVS | WQGTHFPPT |
| | | (SEQ ID NO: 63) | (SEQ ID NO: 64) | (SEQ ID NO: 65) |

TABLE 6
Amino acid sequences of the antibody variable regions.

| Clone | Chain | Variable Sequence | SEQ ID NO: |
|---|---|---|---|
| 34-2 | Heavy | QVQLQQSGTELVKPGASVKLSCTASGFNIKDTYMHWVKQR | 66 |
| | | PEQGLEWIGRIDPASDDTKYDPKFQGKAAITADTSSNTAY | |
| | | LQLSSLTSEDTAVYYCSKDGYYAYWGQGTLVTVSA | |
| | Light | DIVMTQSPASLAVSLGQRATISCRASESVDSYGNSFMHWY | 67 |
| | | QQKPGQPPKLLIYRASNLESGIPARFSGSGSRTDFTLTIN | |
| | | PVEADDVATYYCQQSYEVPRTFGGGTKLEIKR | |
| 34-4 | Heavy | QVQLQQSGTVLARPGASVKMSCKASGYTFTAYWMHWVKQR | 68 |
| | | PGQGLEWIGAIYPGNSDAGYNQKFKGKAKLTAVTSTSTAY | |
| | | MELSSLANEDSAVYYCTRWGNNSFWGQGTTLTVSS | |
| | Light | DIQMTQSPSSMYASLGERVTITCKASQDINSYLTWFQQKP | 69 |
| | | GKSPKTLIYRANRLVDGVPSRFSGSGSGQDYSLTISTLEY | |
| | | EDMGIYYCLQYDEFPPTFGAGTKLELKR | |
| 34-5 | Heavy | QVQLQQSGAELVRPGTSVKVSCKTSGYAFTNYLIEWVKQR | 70 |
| | | PGQGLEWIGVINPGSGGTNYNEKFKGKATLTADKSSSTAY | |
| | | MQLSSLTSDDSAVYFCVLQVGRLAYWGQGTLVTVSA | |
| | Light | DIQMTQSPLTLSVTIGQPASISCKSSRSLLDSDGRTYLNW | 71 |
| | | LLQRPGQSPKRLIYLVSKLDSGVPDRFTGSGSGTDFTLKI | |
| | | SRVEAEDLGVYYCWQGTHFPPTFGAGTKLELKR | |

The antibodies and antibody fragments of the present invention have several applications, include the diagnosis and treatment of diseases. As such, antibodies or antibody fragments may be linked diagnostic or therapeutic agents or used without additional agents being attached thereto. The antibodies or antibody fragments may be mutated or modified, as discussed further below. Methods for preparing and characterizing antibodies are well known in the art (see, e.g., Antibodies: A Laboratory Manual, Cold Spring Harbor Laboratory, 1988; U.S. Pat. No. 4,196,265).

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also fragments thereof (such as Fab, Fab′, F(ab′)₂, Fv, Fd, Fd′, single chain antibody (ScFv), diabody, linear antibody), mutants thereof, naturally occurring variants, fusion proteins comprising an antibody portion with an antigen recognition site of the required specificity, humanized antibodies, chimeric antibodies, and any other modified configuration of the immunoglobulin molecule that comprises an antigen recognition site of the required specificity.

An “isolated antibody” is an antibody that has been separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. In particular instances, the antibody is purified: (1) to greater than 95% by weight of antibody as determined by the Lowry method, and most particularly more than 99% by weight; or (2) to homogeneity by SDS-PAGE under reducing or non-reducing conditions using Coomassie blue or silver stain. Isolated antibody includes the antibody in situ within recombinant cells since at least one component of the antibody's natural environment will not be present. Ordinarily, however, an isolated antibody will be prepared by at least one purification step.

The basic four-chain antibody unit is a heterotetrameric glycoprotein composed of two identical light (L) chains and two identical heavy (H) chains. The term “heavy chain” as used herein refers to the larger immunoglobulin subunit which associates, through its amino terminal region, with the immunoglobulin light chain. The heavy chain comprises a variable region (V_{H}) and a constant region (CH). The constant region further comprises the C_{H}1, hinge, C_{H}2, and C_{H}3 domains. In the case of IgE, IgM, and IgY, the heavy chain comprises a C_{H}4 domain but does not have a hinge domain. Those skilled in the art will appreciate that heavy chains are classified as gamma, mu, alpha, delta, or epsilon (γ, μ, α, δ, ε), with some subclasses among them (e.g., γ1-γ4, α1-α2). It is the nature of this chain that determines the “class” of the antibody as IgG, IgM, IgA IgD, or IgE, respectively. The immunoglobulin subclasses (isotypes), e.g., IgG1, IgG2, IgG3, IgG4, IgA1, etc. are well characterized and are known to confer functional specialization.

The term “light chain” as used herein refers to the smaller immunoglobulin subunit which associates with the amino terminal region of a heavy chain. As with a heavy chain, a light chain comprises a variable region (V_{L}) and a constant region (C_{L}). Light chains are classified as either kappa or lambda (κ, λ) based on the amino acid sequences of their constant domains (C_{L}). A pair of these can associate with a pair of any of the various heavy chains to form an immunoglobulin molecule. Also encompassed in the meaning of light chain are light chains with a lambda variable region (V-lambda) linked to a kappa constant region (C-kappa) or a kappa variable region (V-kappa) linked to a lambda constant region (C-lambda).

In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable region (V_{H}) followed by three constant domains (C_{H}) for each of the alpha and gamma chains and four C_{H} domains for mu and isotypes. Each L chain has at the N-terminus, a variable region (V_{L}) followed by a constant domain (C_{L}) at its other end. The V_{L} is aligned with the V_{H} and the C_{L} is aligned with the first constant domain of the heavy chain (C_{H}1). Particular amino acid residues are believed to form an interface between the light chain and heavy chain variable regions. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71, and Chapter 6.

A “variable region” of an antibody refers to the variable region of the antibody light chain or the variable region of the antibody heavy chain, either alone or in combination. The term “variable” refers to the fact that certain segments of the variable regions differ extensively in sequence among antibodies. The variable regions of both the light (V_{L}) and heavy (V_{H}) chain portions mediate antigen binding and define the specificity of a particular antibody for its particular antigen. However, the variability is not evenly distributed across the entirety of the variable regions. Instead, the variable regions consist of relatively invariant stretches called framework regions (FRs) separated by shorter regions of extreme variability called complementarity determining regions (CDRs) or hypervariable regions. The variable regions of native heavy and light chains each comprise four FRs, largely adopting a beta-sheet configuration, connected by three CDRs, which form loops connecting, and in some cases forming part of, the beta-sheet structure. The CDRs complement an antigen's shape and determine the antibody's affinity and specificity for the antigen. There are six CDRs in both V_{L} and V_{H}. The CDRs in each chain are held together in close proximity by the FRs and, with the CDRs from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)).

The term “hypervariable region” when used herein refers to the amino acid residues of an antibody that are responsible for antigen binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35 (H1), 50-65 (H2) and 95-102 (H3) in the V_{H} when numbered in accordance with the Kabat numbering system; Kabat et al., Sequences of Proteins of Immunological Interest, 5th Ed. Public Health Service, National Institutes of Health, Bethesda, Md. (1991)); and/or those residues from a “hypervariable loop” (e.g., residues 24-34 (L1), 50-56 (L2) and 89-97 (L3) in the V_{L}, and 26-32 (H1), 52-56 (H2) and 95-101 (H3) in the V_{H} when numbered in accordance with the Chothia numbering system; Chothia and Lesk, J. Mol. Biol. 196:901-917 (1987)); and/or those residues from a “hypervariable loop”/CDR (e.g., residues 27-38 (L1), 56-65 (L2) and 105-120 (L3) in the V_{L}, and 27-38 (H1), 56-65 (H2) and 105-120 (H3) in the V_{H} when numbered in accordance with the IMGT numbering system; Lefranc, M. P. et al. Nucl. Acids Res. 27:209-212 (1999), Ruiz, M. et al. Nucl. Acids Res. 28:219-221 (2000)). Optionally the antibody has symmetrical insertions at one or more of the following points 28, 36 (L1), 63, 74-75 (L2) and 123 (L3) in the V_{L}, and 28, 36 (H1), 63, 74-75 (H2) and 123 (H3) in the V_{H} when numbered in accordance with AHo; Honneger, A. and Plunkthun, A. J. Mol. Biol. 309:657-670 (2001)). As used herein, a CDR may refer to CDRs defined by any of these numbering approaches or by a combination of approaches or by other desirable approaches. In addition, a new definition of highly conserved core, boundary and hyper-variable regions can be used.

A “constant region” of an antibody refers to the constant region of the antibody light chain or the constant region of the antibody heavy chain, either alone or in combination. The constant regions of the light chain (CL) and the heavy chain (C_{H}1, C_{H}2 or C_{H}3, or C_{H}4 in the case of IgM and IgE) confer important biological properties such as secretion, transplacental mobility, Fc receptor binding, complement binding, and the like. By convention the numbering of the constant region domains increases as they become more distal from the antigen binding site or amino-terminus of the antibody. The constant regions are not involved directly in binding an antibody to an antigen, but exhibit various effector functions, such as participation of the antibody in antibody dependent cellular cytotoxicity (ADCC), antibody-dependent cellular phagocytosis (ADCP), antibody-dependent neutrophil phagocytosis (ADNP), and antibody-dependent complement deposition (ADCD).

The antibody may be an antibody fragment. “Antibody fragments” comprise only a portion of an intact antibody, generally including an antigen binding site of the intact antibody and thus retaining the ability to bind antigen. Examples of antibody fragments encompassed by the present definition include: (i) the Fab fragment, having V_{L}, C_{L}, V_{H} and C_{H1} domains; (ii) the Fab′ fragment, which is a Fab fragment having one or more cysteine residues at the C-terminus of the C_{H1} domain; (iii) the Fd fragment having V_{H} and C_{H}1 domains; (iv) the Fd′ fragment having V_{H} and C_{H}1 domains and one or more cysteine residues at the C-terminus of the C_{H}1 domain; (v) the Fv fragment having the V_{L} and V_{H} domains of a single antibody; (vi) the dAb fragment which consists of a V_{H} domain; (vii) isolated CDR regions; (viii) F(ab′)₂ fragments, a bivalent fragment including two Fab′ fragments linked by a disulfide bridge at the hinge region; (ix) single chain antibody molecules (e.g. single chain Fv; scFv); (x) “diabodies” with two antigen binding sites, comprising a heavy chain variable domain (V_{H}) connected to a light chain variable domain (V_{L}) in the same polypeptide chain; (xi) “linear antibodies” comprising a pair of tandem Fd segments (V_{H}-C_{H}1-V_{H}-C_{H}1) which, together with complementary light chain polypeptides, form a pair of antigen binding regions.

The antibody may be a chimeric antibody. “Chimeric antibodies” refers to those antibodies wherein one portion of each of the amino acid sequences of heavy and light chains is homologous to corresponding sequences in antibodies derived from a particular species or belonging to a particular class, while the remaining segment of the chains is homologous to corresponding sequences in another. For example, a chimeric antibody may be an antibody comprising antigen binding sequences from a non-human donor grafted to a heterologous non-human, human, or humanized sequence (e.g., framework and/or constant domain sequences). Typically, in these chimeric antibodies, the variable region of both light and heavy chains mimics the variable regions of antibodies derived from one species of mammals, while the constant portions are homologous to the sequences in antibodies derived from another. For example, methods have been developed to replace light and heavy chain constant domains of a monoclonal antibody with analogous domains of human origin, leaving the variable regions of the foreign antibody intact. Alternatively, “fully human” monoclonal antibodies are produced in mice transgenic for human immunoglobulin genes. Methods have also been developed to convert variable domains of monoclonal antibodies to more human form by recombinantly constructing antibody variable domains having both rodent, for example, mouse, and human amino acid sequences. In “humanized” monoclonal antibodies, only the hypervariable CDR is derived from mouse monoclonal antibodies, and the framework and constant regions are derived from human amino acid sequences (see U.S. Pat. Nos. 5,091,513 and 6,881,557, incorporated herein by reference). It is thought that replacing amino acid sequences in the antibody that are characteristic of rodents with amino acid sequences found in the corresponding position of human antibodies will reduce the likelihood of adverse immune reaction during therapeutic use. A hybridoma or other cell producing an antibody may also be subject to genetic mutation or other changes, which may or may not alter the binding specificity of antibodies produced by the hybridoma.

### B. Monoclonal Antibodies

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, monoclonal antibodies are advantageous in that they may be synthesized uncontaminated by other antibodies. The modifier “monoclonal” is not to be construed as requiring production of the antibody by any particular method. For example, the monoclonal antibodies useful in the present disclosure may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567) after single cell sorting of an antigen specific B cell, an antigen specific plasmablast responding to an infection or immunization, or capture of linked heavy and light chains from single cells in a bulk sorted antigen specific collection. The monoclonal antibodies may also be isolated from phage antibody libraries using the techniques described in Clackson et al., Nature, 352:624-628 (1991) and Marks et al., J. Mol. Biol., 222:581-597 (1991), for example.

Methods for producing monoclonal antibodies of various types, including humanized, chimeric, and fully human, are well known in the art and highly predictable. For example, the following U.S. patents and patent applications provide enabling descriptions of such methods: U.S. Patent Application Nos. 2004/0126828 and 2002/0172677; and U.S. Pat. Nos. 3,817,837; 3,850,752; 3,939,350; 3,996,345; 4,196,265; 4,275,149; 4,277,437; 4,366,241; 4,469,797; 4,472,509; 4,606,855; 4,703,003; 4,742,159; 4,767,720; 4,816,567; 4,867,973; 4,938,948; 4,946,778; 5,021,236; 5,164,296; 5,196,066; 5,223,409; 5,403,484; 5,420,253; 5,565,332; 5,571,698; 5,627,052; 5,656,434; 5,770,376; 5,789,208; 5,821,337; 5,844,091; 5,858,657; 5,861,155; 5,871,907; 5,969,108; 6,054,297; 6,165,464; 6,365,157; 6,406,867; 6,709,659; 6,709,873; 6,753,407; 6,814,965; 6,849,259; 6,861,572; 6,875,434; and 6,891,024, each incorporated herein by reference.

### C. Bispecific and Multispecific Antibodies

Antibodies may be bispecific or multispecific. “Bispecific antibodies” are antibodies that have binding specificities for at least two different epitopes. Exemplary bispecific antibodies may bind to two different epitopes of a single antigen. Other such antibodies may combine a first antigen binding site with a binding site for a second antigen. Alternatively, an antigen-specific arm may be combined with an arm that binds to a triggering molecule on a leukocyte, such as a T-cell receptor molecule (e.g., CD3), or Fc receptors for IgG (FcγR), such as FcγRI (CD64), FcγRII (CD32) and Fc gamma RIII (CD16), so as to focus and localize cellular defense mechanisms to the infected cell. Bispecific antibodies may also be used to localize cytotoxic agents to infected cells. These antibodies possess an antigen-binding arm and an arm that binds the cytotoxic agent (e.g., saporin, anti-interferon-α, vinca alkaloid, ricin A chain, methotrexate or radioactive isotope hapten). Bispecific antibodies can be prepared as full-length antibodies or antibody fragments (e.g., F(ab′)₂ bispecific antibodies). Taki et al. (2015) describes a bispecific anti-HSP70/anti-CD3 antibody.

Methods for making bispecific antibodies are known in the art. Traditional production of full-length bispecific antibodies is based on the co-expression of two immunoglobulin heavy chain-light chain pairs, where the two chains have different specificities. Because of the random assortment of immunoglobulin heavy and light chains, these hybridomas (quadromas) produce a potential mixture of ten different antibody molecules, of which only one has the correct bispecific structure. Purification of the correct molecule, which is usually done by affinity chromatography steps, is rather cumbersome, and the product yields are low.

According to a different approach, antibody variable regions with the desired binding specificities (antibody-antigen combining sites) are fused to immunoglobulin constant domain sequences. Preferably, the fusion is with an Ig heavy chain constant domain, comprising at least part of the hinge, C_{H2}, and C_{H3} regions. It is preferred to have the first heavy-chain constant region (C_{H1}) containing the site necessary for light chain bonding, present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the three polypeptide fragments when unequal ratios of the three polypeptide chains used in the construction provide the optimum yield of the desired bispecific antibody. It is, however, possible to insert the coding sequences for two or all three polypeptide chains into a single expression vector when the expression of at least two polypeptide chains in equal ratios results in high yields or when the ratios have no significant effect on the yield of the desired chain combination.

The bispecific antibodies may be composed of a hybrid immunoglobulin heavy chain with a first binding specificity in one arm, and a hybrid immunoglobulin heavy chain-light chain pair (providing a second binding specificity) in the other arm. This asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach described in U.S. Pat. No. 5,731,168, the interface between a pair of antibody molecules can be engineered to maximize the percentage of heterodimers that are recovered from recombinant cell culture. The preferred interface comprises at least a part of the C_{H3} domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)₂ fragments. These fragments are reduced in the presence of the dithiol complexing agent, sodium arsenite, to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Techniques exist that facilitate the direct recovery of Fab′-SH fragments from E. coli, which can be chemically coupled to form bispecific antibodies. Shalaby et al., J. Exp. Med., 175: 217-225 (1992) describe the production of a humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described (Merchant et al., Nat. Biotechnol. 16, 677-681 (1998)). For example, bispecific antibodies have been produced using leucine zippers (Kostelny et al., J. Immunol., 148(5):1547-1553, 1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a V_{H} connected to a V_{L} by a linker that is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

A bispecific or multispecific antibody may be formed as a DOCK-AND-LOCK™ (DNL™) complex (see, e.g., U.S. Pat. Nos. 7,521,056; 7,527,787; 7,534,866; 7,550,143 and 7,666,400). Generally, the technique takes advantage of the specific and high-affinity binding interactions that occur between a dimerization and docking domain (DDD) sequence of the regulatory (R) subunits of cAMP-dependent protein kinase (PKA) and an anchor domain (AD) sequence derived from any of a variety of AKAP proteins (Baillie et al., FEBS Letters. 2005; 579: 3264; Wong and Scott, Nat. Rev. Mol. Cell Biol. 2004; 5: 959). The DDD and AD peptides may be attached to any protein, peptide or other molecule. Because the DDD sequences spontaneously dimerize and bind to the AD sequence, the technique allows the formation of complexes between any selected molecules that may be attached to DDD or AD sequences.

Antibodies with more than two valencies are contemplated. For example, trispecific antibodies can be prepared (Tutt et al., J. Immunol. 147: 60, 1991; Xu et al., Science, 358(6359):85-90, 2017). The antibodies may also involve sequences or moieties that permit dimerization or multimerization of the receptors. Such sequences include those derived from IgA, which permit formation of multimers in conjunction with the J-chain. Another multimerization domain is the Gal4 dimerization domain.

A multivalent antibody may be internalized (and/or catabolized) faster than a bivalent antibody by a cell expressing an antigen to which the antibody binds. The antibodies of the present disclosure can be multivalent antibodies with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. Multivalent antibodies may comprise (or consist of) three to about eight, for example four, antigen binding sites. The multivalent antibody comprises at least one polypeptide chain (and preferably two polypeptide chains), wherein the polypeptide chain(s) comprise two or more variable regions. For instance, the polypeptide chain(s) may comprise VD1-(X1).sub.n-VD2-(X2)˜-Fc, wherein VD1 is a first variable region, VD2 is a second variable region, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein may further comprise at least two (and preferably four) light chain variable region polypeptides. The multivalent antibody herein may, for instance, comprise from about two to about eight light chain variable region polypeptides. The light chain variable region polypeptides contemplated here comprise a light chain variable region and, optionally, further comprise a C_{L} domain.

Charge modifications are particularly useful in the context of a multispecific antibody, where amino acid substitutions in Fab molecules result in reducing the mispairing of light chains with non-matching heavy chains (Bence-Jones-type side products), which can occur in the production of Fab-based bi-/multispecific antigen binding molecules with a VH/VL exchange in one (or more, in case of molecules comprising more than two antigen-binding Fab molecules) of their binding arms (see also PCT publication no. WO 2015/150447, particularly the examples therein, incorporated herein by reference in its entirety).

### D. Antibody Conjugates

Antibodies of the present disclosure may be linked to at least one agent to form an antibody conjugate. The conjugate can be, for example, an antibody conjugated to another proteinaceous, carbohydrate, lipid, or mixed moiety molecule(s). Such antibody conjugates include, but are not limited to, modifications that include linking the antibody to one or more polymers. For example, an antibody may be linked to one or more water-soluble polymers. Linkage to a water-soluble polymer reduces the likelihood that the antibody will precipitate in an aqueous environment, such as a physiological environment. One skilled in the art can select a suitable water-soluble polymer based on considerations including, but not limited to, whether the polymer/antibody conjugate will be used in the treatment of a patient and, if so, the pharmacological profile of the antibody (e.g., half-life, dosage, activity, antigenicity, and/or other factors).

In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to antibodies include toxins, anti-tumor agents, therapeutic enzymes, radionuclides, antiviral agents, chelating agents, cytokines, growth factors, and oligo- or polynucleotides. By contrast, a reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, photoaffinity molecules, colored particles or ligands, an enzyme (e.g., that catalyzes a colorimetric or fluorometric reaction), a substrate, a solid matrix, such as biotin. An antibody may comprise one, two, or more of any of these labels.

Antibody conjugates may be used to deliver cytotoxic agents to target cells. Cytotoxic agents of this type may improve antibody-mediated cytotoxicity, and include such moieties as cytokines that directly or indirectly stimulate cell death, radioisotopes, chemotherapeutic drugs (including prodrugs), bacterial toxins (e.g., pseudomonas exotoxin, diphtheria toxin, etc.), plant toxins (e.g., ricin, gelonin, etc.), chemical conjugates (e.g., maytansinoid toxins, auristatins, α-amanitin, anthracyclines, calechaemicin, etc.), radioconjugates, enzyme conjugates (e.g., RNase conjugates, granzyme antibody-directed enzyme/prodrug therapy), and the like.

Antibody conjugates are also used as diagnostic agents. Antibody diagnostics generally fall within two classes, those for use in in vitro diagnostics, such as in a variety of immunoassays, and those for use in vivo diagnostic protocols, generally known as “antibody-directed imaging.” Many appropriate imaging agents are known in the art, as are methods for their attachment to antibodies (see, for e.g., U.S. Pat. Nos. 5,021,236, 4,938,948, and 4,472,509). The imaging moieties used can be paramagnetic ions, radioactive isotopes, fluorochromes, NMR-detectable substances, and X-ray imaging agents.

The paramagnetic ions contemplated for use as conjugates include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and bismuth (III).

The radioactive isotopes contemplated for use as conjugated include astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and/or yttrium⁹⁰. ¹²⁵I is often being preferred. Technicium^{99m} and/or indium¹¹¹ are also often preferred due to their low energy and suitability for long range detection. Radioactively labeled monoclonal antibodies of the present disclosure may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the disclosure may be labeled with technetium^{99m} by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

The fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Additional types of antibodies contemplated in the present disclosure are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and avidin and streptavidin compounds.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

Another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light. In particular, 2- and 8-azido analogues of purine nucleotides have been used as site-directed photoprobes to identify nucleotide binding proteins in crude cell extracts. The 2- and 8-azido nucleotides have also been used to map nucleotide binding domains of purified proteins and may be used as antibody binding agents.

Derivatization of immunoglobulins by selectively introducing sulfhydryl groups in the Fc region of an immunoglobulin, using reaction conditions that do not alter the antibody combining site are also contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature. This approach has been reported to produce diagnostically and therapeutically promising antibodies which are currently in clinical evaluation.

### E. Antibody Drug Conjugates

Antibody drug conjugates, or ADCs, are a new class of highly potent biopharmaceutical drugs designed as a targeted therapy for the treatment of people with disease. ADCs are complex molecules composed of an antibody (a whole mAb or an antibody fragment, such as a scFv) linked, via a stable chemical linker with labile bonds, to a biological active cytotoxic/anti-viral payload or drug. Antibody drug conjugates are examples of bioconjugates and immunoconjugates.

By combining the unique targeting capabilities of monoclonal antibodies with the cancer-killing ability of cytotoxic drugs, antibody-drug conjugates allow sensitive discrimination between healthy and diseased tissue. This means that, in contrast to traditional systemic approaches, antibody-drug conjugates target and attack the diseased cell so that healthy cells are less severely affected.

In the development ADC-based anti-tumor therapies, an anticancer drug (e.g., a cell toxin or cytotoxin) is coupled to an antibody that specifically targets a certain cell marker (e.g., a protein that, ideally, is only to be found in or on diseased cells). Antibodies track these proteins down in the body and attach themselves to the surface of the diseased cells. The biochemical reaction between the antibody and the target protein (antigen) triggers a signal in the targeted cell, which then absorbs or internalizes the antibody together with the cytotoxin. After the ADC is internalized, the cytotoxic drug is released and kills the cell or impairs cellular replication. Due to this targeting, ideally the drug has lower side effects and gives a wider therapeutic window than other agents.

A stable link between the antibody and cytotoxic agent is a crucial aspect of an ADC. Linkers are based on chemical motifs including disulfides, hydrazones or peptides (cleavable), or thioethers (noncleavable) and control the distribution and delivery of the cytotoxic agent to the target cell. Cleavable and non-cleavable types of linkers have been proven to be safe in preclinical and clinical trials. Brentuximab vedotin includes an enzyme-sensitive cleavable linker that delivers the potent and highly toxic antimicrotubule agent Monomethyl auristatin E or MMAE, a synthetic antineoplastic agent, to human specific CD30-positive malignant cells. Because of its high toxicity MMAE, which inhibits cell division by blocking the polymerization of tubulin, cannot be used as a single-agent chemotherapeutic drug. However, the combination of MMAE linked to an anti-CD30 monoclonal antibody (cAC10, a cell membrane protein of the tumor necrosis factor or TNF receptor) proved to be stable in extracellular fluid, cleavable by cathepsin and safe for therapy. Trastuzumab emtansine, the other approved ADC, is a combination of the microtubule-formation inhibitor mertansine (DM-1), a derivative of the Maytansine, and antibody trastuzumab (Herceptin®/Genentech/Roche) attached by a stable, non-cleavable linker.

The availability of better and more stable linkers has changed the function of the chemical bond. The type of linker, cleavable or noncleavable, lends specific properties to the cytotoxic (e.g., anti-cancer) drug. For example, a non-cleavable linker keeps the drug within the cell. As a result, the entire antibody, linker, and cytotoxic agent enter the targeted cell where the antibody is degraded to the level of amino acids. The resulting complex—amino acid, linker and cytotoxic agent—now becomes the active drug. In contrast, cleavable linkers are catalyzed by enzymes in the host cell, thereby releasing the cytotoxic agent.

Another type of cleavable linker adds an extra molecule between the cytotoxic drug and the cleavage site. This linker technology allows researchers to create ADCs with more flexibility without worrying about changing cleavage kinetics. Researchers are also developing a new method of peptide cleavage based on Edman degradation. Future direction in the development of ADCs also include the development of site-specific conjugation (TDCs) to further improve stability and therapeutic index and α-emitting immunoconjugates and antibody-conjugated nanoparticles.

### F. Production and Purification of Antibodies

Recombinant full-length IgG antibodies can be generated by subcloning heavy and light chain Fv DNAs from the cloning vector into an IgG plasmid vector, transfected into 293 (e.g., Freestyle) cells or CHO cells, and antibodies can be collected and purified from the 293 or CHO cell supernatant. Other appropriate host cells systems include bacteria, such as E. coli, insect cells (S2, Sf9, Sf29, High Five), plant cells (e.g., tobacco, with or without engineering for human-like glycans), algae, or in a variety of non-human transgenic contexts, such as mice, rats, goats or cows.

Expression of nucleic acids encoding antibodies, both for the purpose of subsequent antibody purification, and for immunization of a host, is also contemplated. Antibody coding sequences can be RNA, such as native RNA or modified RNA. Modified RNA contemplates certain chemical modifications that confer increased stability and low immunogenicity to mRNAs, thereby facilitating expression of therapeutically important proteins. For instance, N1-methyl-pseudouridine (N1mΨ) outperforms several other nucleoside modifications and their combinations in terms of translation capacity. In addition to turning off the immune/eIF2α phosphorylation-dependent inhibition of translation, incorporated N1mΨ nucleotides dramatically alter the dynamics of the translation process by increasing ribosome pausing and density on the mRNA. Increased ribosome loading of modified mRNAs renders them more permissive for initiation by favoring either ribosome recycling on the same mRNA or de novo ribosome recruitment. Such modifications could be used to enhance antibody expression in vivo following inoculation with RNA. The RNA, whether native or modified, may be delivered as naked RNA or in a delivery vehicle, such as a lipid nanoparticle.

Alternatively, DNA encoding the antibody may be employed for the same purposes. The DNA is included in an expression cassette comprising a promoter active in the host cell for which it is designed. The expression cassette is advantageously included in a replicable vector, such as a conventional plasmid or minivector. Vectors include viral vectors, such as poxviruses, adenoviruses, herpesviruses, adeno-associated viruses, and lentiviruses are contemplated. Replicons encoding antibody genes such as alphavirus replicons based on VEE virus or Sindbis virus are also contemplated. Delivery of such vectors can be performed by needle through intramuscular, subcutaneous, or intradermal routes, or by transcutaneous electroporation when in vivo expression is desired.

Alternatively, a molecular cloning approach may be used to generate monoclonal antibodies. Single B cells labeled with the antigen of interest can be sorted physically using paramagnetic bead selection or flow cytometric sorting, then RNA can be isolated from the single cells and antibody genes amplified by RT-PCR. Alternatively, antigen-specific bulk sorted populations of cells can be segregated into microvesicles and the matched heavy and light chain variable genes recovered from single cells using physical linkage of heavy and light chain amplicons, or common barcoding of heavy and light chain genes from a vesicle. Matched heavy and light chain genes form single cells also can be obtained from populations of antigen specific B cells by treating cells with cell-penetrating nanoparticles bearing RT-PCR primers and barcodes for marking transcripts with one barcode per cell. The antibody variable genes also can be isolated by RNA extraction of a hybridoma line and the antibody genes obtained by RT-PCR and cloned into an immunoglobulin expression vector. Alternatively, combinatorial immunoglobulin phagemid libraries are prepared from RNA isolated from the cell lines and phagemids expressing appropriate antibodies are selected by panning using viral antigens. The advantages of this approach over conventional hybridoma techniques are that approximately 10⁴ times as many antibodies can be produced and screened in a single round, and that new specificities are generated by H and L chain combination which further increases the chance of finding appropriate antibodies.

Other U.S. patents, each incorporated herein by reference, that teach the production of antibodies useful in the present disclosure include U.S. Pat. No. 5,565,332, which describes the production of chimeric antibodies using a combinatorial approach; U.S. Pat. No. 4,816,567 which describes recombinant immunoglobulin preparations; and U.S. Pat. No. 4,867,973 which describes antibody-therapeutic agent conjugates.

Monoclonal antibodies produced by any means may be purified, if desired, using filtration, centrifugation, and various chromatographic methods, such as FPLC or affinity chromatography. Fragments of the monoclonal antibodies of the disclosure can be obtained from the purified monoclonal antibodies by methods that include digestion with enzymes, such as pepsin or papain, and/or by cleavage of disulfide bonds by chemical reduction. Alternatively, monoclonal antibody fragments encompassed by the present disclosure can be synthesized using an automated peptide synthesizer.

The antibodies of the present disclosure may be purified. The term “purified,” as used herein, is intended to refer to a composition, isolatable from other components, wherein the protein is purified to any degree relative to its naturally obtainable state. A purified protein therefore also refers to a protein, free from the environment in which it may naturally occur. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include, precipitation with ammonium sulfate, PEG, antibodies and the like or by heat denaturation, followed by centrifugation; gel filtration, reverse phase, hydroxyapatite and affinity chromatography; and combinations of such and other techniques.

In purifying an antibody of the present disclosure, it may be desirable to express the polypeptide in a prokaryotic or eukaryotic expression system and extract the protein using denaturing conditions. The polypeptide may be purified from other cellular components using an affinity column, which binds to a tagged portion of the polypeptide. As is generally known in the art, it is believed that the order of conducting the various purification steps may be changed, or that certain steps may be omitted, and still result in a suitable method for the preparation of a substantially purified protein or peptide.

Commonly, complete antibodies are fractionated utilizing agents (i.e., protein A) that bind the Fc portion of the antibody. Alternatively, antigens may be used to simultaneously purify and select appropriate antibodies. Such methods often utilize the selection agent bound to a support, such as a column, filter or bead. The antibodies are bound to a support, contaminants removed (e.g., washed away), and the antibodies released by applying conditions (salt, heat, etc.).

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. Another method for assessing the purity of a fraction is to calculate the specific activity of the fraction, to compare it to the specific activity of the initial extract, and to thus calculate the degree of purity. The actual units used to represent the amount of activity will, of course, be dependent upon the particular assay technique chosen to follow the purification and whether or not the expressed protein or peptide exhibits a detectable activity.

It is known that the migration of a polypeptide can vary, sometimes significantly, with different conditions of SDS/PAGE. It will therefore be appreciated that under differing electrophoresis conditions, the apparent molecular weights of purified or partially purified expression products may vary.

### G. Modification of Antibodies

The sequences of antibodies may be modified for a variety of reasons, such as improved expression, improved cross-reactivity, or diminished off-target binding. Modified antibodies may be made by any technique known to those of skill in the art, including expression through standard molecular biological techniques, or the chemical synthesis of polypeptides.

For example, one may wish to make modifications, such as introducing conservative changes into an antibody molecule. In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

The substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: basic amino acids: arginine (+3.0), lysine (+3.0), and histidine (−0.5); acidic amino acids: aspartate (+3.0±1), glutamate (+3.0±1), asparagine (+0.2), and glutamine (+0.2); hydrophilic, nonionic amino acids: serine (+0.3), asparagine (+0.2), glutamine (+0.2), and threonine (−0.4), sulfur containing amino acids: cysteine (−1.0) and methionine (−1.3); hydrophobic, nonaromatic amino acids: valine (−1.5), leucine (−1.8), isoleucine (−1.8), proline (−0.5±1), alanine (−0.5), and glycine (0); hydrophobic, aromatic amino acids: tryptophan (−3.4), phenylalanine (−2.5), and tyrosine (−2.3).

An amino acid can be substituted for another having a similar hydrophilicity and produce a biologically or immunologically modified protein. In such changes, the substitution of amino acids whose hydrophilicity values are within ±2 is preferred, those that are within ±1 are particularly preferred, and those within ±0.5 are even more particularly preferred.

Amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

The present disclosure also contemplates isotype modification. By modifying the Fc region to have a different isotype, different functionalities can be achieved. For example, changing to IgG₁ can increase antibody dependent cell cytotoxicity, switching to class A can improve tissue distribution, and switching to class M can improve valency.

One can design an Fc region of an antibody with altered effector function, e.g., by modifying C1q binding and/or FcγR binding and thereby changing CDC activity and/or ADCC activity. “Effector functions” are responsible for activating or diminishing a biological activity (e.g., in a subject). Examples of effector functions include, but are not limited to: C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions may require the Fc region to be combined with a binding domain (e.g., an antibody variable domain) and can be assessed using various assays (e.g., Fc binding assays, ADCC assays, CDC assays, etc.).

For example, one can generate a variant Fc region of an antibody with improved C1q binding and improved FcγRIII binding (e.g., having both improved ADCC activity and improved CDC activity). Alternatively, if it is desired that effector function be reduced or ablated, a variant Fc region can be engineered with reduced CDC activity and/or reduced ADCC activity. In other embodiments, only one of these activities may be increased, and, optionally, also the other activity reduced (e.g., to generate an Fc region variant with improved ADCC activity, but reduced CDC activity and vice versa).

An isolated monoclonal antibody, or antigen binding fragment thereof, may contain a substantially homogeneous glycan without sialic acid, galactose, or fucose. The aforementioned substantially homogeneous glycan may be covalently attached to the heavy chain constant region.

A monoclonal antibody may have a novel Fc glycosylation pattern. Glycosylation of an Fc region is typically either N-linked or O-linked. N-linked refers to the attachment of the carbohydrate moiety to the side chain of an asparagine residue. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used. The recognition sequences for enzymatic attachment of the carbohydrate moiety to the asparagine side chain peptide sequences are asparagine-X-serine and asparagine-X-threonine, where X is any amino acid except proline. Thus, the presence of either of these peptide sequences in a polypeptide creates a potential glycosylation site.

The glycosylation pattern may be altered, for example, by deleting one or more glycosylation site(s) found in the polypeptide, and/or adding one or more glycosylation site(s) that are not present in the polypeptide. Addition of glycosylation sites to the Fc region of an antibody is conveniently accomplished by altering the amino acid sequence such that it contains one or more of the above-described tripeptide sequences (for N-linked glycosylation sites). An exemplary glycosylation variant has an amino acid substitution of residue Asn 297 of the heavy chain. The alteration may also be made by the addition of, or substitution by, one or more serine or threonine residues to the sequence of the original polypeptide (for O-linked glycosylation sites). Additionally, a change of Asn 297 to Ala can remove one of the glycosylation sites.

The isolated monoclonal antibody, or antigen binding fragment thereof, may be present in a substantially homogenous composition represented by the GNGN or G1/G2 glycoform, which exhibits increased binding affinity for Fc gamma RI and Fc gamma RIII compared to the same antibody without the substantially homogeneous GNGN glycoform and with G0, G1F, G2F, GNF, GNGNF or GNGNFX containing glycoforms. Fc glycosylation plays a significant role in anti-viral and anti-cancer properties of therapeutic mAbs. Elimination of core fucose dramatically improves the ADCC activity of mAbs mediated by natural killer (NK) cells but appears to have the opposite effect on the ADCC activity of polymorphonuclear cells (PMNs).

The isolated monoclonal antibody, or antigen binding fragment thereof, may be expressed in cells that express beta (1,4)—N-acetylglucosaminyltransferase III (GnT III), such that GnT III adds GlcNAc to the antibody. Methods for producing antibodies in such a fashion are provided in WO/9954342 and WO/03011878. Cell lines can be altered to enhance or reduce or eliminate certain post-translational modifications, such as glycosylation, using genome editing technology such as Clustered Regularly Interspaced Short Palindromic Repeats (CRISPR). For example, CRISPR technology can be used to eliminate genes encoding glycosylating enzymes in 293 or CHO cells used to express monoclonal antibodies.

It is possible to engineer the antibody variable gene sequences obtained from human B cells to enhance their manufacturability and safety. Potential protein sequence liabilities can be identified by searching for sequence motifs associated with sites containing:
1) Unpaired Cys residues,
2) N-linked glycosylation,
3) Asn deamidation,
4) Asp isomerization,
5) SYE truncation,
6) Met oxidation,
7) Trp oxidation,
8) N-terminal glutamate,
9) Integrin binding,
10) CD11c/CD18 binding, or
11) Fragmentation
   Such motifs can be eliminated by altering the synthetic gene comprising the cDNA encoding the antibodies.

Antibodies can be engineered to enhance solubility. For example, some hydrophilic residues such as aspartic acid, glutamic acid, and serine contribute significantly more favorably to protein solubility than other hydrophilic residues, such as asparagine, glutamine, threonine, lysine, and arginine.

B cell repertoire deep sequencing of human B cells from blood donors has been performed on a wide scale. Sequence information about a significant portion of the human antibody repertoire facilitates statistical assessment of antibody sequence features common in healthy humans. With knowledge about the antibody sequence features in a human recombined antibody variable gene reference database, the position specific degree of “Human Likeness” (HL) of an antibody sequence can be estimated. HL has been shown to be useful for the development of antibodies in clinical use, like therapeutic antibodies or antibodies as vaccines. The goal is to increase the human likeness of antibodies to reduce potential adverse effects and anti-antibody immune responses that will lead to significantly decreased efficacy of the antibody drug or can induce serious health implications. One can assess antibody characteristics of the combined antibody repertoire of three healthy human blood donors of about 400 million sequences in total and created a novel “relative Human Likeness” (rHL) score that focuses on the hypervariable region of the antibody. The rHL score allows one to easily distinguish between human (positive score) and non-human sequences (negative score). Antibodies can be engineered to eliminate residues that are not common in human repertoires.

Methods for reducing or eliminating the antigenicity of antibodies and antibody fragments are known in the art. When the antibodies are to be administered to a human, the antibodies preferably are “humanized” to reduce or eliminate antigenicity in humans. Preferably, each humanized antibody has the same or substantially the same affinity for the antigen as the non-humanized mouse antibody from which it was derived.

In one humanization approach, chimeric proteins are created in which mouse immunoglobulin constant regions are replaced with human immunoglobulin constant regions. See, e.g., Morrison et al., 1984, PROC. NAT. ACAD. SCI. 81:6851-6855, Neuberger et al., 1984, NATURE 312:604-608; U.S. Pat. No. 6,893,625 (Robinson); U.S. Pat. No. 5,500,362 (Robinson); and U.S. Pat. No. 4,816,567 (Cabilly).

Any suitable approach, including any of the above approaches, can be used to reduce or eliminate human immunogenicity of an antibody.

### H. Characterization of Antibodies

Antibodies according to the present disclosure may be defined, in the first instance, by their binding specificity. Those of skill in the art, by assessing the binding specificity/affinity of a given antibody using techniques well known to those of skill in the art, can determine whether such antibodies fall within the scope of the instant claims. For example, the epitope to which a given antibody binds may consist of a single contiguous sequence of 3 or more (e.g., 3, 4, 5, 6, 7, 8, 9, 10, 11, 12, 13, 14, 15, 16, 17, 18, 19, 20) amino acids located within the antigen molecule (e.g., a linear epitope in a domain). Alternatively, the epitope may consist of a plurality of non-contiguous amino acids (or amino acid sequences) located within the antigen molecule (e.g., a conformational epitope).

Various techniques known to persons of ordinary skill in the art can be used to determine whether an antibody “interacts with one or more amino acids” within a polypeptide or protein. Exemplary techniques include, for example, routine cross-blocking assays, such as that described in Antibodies, Harlow and Lane (Cold Spring Harbor Press, Cold Spring Harbor, N.Y.). Cross-blocking can be measured in various binding assays such as ELISA, biolayer interferometry, or surface plasmon resonance. Other methods include alanine scanning mutational analysis, peptide blot analysis (Reineke (2004) Methods Mol. Biol. 248: 443-63), peptide cleavage analysis, high-resolution electron microscopy techniques using single particle reconstruction, cryoEM, or tomography, crystallographic studies and NMR analysis. In addition, methods such as epitope excision, epitope extraction and chemical modification of antigens can be employed (Tomer (2000) Prot. Sci. 9: 487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water and exchangeable protons within amino acids that are protected by the antibody complex undergo deuterium-to-hydrogen back-exchange at a slower rate than exchangeable protons within amino acids that are not part of the interface. As a result, amino acids that form part of the protein/antibody interface may retain deuterium and therefore exhibit relatively higher mass compared to amino acids not included in the interface. After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267: 252-259; Engen and Smith (2001) Anal. Chem. 73: 256A-265A.

The term “epitope” refers to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation.

Modification-Assisted Profiling (MAP), also known as Antigen Structure-based Antibody Profiling (ASAP) is a method that categorizes large numbers of monoclonal antibodies directed against the same antigen according to the similarities of the binding profile of each antibody to chemically or enzymatically modified antigen surfaces (see US 2004/0101920, herein specifically incorporated by reference in its entirety). Each category may reflect a unique epitope either distinctly different from or partially overlapping with epitope represented by another category. This technology allows rapid filtering of genetically identical antibodies, such that characterization can be focused on genetically distinct antibodies. When applied to hybridoma screening, MAP may facilitate identification of rare hybridoma clones that produce monoclonal antibodies having the desired characteristics. MAP may be used to sort the antibodies of the disclosure into groups of antibodies binding different epitopes.

The present disclosure includes antibodies that may bind to the same epitope, or a portion of the same epitope. One can easily determine whether an antibody binds to the same epitope as, or competes for binding with, a reference antibody by using routine methods known in the art. For example, to determine if a test antibody binds to the same epitope as a reference antibody, the reference antibody is allowed to bind to the target molecule under saturating conditions. Next, the ability of a test antibody to bind to the target molecule is assessed. If the test antibody is able to bind to the target molecule following saturation binding with the reference antibody, it can be concluded that the test antibody binds to a different epitope than the reference antibody. On the other hand, if the test antibody is not able to bind to the target molecule following saturation binding with the reference antibody, then the test antibody may bind to the same epitope as the epitope bound by the reference antibody.

To determine if an antibody competes for binding with a reference antibody, the above-described binding methodology is performed in two orientations: In a first orientation, the reference antibody is allowed to bind to its antigen under saturating conditions followed by assessment of binding of the test antibody to the antigen. In a second orientation, the test antibody is allowed to bind to the antigen under saturating conditions followed by assessment of binding of the reference antibody to the antigen. If, in both orientations, only the first (saturating) antibody is capable of binding to the antigen, then it is concluded that the test antibody and the reference antibody compete for binding to the antigen. As will be appreciated by a person of ordinary skill in the art, an antibody that competes for binding with a reference antibody may not necessarily bind to the identical epitope as the reference antibody, but may sterically block binding of the reference antibody by binding an overlapping or adjacent epitope.

Two antibodies bind to the same or overlapping epitope if each competitively inhibits (blocks) binding of the other to the antigen. That is, a 1-, 5-, 10-, 20- or 100-fold excess of one antibody inhibits binding of the other by at least 50% but preferably 75%, 90%, or even 99% as measured in a competitive binding assay (see, e.g., Junghans et al., Cancer Res. 1990 50:1495-1502). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art.

In another aspect, the antibodies may be defined by their variable sequence, which include additional “framework” regions. These are provided in Tables 2, 3, 6, 9, and 10, that represent full variable regions. Furthermore, the antibodies sequences may vary from these sequences, optionally using methods discussed in greater detail below. For example, nucleic acid sequences may vary from those set out above in that (a) the variable regions may be segregated away from the constant domains of the light and heavy chains, (b) the nucleic acids may vary from those set out above while not affecting the residues encoded thereby, (c) the nucleic acids may vary from those set out above by a given percentage, e.g., 70%, 75%, 80%, 85%, 90%, 91%, 92%, 93%, 94%, 95%, 96%, 97%, 98% or 99% homology, (d) the nucleic acids may vary from those set out above by virtue of the ability to hybridize under high stringency conditions, as exemplified by low salt and/or high temperature conditions, such as provided by about 0.02 M to about 0.15 M NaCl at temperatures of about 50° C. to about 70° C., (e) the amino acids may vary from those set out above by a given percentage, e.g., 80%, 85%, 90%, 91%, 92%, 93%, 94%, 95%, 96%, 97%, 98% or 99% homology, or (f) the amino acids may vary from those set out above by permitting conservative substitutions.

When comparing polynucleotide and polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One particular example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides of the disclosure. Software for performing BLAST analyses is publicly available through the National Center for Biotechnology Information. The rearranged nature of an antibody sequence and the variable length of each gene requires multiple rounds of BLAST searches for a single antibody sequence. Also, manual assembly of different genes is difficult and error-prone. The sequence analysis tool IgBLAST (world-wide-web at ncbi.nlm.nih.gov/igblast/) identifies matches to the germline V, D and J genes, details at rearrangement junctions, the delineation of Ig V domain framework regions and complementarity determining regions. IgBLAST can analyze nucleotide or protein sequences and can process sequences in batches and allows searches against the germline gene databases and other sequence databases simultaneously to minimize the chance of missing possibly the best matching germline V gene.

In one approach, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residues occur in both sequences to yield the number of matched positions, dividing the number of matched positions by the total number of positions in the reference sequence (i.e., the window size) and multiplying the results by 100 to yield the percentage of sequence identity.

Yet another way of defining an antibody is as a “derivative” of any of the antibodies provided herein and their antigen-binding fragments. A derivative antibody or antibody fragment may be modified by chemical modifications using techniques known to those of skill in the art, including, but not limited to, specific chemical cleavage, acetylation, formulation, metabolic synthesis of tunicamycin, etc. In one embodiment, an antibody derivative will possess a similar or identical function as the parental antibody. In another embodiment, an antibody derivative will exhibit an altered activity relative to the parental antibody. For example, a derivative antibody (or fragment thereof) can bind to its epitope more tightly or be more resistant to proteolysis than the parental antibody.

The term “derivative” refers to an antibody or antigen-binding fragment thereof that immunospecifically binds to an antigen but which comprises, one, two, three, four, five or more amino acid substitutions, additions, deletions or modifications relative to a “parental” (or wild-type) molecule. Such amino acid substitutions or additions may introduce naturally occurring (i.e., DNA-encoded) or non-naturally occurring amino acid residues. The term “derivative” encompasses, for example, as variants having altered CH1, hinge, CH2, CH3 or CH4 regions, so as to form, for example antibodies, etc., having variant Fc regions that exhibit enhanced or impaired effector or binding characteristics. The term “derivative” additionally encompasses non-amino acid modifications, for example, amino acids that may be glycosylated (e.g., have altered mannose, 2-N-acetylglucosamine, galactose, fucose, glucose, sialic acid, 5-N-acetylneuraminic acid, 5-glycolneuraminic acid, etc. content), acetylated, pegylated, phosphorylated, amidated, derivatized by known protecting/blocking groups, proteolytic cleavage, linked to a cellular ligand or other protein, etc. In some embodiments, the altered carbohydrate modifications modulate one or more of the following: solubilization of the antibody, facilitation of subcellular transport and secretion of the antibody, promotion of antibody assembly, conformational integrity, and antibody-mediated effector function. In a specific embodiment, the altered carbohydrate modifications enhance antibody mediated effector function relative to the antibody lacking the carbohydrate modification. Carbohydrate modifications that lead to altered antibody mediated effector function are well known in the art.

One can determine the biophysical properties of antibodies. One can use elevated temperature to unfold antibodies to determine relative stability, using average apparent melting temperatures. Differential Scanning Calorimetry (DSC) measures the heat capacity, Cₚ, of a molecule (the heat required to warm it, per degree) as a function of temperature. One can use DSC to study the thermal stability of antibodies. DSC data for mAbs is particularly interesting because it sometimes resolves the unfolding of individual domains within the mAb structure, producing up to three peaks in the thermogram (from unfolding of the Fab, C_{H}2, and C_{H}3 domains). Typically unfolding of the Fab domain produces the strongest peak. The DSC profiles and relative stability of the Fc portion show characteristic differences for the human IgG₁, IgG₂, IgG₃, and IgG₄ subclasses (Garber and Demarest, Biochem. Biophys. Res. Commun. 355, 751-757, 2007). One also can determine average apparent melting temperature using circular dichroism (CD), performed with a CD spectrometer. Far-UV CD spectra will be measured for antibodies in the range of 200 to 260 nm at increments of 0.5 nm. The final spectra can be determined as averages of 20 accumulations. Residue ellipticity values can be calculated after background subtraction. Thermal unfolding of antibodies (0.1 mg/mL) can be monitored at 235 nm from 25-95° C. and a heating rate of 1° C./min. One can use dynamic light scattering (DLS) to assess for propensity for aggregation. DLS is used to characterize size of various particles including proteins. If the system is not disperse in size, the mean effective diameter of the particles can be determined. This measurement depends on the size of the particle core, the size of surface structures, and particle concentration. Since DLS essentially measures fluctuations in scattered light intensity due to particles, the diffusion coefficient of the particles can be determined. DLS software in commercial DLA instruments displays the particle population at different diameters. Stability studies can be done conveniently using DLS. DLS measurements of a sample can show whether the particles aggregate over time or with temperature variation by determining whether the hydrodynamic radius of the particle increases. If particles aggregate, one can see a larger population of particles with a larger radius. Stability depending on temperature can be analyzed by controlling the temperature in situ. Capillary electrophoresis (CE) techniques include proven methodologies for determining features of antibody stability. One can use an iCE approach to resolve antibody protein charge variants due to deamidation, C-terminal lysines, sialylation, oxidation, glycosylation, and any other change to the protein that can result in a change in pI of the protein. Each of the expressed antibody proteins can be evaluated by high throughput, free solution isoelectric focusing (IEF) in a capillary column (cIEF), using a Protein Simple Maurice instrument. Whole-column UV absorption detection can be performed every 30 seconds for real time monitoring of molecules focusing at the isoelectric points (pls). This approach combines the high resolution of traditional gel IEF with the advantages of quantitation and automation found in column-based separations while eliminating the need for a mobilization step. The technique yields reproducible, quantitative analysis of identity, purity, and heterogeneity profiles for the expressed antibodies. The results identify charge heterogeneity and molecular sizing on the antibodies, with both absorbance and native fluorescence detection modes and with sensitivity of detection down to 0.7 μg/mL.

One can determine the intrinsic solubility score of antibody sequences. The intrinsic solubility scores can be calculated using CamSol Intrinsic (Sormanni et al., J Mol Biol 427, 478-490, 2015). The amino acid sequences for residues 95-102 (Kabat numbering) in HCDR3 of each antibody fragment such as a scFv can be evaluated via the online program to calculate the solubility scores. One also can determine solubility using laboratory techniques. Various techniques exist, including addition of lyophilized protein to a solution until the solution becomes saturated and the solubility limit is reached, or concentration by ultrafiltration in a microconcentrator with a suitable molecular weight cut-off. The most straightforward method is induction of amorphous precipitation, which measures protein solubility using a method involving protein precipitation using ammonium sulfate (Trevino et al., J Mol Biol, 366: 449-460, 2007). Ammonium sulfate precipitation gives quick and accurate information on relative solubility values. Ammonium sulfate precipitation produces precipitated solutions with well-defined aqueous and solid phases and requires relatively small amounts of protein. Solubility measurements performed using induction of amorphous precipitation by ammonium sulfate also can be done easily at different pH values. Protein solubility is highly pH dependent, and pH is considered the most important extrinsic factor that affects solubility.

Generally, it is thought that autoreactive clones should be eliminated during ontogeny by negative selection; however, it has become clear that many human naturally occurring antibodies with autoreactive properties persist in adult mature repertoires, and the autoreactivity may enhance the antiviral function of many antibodies to pathogens. It has been noted that HCDR3 loops in antibodies during early B cell development are often rich in positive charge and exhibit autoreactive patterns (Wardemann et al., Science 301, 1374-1377, 2003). One can test a given antibody for autoreactivity by assessing the level of binding to human origin cells in microscopy (using adherent HeLa or HEp-2 epithelial cells) and flow cytometric cell surface staining (using suspension Jurkat T cells and 293S human embryonic kidney cells). Autoreactivity also can be surveyed using assessment of binding to tissues in tissue arrays.

### IV. Pharmaceutical Formulations

The present disclosure provides pharmaceutical compositions comprising antibodies or antibody fragments with engineered Fc domains. Such compositions comprise a prophylactically or therapeutically effective amount of an antibody or a fragment thereof and a pharmaceutically acceptable carrier. The present disclosure also provide pharmaceutical composition comprising a soluble gp34 protein. Such compositions can be used for stimulating an immune response, such as part of vaccine formulation. In some aspects, the pharmaceutical composition will comprise an mRNA encoding the therapeutic protein. In the case of an antibody therapeutic, methods of delivering mRNA encoding the antibody to the patient has been described, for example, in U.S. Pat. No. 10,899,830, which is incorporated herein by reference in its entirety.

In the case that a nucleic acid molecule encoding a soluble gp34 protein or antibody with an engineered Fc domain is used in a pharmaceutical composition, the nucleic acid molecule may comprise or consist of deoxyribonucleotides and/or ribonucleotides, or analogs thereof, covalently linked together. A nucleic acid molecule as described herein generally contains phosphodiester bonds, although in some cases, nucleic acid analogs are included that may have at least one different linkage, e.g., phosphoramidate, phosphorothioate, phosphorodithioate, or O-methylphosphoroamidite linkages, and peptide nucleic acid backbones and linkages. Mixtures of naturally occurring polynucleotides and analogs can be made; alternatively, mixtures of different polynucleotide analogs, and mixtures of naturally occurring polynucleotides and analogs may be made. A nucleic acid molecule may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, the term polynucleotide encompasses both the double-stranded form and each of two complementary single-stranded forms known or predicted to make up the double-stranded form. A nucleic acid molecule is composed of a specific sequence of four nucleotide bases: adenine (A), cytosine (C), guanine (G), thymine (T), and uracil (U) for thymine when the polynucleotide is RNA. Thus, the term “nucleic acid sequence” is the alphabetical representation of a nucleic acid molecule. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues.

In some embodiments, the nucleic acids of the present disclosure comprise one or more modified nucleosides comprising a modified sugar moiety. Such compounds comprising one or more sugar-modified nucleosides may have desirable properties, such as enhanced nuclease stability or increased binding affinity with a target nucleic acid relative to an oligonucleotide comprising only nucleosides comprising naturally occurring sugar moieties. In some embodiments, modified sugar moieties are substituted sugar moieties. In some embodiments, modified sugar moieties are sugar surrogates. Such sugar surrogates may comprise one or more substitutions corresponding to those of substituted sugar moieties.

In some embodiments, modified sugar moieties are substituted sugar moieties comprising one or more non-bridging sugar substituent, including but not limited to substituents at the 2′ and/or 5′ positions. Examples of sugar substituents suitable for the 2′-position, include, but are not limited to: 2′-F, 2′-OCH3 (“OMe” or “O-methyl”), and 2′-O(CH2)2OCH3 (“MOE”). In certain embodiments, sugar substituents at the 2′ position is selected from allyl, amino, azido, thio, O-allyl, O—C1-C10 alkyl, O—C1-C10 substituted alkyl; OCF3, O(CH2)2SCH3, O(CH2)2-O—N(Rm)(Rn), and O—CH2-C(═O)—N(Rm)(Rn), where each Rm and Rn is, independently, H or substituted or unsubstituted C₁-C₁₀ alkyl. Examples of sugar substituents at the 5′-position, include, but are not limited to: 5′-methyl (R or S); 5′-vinyl, and 5′-methoxy. In some embodiments, substituted sugars comprise more than one non-bridging sugar substituent, for example, T-F-5′-methyl sugar moieties (see, e.g., PCT International Application WO 2008/101157, for additional 5′,2′-bis substituted sugar moieties and nucleosides).

Nucleosides comprising 2′-substituted sugar moieties are referred to as 2′-substituted nucleosides. In some embodiments, a 2′-substituted nucleoside comprises a 2′-substituent group selected from halo, allyl, amino, azido, SH, CN, OCN, CF3, OCF3, O, S, or N(Rm)-alkyl; O, S, or N(Rm)-alkenyl; O, S or N(Rm)-alkynyl; O-alkylenyl-O-alkyl, alkynyl, alkaryl, aralkyl, O-alkaryl, O-aralkyl, O(CH2)2SCH3, O(CH2)2-O—N(Rm)(Rn) or O—CH2-C(═O)—N(Rm)(Rn), where each Rm and Rn is, independently, H, an amino protecting group or substituted or unsubstituted C1-C10 alkyl. These 2′-substituent groups can be further substituted with one or more substituent groups independently selected from hydroxyl, amino, alkoxy, carboxy, benzyl, phenyl, nitro (NO2), thiol, thioalkoxy (S-alkyl), halogen, alkyl, aryl, alkenyl and alkynyl.

In some embodiments, a 2′-substituted nucleoside comprises a 2′-substituent group selected from F, NH2, N3, OCF3, O—CH3, O(CH2)3NH2, CH2-CH═CH2, O—CH2-CH═CH2, OCH2CH2OCH3, O(CH2)2SCH3, O—(CH2)2-O—N(Rm)(Rn), O(CH2)2O(CH2)2N(CH3)2, and N-substituted acetamide (O—CH2-C(═O)—N(Rm)(Rn) where each Rm and Rn is, independently, H, an amino protecting group or substituted or unsubstituted C₁-C₁₀ alkyl.

In some embodiments, a 2′-substituted nucleoside comprises a sugar moiety comprising a 2′-substituent group selected from F, OCF3, O—CH3, OCH2CH2OCH3, O(CH2)2SCH3, O(CH2)2-O—N(CH3)2, —O(CH2)2O(CH2)2N(CH3)2, and O—CH2-C(═O)—N(H)CH3.

In some embodiments, a 2′-substituted nucleoside comprises a sugar moiety comprising a 2′-substituent group selected from F, O—CH3, and OCH2CH2OCH3.

In some embodiments, nucleosides of the present disclosure comprise one or more unmodified nucleobases. In certain embodiments, nucleosides of the present disclosure comprise one or more modified nucleobases.

In some embodiments, modified nucleobases are selected from: universal bases, hydrophobic bases, promiscuous bases, size-expanded bases, and fluorinated bases as defined herein. 5-substituted pyrimidines, 6-azapyrimidines and N-2, N-6 and O-6 substituted purines, including 2-aminopropyladenine, 5-propynyluracil; 5-propynylcytosine; 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, 3-deazaguanine and 3-deazaadenine, universal bases, hydrophobic bases, promiscuous bases, size-expanded bases, and fluorinated bases as defined herein. Further modified nucleobases include tricyclic pyrimidines such as phenoxazine cytidine([5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g., 9-(2-aminoethoxy)-H-pyrimido[5,4-13][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, Kroschwitz, J. I., Ed., John Wiley & Sons, 1990, 858-859.

Representative United States patents that teach the preparation of certain of the above noted modified nucleobases as well as other modified nucleobases include without limitation, U.S. Pat. Nos. 3,687,808; 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121; 5,596,091; 5,614,617; 5,645,985; 5,681,941; 5,750,692; 5,763,588; 5,830,653 and 6,005,096, each of which is herein incorporated by reference in its entirety.

Additional modifications may also be made at other positions on the oligonucleotide, particularly the 3′ position of the sugar on the 3′ terminal nucleotide and the 5′ position of 5′ terminal nucleotide. For example, one additional modification of the ligand conjugated oligonucleotides of the present disclosure involves chemically linking to the oligonucleotide one or more additional non-ligand moieties or conjugates which enhance the activity, cellular distribution or cellular uptake of the oligonucleotide. Such moieties include but are not limited to lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety. In some aspects, a nucleic acid molecule encoding a soluble gp34 or engineered antibody is a modified RNA, such as, for example, a modified mRNA. Modified (m)RNA contemplates certain chemical modifications that confer increased stability and low immunogenicity to mRNAs, thereby facilitating expression of therapeutically important proteins. For instance, N1-methyl-pseudouridine (N1mΨ) outperforms several other nucleoside modifications and their combinations in terms of translation capacity. In some embodiments, the (m)RNA molecules used herein may have the uracils replaced with psuedouracils such as 1-methyl-3′-pseudouridylyl bases. In some embodiments, some of the uracils are replaced, but in other embodiments, all of the uracils have been replaced. The (m)RNA may comprise a 5′ cap, a 5′ UTR element, an optionally codon optimized open reading frame, a 3′ UTR element, and a poly(A) sequence and/or a polyadenylation signal.

The nucleic acid molecule, whether native or modified, may be delivered as a naked nucleic acid molecule or in a delivery vehicle, such as a lipid nanoparticle. A lipid nanoparticle may comprise one or more nucleic acids present in a weight ratio to the lipid nanoparticles from about 5:1 to about 1:100. In some embodiments, the weight ratio of nucleic acid to lipid nanoparticles is from about 5:1, 2.5:1, 1:1, 1:5, 1:10, 1:15, 1:20, 1:25, 1:30, 1:35, 1:40, 1:45, 1:50, 1:60, 1:70, 1:80, 1:90, or 1:100, or any value derivable therein.

In some embodiments, the lipid nanoparticles used herein may contain one, two, three, four, five, six, seven, eight, nine, or ten lipids. These lipids may include triglycerides, phospholipids, steroids or sterols, a PEGylated lipids, or a group with a ionizable group such as an alkyl amine and one or more hydrophobic groups such as C6 or greater alkyl groups.

In some aspects of the present disclosure, the lipid nanoparticles are mixed with one or more steroid or a steroid derivative. In some embodiments, the steroid or steroid derivative comprises any steroid or steroid derivative. As used herein, in some embodiments, the term “steroid” is a class of compounds with a four ring 17 carbon cyclic structure, which can further comprises one or more substitutions including alkyl groups, alkoxy groups, hydroxy groups, oxo groups, acyl groups, or a double bond between two or more carbon atoms.

In some aspects of the present disclosure, the lipid nanoparticles are mixed with one or more PEGylated lipids (or PEG lipid). n some embodiments, the present disclosure comprises using any lipid to which a PEG group has been attached. In some embodiments, the PEG lipid is a diglyceride which also comprises a PEG chain attached to the glycerol group. In other embodiments, the PEG lipid is a compound which contains one or more C6-C24 long chain alkyl or alkenyl group or a C6-C24 fatty acid group attached to a linker group with a PEG chain. Some non-limiting examples of a PEG lipid includes a PEG modified phosphatidylethanolamine and phosphatidic acid, a PEG ceramide conjugated, PEG modified dialkylamines and PEG modified 1,2-diacyloxypropan-3-amines, PEG modified diacylglycerols and dialkylglycerols. In some embodiments, PEG modified diastearoylphosphatidylethanolamine or PEG modified dimyristoyl-sn-glycerol. In some embodiments, the PEG modification is measured by the molecular weight of PEG component of the lipid. In some embodiments, the PEG modification has a molecular weight from about 100 to about 15,000. In some embodiments, the molecular weight is from about 200 to about 500, from about 400 to about 5,000, from about 500 to about 3,000, or from about 1,200 to about 3,000. The molecular weight of the PEG modification is from about 100, 200, 400, 500, 600, 800, 1,000, 1,250, 1,500, 1,750, 2,000, 2,250, 2,500, 2,750, 3,000, 3,500, 4,000, 4,500, 5,000, 6,000, 7,000, 8,000, 9,000, 10,000, 12,500, to about 15,000. Some non-limiting examples of lipids that may be used in the present disclosure are taught by U.S. Pat. No. 5,820,873, WO 2010/141069, or U.S. Pat. No. 8,450,298, which is incorporated herein by reference.

In some aspects of the present disclosure, the lipid nanoparticles are mixed with one or more phospholipids. In some embodiments, any lipid which also comprises a phosphate group. In some embodiments, the phospholipid is a structure which contains one or two long chain C6-C24 alkyl or alkenyl groups, a glycerol or a sphingosine, one or two phosphate groups, and, optionally, a small organic molecule. In some embodiments, the small organic molecule is an amino acid, a sugar, or an amino substituted alkoxy group, such as choline or ethanolamine. In some embodiments, the phospholipid is a phosphatidylcholine. In some embodiments, the phospholipid is distearoylphosphatidylcholine or dioleoylphosphatidylethanolamine. In some embodiments, other zwitterionic lipids are used, where zwitterionic lipid defines lipid and lipid-like molecules with both a positive charge and a negative charge.

In some aspects of the present disclosure, lipid nanoparticle containing compounds containing lipophilic and cationic components, wherein the cationic component is ionizable, are provided. In some embodiments, the cationic ionizable lipids contain one or more groups which is protonated at physiological pH but may deprotonated and has no charge at a pH above 8, 9, 10, 11, or 12. The ionizable cationic group may contain one or more protonatable amines which are able to form a cationic group at physiological pH. The cationic ionizable lipid compound may also further comprise one or more lipid components such as two or more fatty acids with C6-C24 alkyl or alkenyl carbon groups. These lipid groups may be attached through an ester linkage or may be further added through a Michael addition to a sulfur atom. In some embodiments, these compounds may be a dendrimer, a dendron, a polymer, or a combination thereof.

In some aspects of the present disclosure, composition containing compounds containing lipophilic and cationic components, wherein the cationic component is ionizable, are provided. In some embodiments, ionizable cationic lipids refer to lipid and lipid-like molecules with nitrogen atoms that can acquire charge (pKa). These lipids may be known in the literature as cationic lipids. These molecules with amino groups typically have between 2 and 6 hydrophobic chains, often alkyl or alkenyl such as C6-C24 alkyl or alkenyl groups, but may have at least 1 or more that 6 tails.

In some embodiments, the amount of the lipid nanoparticle with the nucleic acid molecule encapsulated in the pharmaceutical composition is from about 0.1% w/w to about 50% w/w, from about 0.25% w/w to about 25% w/w, from about 0.5% w/w to about 20% w/w, from about 1% w/w to about 15% w/w, from about 2% w/w to about 10% w/w, from about 2% w/w to about 5% w/w, or from about 6% w/w to about 10% w/w. In some embodiments, the amount of the lipid nanoparticle with the nucleic acid molecule encapsulated in the pharmaceutical composition is from about 0.1% w/w, 0.25% w/w, 0.5% w/w, 1% w/w, 2.5% w/w, 5% w/w, 7.5% w/w, 10% w/w, 15% w/w, 20% w/w, 25% w/w, 30% w/w, 35% w/w, 40% w/w, 45% w/w, 50% w/w, 55% w/w, 60% w/w, 65% w/w, 70% w/w, 75% w/w, 80% w/w, 85% w/w, 90% w/w, to about 95% w/w, or any range derivable therein.

In some aspects, the present disclosure comprises one or more sugars formulated into pharmaceutical compositions. In some embodiments, the sugars used herein are saccharides. These saccharides may be used to act as a lyoprotectant that protects the pharmaceutical composition from destabilization during the drying process. These water-soluble excipients include carbohydrates or saccharides such as disaccharides such as sucrose, trehalose, or lactose, a trisaccharide such as fructose, glucose, galactose comprising raffinose, polysaccharides such as starches or cellulose, or a sugar alcohol such as xylitol, sorbitol, or mannitol. In some embodiments, these excipients are solid at room temperature. Some non-limiting examples of sugar alcohols include erythritol, threitol, arabitol, xylitol, ribitol, mannitol, sorbitol, galactitol, fucitol, iditol, inositol, volemitol, isomalt, maltitol, lactitol, maltotritol, maltotetraitol, or a polyglycitol.

In some embodiments, the amount of the sugar in the pharmaceutical composition is from about 25% w/w to about 98% w/w, 40% w/w to about 95% w/w, 50% w/w to about 90% w/w, 50% w/w to about 70% w/w, or from about 80% w/w to about 90% w/w. In some embodiments, the amount of the sugar in the pharmaceutical composition is from about 10% w/w, 15% w/w, 20% w/w, 25% w/w, 30% w/w, 35% w/w, 40% w/w, 45% w/w, 50% w/w, 52.5% w/w, 55% w/w, 57.5% w/w, 60% w/w, 62.5% w/w, 65% w/w, 67.5% w/w, 70% w/w, 75% w/w, 80% w/w, 82.5% w/w, 85% w/w, 87.5% w/w, 90% w/w, to about 95% w/w, or any range derivable therein.

In some embodiments, the pharmaceutically acceptable polymer is a copolymer. The pharmaceutically acceptable polymer may further comprise one, two, three, four, five, or six subunits of discrete different types of polymer subunits. These polymer subunits may include polyoxypropylene, polyoxyethylene, or a similar subunit. In particular, the pharmaceutically acceptable polymer may comprise at least one hydrophobic subunit and at least one hydrophilic subunit. In particular, the copolymer may have hydrophilic subunits on each side of a hydrophobic unit. The copolymer may have a hydrophilic subunit that is polyoxyethylene and a hydrophobic subunit that is polyoxypropylene.

In some aspects, the present disclosure provides pharmaceutical compositions that contain one or more salts. The salts may be an inorganic potassium or sodium salt such as potassium chloride, sodium chloride, potassium phosphate dibasic, potassium phosphate monobasic, sodium phosphate dibasic, or sodium phosphate monobasic. The pharmaceutical composition may comprise one or more phosphate salts such to generate a phosphate buffer solution. The phosphate buffer solution may be comprise each of the phosphates to buffer a solution to a pH from about 6.8, 6.9, 7.0, 7.1, 7.2, 7.3, 7.4, 7.5, 7.6, 7.7, 7.8, 7.9, or 8.0, or any range derivable therein.

In some aspects, the present disclosure comprises one or more excipients formulated into pharmaceutical compositions. An “excipient” refers to pharmaceutically acceptable carriers that are relatively inert substances used to facilitate administration or delivery of an API into a subject or used to facilitate processing of an API into drug formulations that can be used pharmaceutically for delivery to the site of action in a subject. Furthermore, these compounds may be used as diluents in order to obtain a dosage that can be readily measured or administered to a patient. Non-limiting examples of excipients include polymers, stabilizing agents, surfactants, surface modifiers, solubility enhancers, buffers, encapsulating agents, antioxidants, preservatives, nonionic wetting or clarifying agents, viscosity increasing agents, and absorption-enhancing agents.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and can preferably include an adjuvant. Water is a particular carrier when the pharmaceutical composition is administered by injections, such an intramuscular injection. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in “Remington's Pharmaceutical Sciences.” Such compositions will contain a prophylactically or therapeutically effective amount of the antibody or fragment thereof, preferably in purified form, together with a suitable amount of carrier so as to provide the form for proper administration to the patient. The formulation should suit the mode of administration, which can be oral, intravenous, intraarterial, intrabuccal, intranasal, nebulized, bronchial inhalation, or delivered by mechanical ventilation.

Therapeutic proteins and mRNAs of the present disclosure, as described herein, can be formulated for parenteral administration, e.g., formulated for injection via the intradermal, intravenous, intramuscular, subcutaneous, intra-tumoral or even intraperitoneal routes. The antibodies could alternatively be administered by a topical route directly to the mucosa, for example by nasal drops, inhalation, or by nebulizer. Pharmaceutically acceptable salts include the acid salts and those which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Generally, the ingredients of compositions of the disclosure are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Dosage can be by a single dose schedule or a multiple dose schedule. Multiple doses may be used in a primary immunization schedule and/or in a booster immunization schedule. In a multiple dose schedule the various doses may be given by the same or different routes. Multiple doses will typically be administered at least 1 week apart (e.g., about 2 weeks, about 3 weeks, about 4 weeks, about 6 weeks, about 8 weeks, about 10 weeks, about 12 weeks, about 16 weeks, etc.).

The compositions disclosed herein may be used to treat both children and adults. Thus, a human subject may be less than 1 year old, 1-5 years old, 5-16 years old, 16-55 years old, 55-65 years old, or at least 65 years old.

Preferred routes of administration include, but are not limited to, intramuscular, intraperitoneal, intradermal, subcutaneous, intravenous, intraarterial, and intraoccular injection. Particularly preferred routes of administration include intramuscular, intradermal and subcutaneous injection.

### V. Methods of Treatment

The recombinant antibodies provided herein can be used to prevent or treat a disease or disorder, such as a an HCMV infection or an HCMV infection-related disorder, which may comprise administering to a patient in need thereof an effective amount of one or more recombinant antibody as described herein alone or in a combined therapeutic regimen with another appropriate medicament known in the art or described herein.

Human cytomegalovirus (HCMV) is a genus of viruses in the order Herpesvirales, in the family Herpesviridae, in the subfamily Betaherpesvirinae. There are currently eight species in this genus, which have been identified and classified for different mammals, including humans, monkeys, and rodents. The most studied genus is human cytomegalovirus, also known as human herpesvirus 5 (HHV-5), which is widely distributed in the human population. Diseases associated with HHV-5 include mononucleosis and pneumonias. All herpesviruses share a characteristic ability to remain latent within the body over long periods of time. Although they may be found throughout the body, CMV infections are frequently associated with the salivary glands in humans and other mammals. Other CMV viruses are found in several mammal species, but species isolated from animals differ from HCMV in terms of genomic structure, and have not been reported to cause human disease.

Primary infection normally results in subclinical disease after which the virus becomes latent, retaining the capacity to reactivate at a later time. The virus is transmitted through body fluids, such as blood, saliva, urine, semen and breast milk. In particular, individuals with undeveloped or compromised immunity are highly sensitive to infection by HCMV. It is estimated that at least 60% of the US population has been exposed to CMV, with a prevalence of more than 90% in high-risk groups (e.g., unborn babies whose mothers become infected with CMV during the pregnancy or people with HIV).

In healthy individuals, HCMV typically causes an asymptomatic infection or produces mild, flulike symptoms. However, among two populations, HCMV is responsible for serious medical conditions. First, HCMV is a major cause of congenital defects in newborns infected in utero. Among congenitally infected newborns, 5-10% have major clinical symptoms at birth, such as microcephaly, intracranial calcifications, and hepatitis, as well as cytomegalic inclusion disease, which affects many tissues and organs including the central nervous system, liver, and retina and can lead to multi-organ failure and death. Other infants may be asymptomatic at birth, but later develop hearing loss or central nervous system abnormalities causing, in particular, poor intellectual performance and mental retardation. These pathologies are due in part to the ability of HCMV to enter and replicate in diverse cell types including epithelial cells, endothelial cells, smooth muscle cells, fibroblasts, neurons, and monocytes/macrophages.

The second population at risk are immunocompromised patients, such as those suffering from HIV infection and those undergoing transplantations. In this situation, the virus becomes an opportunistic pathogen and causes severe disease with high morbidity and mortality. The clinical disease causes a variety of symptoms including fever, pneumonia, hepatitis, encephalitis, myelitis, colitis, uveitis, retinitis, and neuropathy. Rarer manifestations of HCMV infections in immunocompetent individuals include Guillain-Barré syndrome, meningoencephalitis, pericarditis, myocarditis, thrombocytopenia, and hemolytic anemia. Moreover, HCMV infection increases the risk of organ graft loss through transplant vascular sclerosis and restenosis, and may increase atherosclerosis in transplant patients as well as in the general population. It is estimated that HCMV infection causes clinical disease in 75% of patients in the first year after transplantation.

In addition, antibody therapy has been used to control HCMV infection in immunocompromised individuals and to reduce the pathological consequences of maternal-fetal transmission, although such therapy is usually not sufficient to eradicate the virus. HCMV immunoglobulins (Igs) have been administered to transplant patients in association with immunosuppressive treatments for prophylaxis of HCMV disease with mixed results. Antibody therapy has also been used to control brief infection and prevent disease in newborns. However, these products are plasma derivatives with relatively low potency and have to be administered by intravenous infusion at very high doses in order to deliver sufficient amounts of neutralizing antibodies.

The recombinant antibodies provided herein are useful as therapeutic agents in the treatment of diseases or disorders involving HCMV infection and/or activity, especially resulting from high viral load in a patient. A method of treatment may comprise administering an effective amount of a recombinant antibody to a patient in need thereof, wherein aberrant infection and/or activity of HCMV is decreased. A method of treatment may comprise (i) identifying a patient demonstrating HCMV infection levels or activity, and (ii) administering an effective amount of a recombinant antibody provided herein to the patient, wherein expression and/or activity of HCMV is decreased. An effective amount according to the invention is an amount that decreases the expression and/or activity of HCMV so as to decrease or lessen the severity of at least one symptom of the HCMV infection or particular disease or disorder being treated, but not necessarily cure the disease or disorder.

There is evidence for involvement of HCMV infection in a variety of disorders. Such disorders may affect immunocompromised patients, such as allograft recipients and HIV infected individuals, and may include for example: fever, hepatitis, retinitis, pneumonitis, myelosuppression, encephalopathy, polyradiculopathy, immunosuppression, rejection/graft-versus-host disease or atherosclerosis. A recombinant antibody provided herein may also be used to treat intra-uterine infection in neonates. Frequently, neonates are born without signs or symptoms of the disorders listed above, but without treatment may develop progressive symptoms of CNS dysfunction and impairment, e.g. but not limited to hearing loss, loss of vision, and/or mental retardation.

“Treatment” and “treating” refer to administration or application of a therapeutic agent to a subject or performance of a procedure or modality on a subject for the purpose of obtaining a therapeutic benefit of a disease or health-related condition. For example, a treatment may include administration of a pharmaceutically effective amount of an antibody or antibody fragment with an engineered Fc domain, either alone or in combination with administration of an additional therapeutic.

The term “subject” as used herein refers to any individual or patient to which the subject methods are performed. Generally, the subject is human, although as will be appreciated by those in the art, the subject may be an animal. Thus, other animals, including mammals, such as rodents (including mice, rats, hamsters, and guinea pigs), cats, dogs, rabbits, farm animals (including cows, horses, goats, sheep, pigs, etc.), and primates (including monkeys, chimpanzees, orangutans, and gorillas) are included within the definition of subject.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of HCMV may involve, for example, a reduction in the viral load.

### VI. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Materials and Methods

Cells lines. CHO-K1 (CCL-61), MRC5 fibroblasts (CCL-171), and SKOV3 cells (HTB-77) were purchased from ATCC and maintained in complete medium (DMEM, 10% FBS, and 100 U/mL Penicillin) at 37° C./5% CO₂. NK-92 cells were purchased from ATCC as well with both the V158 & F158 alleles (PTA-8836 & 8837 respectively) and maintained in α-MEM media (12.5% FBS (Gibco), 12.5% horse serum (Thermo Fisher Scientific), 0.2 mM Myo-inositol (Sigma), 0.1 mM β-mercaptoethanol (Sigma, #636869), 0.02 mM folic acid (Sigma), 1.5 g/L sodium bicarbonate, 1 mM non-essential amino acids (Gibco), 1 mM sodium pyruvate (Gibco #11360-039), 2 mM glutamine, supplemented with 200 rU/mL of IL2 (Sigma), maintained at 37° C./5% CO₂. Human monocytic cells line, THP-1 (ATCC TIB-202), was a gift from the George Georgiou Lab and were maintained in RPMI media (10% FBS, 100 U/mL Penicillin) and also maintained at 37° C./5% CO₂. ExpiCHO (A29133) and Expi293 cells (A41249) were purchased from Thermo Scientific and maintained in ExpiCHO expression medium and Expi293 expression medium at 37° C., at 5% and 8% CO₂ respectively. BW5147 mouse thymoma cells (kindly provided by Ofer Mandelboim, Hadassah Hospital, Jerusalem, Israel) were maintained at 3×10⁵ to 9×10⁵ cells/ml in RPMI (RPMI GlutaMAX, Gibco #61870-010) supplemented with 10% FBS, sodium pyruvate (1×, Gibco #11360-039) and β-mercaptoethanol (0.1 mM, Sigma #636869). Cells were maintained at 37° C./5% CO₂.

Virus stocks. The following stocks were produced and used throughout the studies: BAC2-AD169 GFP (gift from Professor Thomas Shenk, Princeton University); BAC2-AD169-varL; BAC2-AD169-varL Δgp34 Δgp68 Δgp95 (ΔΔΔ). Deletion virus mutants were generated as described previously (Kolb et al., 2021). In brief, recombinant HCMV mutants were generated according to previously published procedures (Karstentischer et al., 2006, Wagner et al., 2002) using pAD169-BAC2 (MN900952.1, (Le-Trilling et al., 2020)) corresponding to AD169varL (Le et al., 2011) as parental genome. For the construction of the HCMV deletion mutants, a PCR fragment was generated using the plasmid pSLFRTKn (Atalay et al., 2002) as the template DNA. The PCR fragment containing a kanamycin resistance gene was inserted into the parental BAC by homologous recombination in E. coli. The inserted cassette replaces the target sequence which was defined by flanking sequences in the primers. This cassette is flanked by frt-sites which can be used to remove the kanamycin resistance gene by FLP-mediated recombination. The removal of the cassette results in a single remaining frt-site. The deletion of multiple non-adjacent genes was conducted in consecutive steps.

The gene TRL11 was deleted by use of the primers KL-DeltaTRL11-Kana1
(ACGACGAAGAGGACGAGGACGACAACGTCTGATAAGGAAGGCGAGAACGTGTTTTGCACCCC
AGTGAATTCGAGCTCGGTAC; SEQ ID NO: 21)
and
KL-DeltaTRL11-Kana2
(TGTATACGCCGTATGCCTGTACGTGAGATGGTGAGGTCTTCGGCAGGCGACACGCATCTTGA
CCATGATTACGCCAAGCTCC; SEQ ID NO: 22).
The gene TRL12 was deleted by use of the primers KL-DeltaTRL12-Kana1
(CGGACGGACCTAGATACGGAACCTTTGTTGTTGACGGTGGACGGGGATTTACAGTAAAAGCC
AGTGAATTCGAGCTCGGTAC; SEQ ID NO: 23)
and
KL-DeltaTRL12-Kana2
(CCTTACAGAATGTTTTAGTTTATTGTTCAGCTTCATAAGATGTCTGCCCGGAAACGTAGCGA
CCATGATTACGCCAAGCTCC; SEQ ID NO: 24).
The gene UL119 was deleted by use of the primers KL-DeltaUL119-Kana1
(TTGTTTATTTTGTTGGCAGGTTGGCGGGGGAGGAAAAGGGGTTGAACAGAAAGGTAGGTGCC
AGTGAATTCGAGCTCGGTAC; SEQ ID NO: 25)
and
KL-DeltaUL119-Kana2
(AGGTGACGCGACCTCCTGCCACATATAGCTCGTCCACACGCCGTCTCGTCACACGGCAACGA
CCATGATTACGCCAAGCTCC; SEQ ID NO: 26).

HCMV infection. For staining and in vitro assay experiments, MRC5 fibroblasts were cultured in complete medium (DMEM (Gibco), 10% Fetal Bovine Serum, 100 U/ml Penicillin) to 80% confluency in 6 well plates prior to infection with HCMV AD169-GFP at an MOI=2. Media was not changed, and virus was allowed to incubate 72-96 hours prior to performance of experiments. AD169-GFP BAC was a gift from the Shenk Lab and was used to generate viral stocks as in methods described. To generate stocks from AD169-GFP virus, T-175 flasks were cultured with MRC5 cells up to 80% confluency and then infected with virus at an MOI=0.1. One hour after infection the virus-containing media was removed and replaced with fresh complete media. Infected cells were incubated for 11-14 days at 37° C./5% CO₂ prior to harvesting supernatant and concentrating virus in media using 20% sorbitol cushion. Pellet was resuspended in filter sterilized in 7% sucrose/1% BSA/PBS and aliquots were stored at −80° C. Concentration of stored virus is determined using plaque assay or limiting dilution method.

Preparation of recombinant proteins. UL118-UL119 (gp68) was acquired through Uniprot (P16809) and was codon optimized (IDT). RL11 (gp34, Uniprot 16809) was cloned from the AD169-GFP BAC plasmid (a gift from the Thomas Shenk Lab at Princeton University). Both receptors (gp68 68-289 and gp34 24-182) were cloned into the pcDNA3.0 vector with a c-term FLAG tag (M2) and single or twin strep tag. All DNA oligos were purchased from IDT. CHO cells were then used to produce soluble gp34 constructs, cells were transfected in low-IgG medium (10% low IgG FBS+DMEM) using Lipofectamine 2000. Day 1 of transfection cells were incubated at 37° C./5% CO₂ and after 24 hours media was changed and cells were allowed to express protein at 32° C./5% CO₂ for an additional 4 days. Media was then harvested, and protein was isolated using Streptactin XT column (IBA Life Sciences) on AKTA Pure FPLC. Wash buffer consisted of 100 mM Tris-HCl (Sigma), 150 mM NaCl (sigma), pH 8.0, and elution buffer was the same as wash buffer plus 50 mM biotin (Sigma). ExpiCHO high-yield cells were used to produce soluble gp68 constructs. Manufacture's transfection guidelines were used; however, cells were only allowed to express gp68 for maximum of 5 days at 32° C./5% CO₂. ExpiCHO supernatant was harvested and blocked with 1 U avidin per μg of biotin in media. Proteins were purified via Streptactin XT column on AKTA Pure FPLC (GE Healthcare) as previously described. All proteins were buffer exchanged into PBS using Amicon Ultra-30 centrifugal spin columns (Millipore) and aliquoted and frozen down at −80° C.

| Protein | Sequence |
|---|---|
| gp34 | MQTYSTPLTLVIVTSLFLFTTQGSSSNAVEPTKKPLKLANYRATCEDRTRT |
| | LVTRLNTSHHSVVWQRYDIYSRYMRRMPPLCIITDAYKETTRQGGAAFACT |
| | RQNLTLYNLTVKDTGVYLLQDQYTGDVEAFYLIIHPRSFCRALETRRCFYP |
| | GPGRVVVTDSQEADRAIISDLKRQWSGLSLHCAWVSGMMIFVGALVICFLR |
| | SQRIGEQDAEHLRTDLDTEPLLLTVDGDLQ (SEQ ID NO: 11) |
| gp68 | MCPALAIALAAALLSNTHPGMGSSTTSAVTSPNITVTFTTSINTPNNVTST |
| | VTTTVQTSTFTSGSTSTSASTSVATTTQKEGHLYNVNCEASYSYDQVSLNA |
| | TCKVNLLNNTKNPDILSVTCYARTNCKGPFTQVGYLSAFPSDDKGKLHLSY |
| | NATAQELLISGLRPQETTEYTCSFFSWGRHHNATWDLFTYPIYAVYGTRLN |
| | ATTMRVRVLLQEHEHCLINGSSLYHPNSTVHLHQGNQLIPPWNISNVTYNG |
| | QRLREFVFYLNGTYTVVRLHVQIAGRSFTTTYVFIKSDPLFEDRLLAYGVL |
| | AFLVFMVIILLYVTYMLARRRDWSYKRLEEPVEEKKHPVPYFKQW (SEQ |
| | ID NO: 12) |
| Merlin RL13 | MHWHLAITWTVIILTFSECYNQTCPCPCICVNSTTVSISTSETTSKNITPT |
| | TTTNSKKTTSSIATTTPSLVTTGKVVSTAASSTIISQTNRSHTSNAITTPK |
| | TRFEYNITGYVGQEVTLNFTGSWNYIQWFRYGSPGWLYSSEPICTVTSNYH |
| | HTFPRGALCFDCDMTKLLIYDLTLNDSGKYVVKRTRHDNQYEEACYSLTVI |
| | FANTTSIVTNRTCDRKRTENTDTTNHEIGKHIIETIKKANIPLGIHAVWAG |
| | IVVSVALIALYMGNRRRPRKPRYTRLPKYDPDESWTKT (SEQ ID NO: |
| | 13) |
| AD169 RL12 | MRVACRRPHHLTYRHTAYTIIIFYILHRVTCNSTTINTASITSPNTASTTF |
| | VTSVFSTPNNNTSTTPHTSVTSQASTIGNITNVTSDLSTFTTVYSTFNTSY |
| | ANISNTAATTELISTNTNTILSFTNVTANATSSYNTTITVTITSDETSHNV |
| | STNTALISTPWLTNCSATTYTTYNRTNSSNACHTETTIIRFKETNTTGIEG |
| | SNVTIKGNSTWDCLSVAWIRHYNRSTHGHHLGHRKNAHTQSWYWLRILTSH |
| | TVCHSQHERPSLYHDLCRSCNNTELHLYDLNITNSGRYSRRCFKENYFTGH |
| | HEDENFYLLVTPKNHTEAINATFVCPRYNTDIENEDREKGSQHTNNTHHHK |
| | RNLYHSSQRSRTVWTIVLVCMACIVLFFARRAFNKKYHMLQDTVSESEFIV |
| | RYHTEHED (SEQ ID NO: 14) |
| Merlin RL12 | MRVDRQKRNNFTYRQTVYIILTFYIVYRGKCNSTDTNNSTSTSDSTVSDTS |
| | VYSTPNLPSTFFTTLDTSTGSQISTTSNTISSTTNTLAASSITTLNTSTNT |
| | TSYTASTLTALSSTHINSPILSNNATLYTTSLDNTTTDITSSESSINASTI |
| | HNTTYSPVTSIAVNCTVAANETNNSSSKNCQQDIGTIRVKSTTLTAEEGKN |
| | ITIQGNSTWNCPNVVWYRHYNWSTHGHHIYPNRRYQTPTYQHKILTSHPIC |
| | HPDVSSPAAYHDLCRSCNKTELRIYDLNTTNSGRYSRRCYKEYNHDGPHED |
| | ENFGLTVNPRNDTDNHTTPLCPRYVGTQSEEDEDDDYTLSTITNNNMRKTS |
| | HRDISHGTRTTWALTLICIACILLFFVRRALNKRYRPLRDDISESSFVVRY |
| | HPEHED (SEQ ID NO: 15) |
| Soluble gp34 | SSSNAVEPTKKPLKLANYRATCEDRTRTLVTRLNTSHHSVVWQRYDIYSRY |
| C150S | MRRMPPLCIITDAYKETTRQGGAAFACTRQNLTLYNLTVKDTGVYLLQDQY |
| | TGDVEAFYLIIHPRSFCRALETRRSFYPGPGRVVVTDSQEADRAIISDLKR |
| | QWSGLS (SEQ ID NO: 16) |

Host Fc receptors plasmids, CD16A V158, CD16A F158, FcRn-GST, CD16-GST were a kind gift from the George Georgiou Lab. These plasmids were used to transfect Expi293 and purify the individual receptors using IMAC Sepharose resin (Cytiva). Wash buffer consisted of PBS and elution was performed using 100 mM EDTA+PBS. The GST labeled receptors were purified using GSTrap columns (Cytivia) on the AKTA Pure FPLC (GE Healthcare). Elution was performed using 10 mM reduced glutathione (Tocris). All proteins were buffer exchanged into PBS using Amicon Ultra-10 centrifugal spin columns (Millipore).

Antibody expression and purification. Full-length antibody versions of all Fc variants were cloned as previously described (Nguyen et al., 2018) into the following Fab backbones: humanized 4D5, SM5-1 (Liu et al., 2021), hu2B1 (discovered in Maynard Lab), and mouse 27-287 (Schoppel et al., 1996). Antibodies were expressed in ExpiCHO (ThermoFisher Scientific) cells according to the high titer protocol and purified on a Protein A HiTrap column (GE Healthcare) with the AKTA Pure FPLC system (GE Healthcare). Column was washed using 25 mM Tris-HCl and 25 mM NaCl, pH 7.2. Antibodies were eluted using 100 mM sodium acetate at pH 3.0. All antibodies were then buffer exchanged into PBS using Amicon Ultra-30 centrifugal spin columns (Millipore). Each purified antibody was loaded at 3 μgs per well in 4-20% SDS-PAGE (Bio-Rad) gels under reducing and non-reducing conditions and also analyzed by analytical size exclusion chromatography on a Superdex S200 column (GE Healthcare). The human Fab domains of hu2B1 and SM5-1 was additionally cloned into the mouse IgG2A Fc domain in a pCDNA3.0 vector. Protein production and purification was followed as previously described using ExpiCHO cells and protein A.

Viral Fc receptor expression and staining on cell surface. Extracellular ectodomains of gp68 (aa #26-289), gp34 (aa #24-182), Merlin RL13 ANT (Uniprot: Q6SWC9; aa #100-246 (Cortese et al., 2012)), AD169 RL12 (Uniprot: B8YE37; aa #32-369), Merlin RL12 (Uniprot: Q6SWD0; aa #39-366) were cloned into the pcDNA3.0 vector with a murine IgK signal sequence, c-term FLAG (M2) tag, and the PDGFRα transmembrane domain. Plasmids were transfected in 2 mLs of ExpiCHO cells using manufacturer's protocol and were allowed to express for 48 hours at 37° C./8% CO₂ in a 6 well plate (Corning). Cells were washed with wash buffer (1% FBS+PBS) and seeded at 100,000 per well in a 96-well v-bottom plate (Corning) prior to staining. Antibody variants at varying concentrations (1000, 500, 250, 125, 62.5, 31.25, 15.625, 0 nM) were used to stain cells in duplicates on ice for one hour. Cells were then washed 3 times in wash buffer prior to addition of anti-FLAG (M2)-PE (Biolegend, 637309) and goat-anti-human Fcγ-AF647 (Jackson Immuno-Research, 109-606-170) secondaries at a 1:500 dilution. Staining was done on ice for one hour prior to washing 3 times and reading samples on the Fortessa using the HTS plate reader. Percent positive of both PE and AF647 were then gated out and the GMFI of the gated population was reported. No antibody well's GMFI was subtracted from the experimental values.

Library construction, yeast display, and FACS screening. Human IgG1 (aa #233-437) was cloned into the pCTcon2 (Chao et al., 2006) (gift from Georgiou Lab) using a forward primer with an NheI cut site (5′ GGTGGTTCTGCTAGCGACAAAACTCAC 3′; SEQ ID NO: 17) and reverse primer with an XhoI cut site (5′ TTATCAGATCTCGAGCTATTATTTACCCGGAGACAGGG 3′; SEQ ID NO: 18) primer. The PCR was generated using Q5 DNA Polymerase (NEB) and digested using XhoI and NheI (NEB), alongside the pCTcon2 vector. The two pieces were then ligated using T4 ligase (NEB) and transformed DH5a (NEB) electrocompetent cells and plated on 2XYT-amp plates (46 g/L 2XYT (Fischer), 18 g/L agar, 100 μg/mL of ampicillin (Sigma)). Positive colonies were miniprepped (Qiagen) and then transformed into EBY100 (ATCC MYA-494, (Boder and Wittrup, 2000)) chemically competent cells using the Frozen-EZ Yeast Transformation II Kit (Zymo Research T2001). Transformations were plated on selective media plates (6.7 g/L Yeast Nitrogenous Base plus ammonium sulfate (YNB, Fischer), 20 g/L casamino acids (CAA, Fischer) 2% glucose, 100 U/mL pen-strep, 18 g/L agar (Fischer)) and allowed to recover at 30° C. for 2 days. Yeast colonies were verified for sequencing using colony PCR followed by Sanger Sequencing. Library PCRs were first generated with shorter primers on the CH₂ domain of the Fc sequence (aa #233-340), forward (5′ GGTGGTTCTGCTAGCGACAAAACTCAC 3′; SEQ ID NO: 19) and reverse (5′ GGTGTACACCTGTGGTTCTCGGGGCTGCCC 3′; SEQ ID NO: 20). Error prone PCR was done using Taq DNA polymerase with different ratios of buffers and dNTPs (Cirino et al., 2003). Longer primers were used to amplify the PCR product and introduce at least 40 base pairs overhangs. The pcTCon2 vector was digested using XmaI (NEB) and PstI (NEB). Fresh EBY100 electrocompetent cells were generated and 200 Ls was added to 2 mm electroporation cuvettes (BioRad) mixed with 2 gs of vector and 4 gs of PCR insert (Boder and Wittrup, 2000). Five transformations were generated using Gene Pulser (BioRad) per library and allowed to recover in YPD medium prior to spinning down and resuspending in 100 mLs of selective medium (YNB, CAA, pen-strep, 2% glucose). Libraries were grown at 30° C. overnight shaking at 250 rpm and were further passaged into induction medium ((YNB, CAA, pen-strep, 2% galactose) at 20° C. Library size was determined by diluting recovered transformations onto selective media agar plates. A final error rate of 1% was generated for both libraries verified by colony PCR and Sanger Sequencing of 10 individual yeast colonies.

For staining the library, monomeric biotinylated CD16 V158 (Sino Biological) and biotinylated FcRn (Acro Biosystems) were made into tetramers using streptavidin-AF647 (Jackson ImmunoResearch, 016600084) or streptavidin-PE (BioLegend, 405204).

For sorting loss of gp34 binding, the library was passaged into selective media and then induced, as previously mentioned, prior to staining. For the first round of sorting, gp34 library (1E7 cells) were centrifuged at 1000×g, 5 min and stained in 1 mL of 10 nM of CD16 V158 tetramer (AF647) in sterile wash buffer (1% BSA+PBS) for one hour on ice. It was then washed 3× in wash buffer and sorting for CD16A binding clones (1% of population) was performed on the FACS FusionAria. At least 1E5 cells were collected in selective media and allowed to grow up at 30° C. before inducing. Further rounds of sorting included 10 nM CD16A V158 tetramer (AF647) in the presence of unlabeled 5 μM gp34-M, and at least 0.5-1% of the population was collected. Sorting in the presence of soluble gp34-M was performed 3 times in total.

For sorting loss of gp68 binding, the library was passaged in selective media and induced, as previously mentioned, prior to staining. For the first round of sorting, gp68 library (1E7 cells) stained in 1 mL of 10 nM of CD16 V158 tetramer (AF647) and 10 nM of FcRn tetramer (PE) in sterile wash buffer at pH 6.0 (1% BSA+PBS) for one hour on ice. It was then washed 3× in wash buffer (pH 6.0) and sorting for CD16A/FcRn binding clones (1% of population) was performed on the FACS Fusion Aria. At least 1E5 cells were collected in selective media and allowed to grow up at 30° C. before inducing. Further rounds of sorting included 10 nM CD16A V158 tetramer (AF647) and 10 nM of FcRn tetramer (PE) in the presence of unlabeled 10 μM t-gp68, and at least 0.5-1% of the population was collected. Sorting in the presence of soluble t-gp68 was performed 2 times in total. In the final round, 5 μM of gp34-M was added alongside t-gp68, FcRn, and CD16A to ensure no recovery of gp34 binding was introduced.

Rounds from libraries were plated on selective media plates and allowed to grow for 2 days in 30° C. incubator. Ten colonies per round were subjected to colony PCR. Fragments were purified using Zymo Gel and DNA Recovery Kit and submitted to Sanger Sequencing. Unique yeast clones were grown up and induced and characterized for binding to FcRn (pH 6.0), CD16A, gp34-M, and t-gp68 using BD Fortessa. Clones that maintained binding to FcRn (pH 6.0), CD16A, and lost binding to gp34-M (library 1) or t-gp68 & gp34-M (library 2) were then cloned into full length antibodies.

Differential scanning fluorimetry. The thermal unfolding temperatures of the antibodies (0.250 mg/ml) were assessed in duplicate using the Protein Thermal Shift Dye Kit (ThermoFisher Scientific) according to the kit instructions. Continuous fluorescence measurements (λₑₓ=580 nm, λₑₘ=623 nm) were performed using a ThermoFisher ViiA 7 Real-Time PCR System, with a temperature ramp rate of 0.05° C./sec increasing from 25° C. to 99° C.

ELISA. Competition ELISA with recombinant protein that included FLAG tag (CD16A GST, FcRn GST, and gp34-M). Unlabeled competitors (gp34-M, t-gp68) were diluted down the plate by 5-fold and incubated with labeled proteins (2-5 μg/mL). Competition was analyzed as the knockdown of labeled protein using an anti-FLAG (M2) HRP antibody (Sigma A-8592). FcRn-GST binding was performed at pH 6.0, whereas the rest of the assays were performed in 5% milk PBST at pH 7.4.

Two methods were performed for ELISAs, either receptor-coated or antibody-coated. For receptor binding ELISAs, soluble receptors were coated for one hour at room temperature at 4 μg/mL (t-gp68, gp34-M, CD16A-GST, or FcRn-GST (pH 6.0)). Human IgG1 antibodies (Hu4D5 Fab arms with the WT, M428L/N434S aka LS, N434S, N434Y, or M252Y/S254T/T256E aka YTE changes in the Fc) were diluted down 5 fold (starting at 25 μg/mL) in the presence of 5% milk PBST and binding was detected using an anti-kappa HRP antibody (Southern Biotech 2060-05). For antibody binding ELISAs, antibodies (hu4D5 WT, 3S4, R47, G8, G2, G5, YTE) were coated for one hour at room temperature at 4 μg/mL and then blocked with 5% milk PBST. Labeled receptors, t-gp68, gp34-M, CD16A-GST, or FcRn-GST (pH 6.0 or 7.4) were diluted down 5 fold (starting at 450 nM) in the presence of 5% milk PBST and binding was detected using an anti-FLAG-M2-HRP antibody.

All curves were fit with 4PL curve using GraphPad.

gB-coated beads, labeled-AD169 prep, and ADCP assay. Red fluorescent polystyrene beads (Bangs Laboratory, FSDG004) were washed with PBS three times in Centrigual Filter (Millipore) and then resuspended in 100 μg/mL of gB protein purified from Expi293. The beads were incubated in the dark for 1 hr, rotating. Excess gB was removed by spinning down filter unit at 1000×g and resuspending in 5% FBS+PBS conjugated with 1:100 pHrodo Green iFL STP ester (Thermo Fischer Scientific, P36013). Excess phrodo was removed by spinning down filter unit at 1000×g and beads were resuspending in 5% FBS PBS. Final concentration of beads was 5E8 beads/mL.

Approximately 3E6 AD169 virions were thawed from −80° C. and were buffer exchanged in PBS using Amicon Ultra-10 centrifugal spin columns (Millipore) and concentrated to final volume of 100 μLs. Five microliters of reconstituted phrodo iFL Red STP Ester (Thermo Scientific) was then added and allowed to incubate at room temperature for 1 hour. Labelled virions were subsequently buffer exchanged into PBS to remove excess dye and diluted to 4E5 PFU/mL in complete medium. Fifty microliters of diluted virus (2 PFU/cell) was added to 100 μL of THP-1 (5E6 cells/well) in the presence of complete media (RPMI+1% FBS) in a 96-well U-bottom tissue culture treated plate (Corning). For antigen coated beads, the same set up was performed but in the presence of diluted beads at a final concentration of 1:50 cell to bead ratio. Fifty microliters of serially diluted antibodies in complete media (RPMI+1% FBS) were added to wells and allowed to incubate for 4 hours at 37° C./5% CO₂. The plate was kept on ice and washed 3× in cold 5% FBS+PBS. Cells were analyzed using PE, AF647, and FITC channels on the Fortessa HTS plate reader. For virion ADCP assay, phagocytosis was measured as the percent pHrodo+(PE) multiplied by the GMFI of PE population. For beads ADCP assay, phagocytosis was measured as the percent bead+(AF647)/pHrodo+(FITC) multiplied by the GMFI of AF647 population. No antibody wells were subtracted from the results.

SKOV3 ADCC. ADCC assays were performed on SKOV3 cells using the NK-92 (F158 or V158). SKOV3 cells were labeled in serum-free medium with 2 mM calcein-AM (BD Pharmingen) for 30 min in the dark at 37° C./5% CO₂. SKOV3 labeled cells were spun down and resuspended 3 times in complete media (DMEM+10% FBS). NK-92 cells were also spun down and resuspended in complete media at 2E6 cells/mL. SKOV3 cells were seeded at 10,000 cells/well (100 μL of 1E6 cells/mL) in a 96 U bottom tissue culture treated plate (Corning) and 50 μL of antibody diluted in complete media was added (10, 1, and 0.1 μg/mL final concentrations). Fifty microliters of NK-92 cells were added as well at a final E:T ratio of 10:1. ADCC incubation was done at 37° C./5% CO₂ for 4 hours. Cells were then spun down at 300×g and 100 μL of supernatant was transferred to clear-bottom black 96 well plate (Corning). Emission and excitation (480/525) was collected using a plate reader. Percent ADCC was measured: (Experimental−Spontaneous Release)*100/(Maximum lysis−Experimental). Maximum lysis is SKOV3 cells lysed with RIPA buffer (Thermo Scientific) and spontaneous release was measurement of SKOV3 cells with no antibody or NK-92 cells.

BW reporter assay. This reporter assay is based on BW5147 reporter cells stably expressing human CD16A as a chimeric molecule providing the ectodomain of the FcγR fused to the transmembrane domain and cytosolic tail of mouse CD3 (Corrales-Aguilar et al., 2013). Briefly, MRC5 fibroblasts were infected at MOI of 2-5 (or as indicated) with BAC-derived HCMV AD169 WT or respective vFcγRs mutants (Δgp34Δgp68Δgp95) for the indicated time points before being used as target cells in the reporter cell assay. Next, target cells were pre-incubated with titrated amounts of antibody as indicated in medium (RPMI) supplemented with 10% FCS for 30 min at 37° C., 5% CO₂. After opsonization, target cells were washed with PBS/10% FCS and co-cultured with BW5147-reporter cells (ratio E:T 20:1) expressing host FcγRIII ectodomain for 16 h (overnight) at 37° C. in a 5% CO₂ atmosphere. Reporter cell mIL-2 secretion was quantified by subsequent anti mIL-2 sandwich ELISA as described previously (Corrales-Aguilar et al., 2013).

CD107a NK cell degranulation assay with HCMV infected cells. NK-92 V158 or F158 were resuspended at 2E6 cells/mL in the presence of 6 μg/mL Golgi Stop (Monesin, BD Pharmingen) and 10 μg/mL Golgi Plug (Brefeldin A, BD Pharmigen) and anti-LAMP1-APC antibody at 1:50 dilution (Biolegend). HCMV infected cells (MOI=2, 96 h) were detached from plates using 10 mM EDTA and washed and resuspended 3× with complete media and then seeded onto 96 well U bottom tissue culture treated plates (Corning) at 1E5 cells per well (viability>90%). SM5-1 Fc variants were diluted in complete medium and added to the HCMV infected wells. Finally, NK-92 cells were added to the wells and allowed to incubate at 37° C./5% CO₂ for four hours. Controls included non-incubated NK-92 cells. Cells were spun down at 300×g and washed 3× with cold 1% FBS+PBS. Cells were resuspended in cold PBS and were analyzed using the Fortessa HTS reader. Flowjo software was used to obtain APC signals from samples and no antibody control was subtracted from each sample. Experiments were performed in duplicates with technical replicates in each set.

Internalization assay followed by cell staining. Antibodies were labeled with phrodo iFL Red STP Ester (Thermo Scientific) according to manufacturer's protocol. They were then buffer exchanged into PBS using the Superdex S200 column (GE Healthcare) on AKTA Pure FPLC (GE Healthcare) and concentrated using Amicon Ultra-30 (Millipore). HCMV infected cells (MOI=2, 96 hrs) were detached from plates using 10 mM EDTA and washed and resuspended 3× with complete media (DMEM, 10% FBS) and then seeded onto 96 well U bottom tissue culture treated plates (Corning) at 1E5 cells per well (viability>90%). Antibodies were diluted in complete media and allowed to incubate with HCMV infected cells for 2 hours at 37° C./5% CO₂. Cells were then washed 3 times in wash buffer prior to addition of goat-anti-human Fcγ-AF647 (Jackson Immuno-Research, 109-606-170) secondary at a 1:500 dilution. Staining was done on ice for one hour prior to washing 3 times and reading samples on the Fortessa using the HTS plate reader. Flowjo software was used to obtain AF647 and FITC signals from samples and stained-no antibody control was subtracted from each sample. Experiments were performed in duplicates with technical replicates in each set.

K_{D} determination using surface plasmon resonance (SPR). SPR was performed using a Biacore X100. Two methods were used to determine kinetics for gp68. The first method involved immobilizing Fc2 of a CM5 chip (Cytiva) with 200 RU of human IgG1 Fc using 10 mM sodium acetate at pH 4.0. Fc1 remained as reference channel. Receptor, t-gp68-strep-FLAG, was injected at variable concentrations (250 to 3 nM) with 2-fold dilutions. Blank injections (0 nM) were used to subtract curves. Association time was 180 seconds, while dissociation was 300 seconds. Regeneration was performed using 10 mM glycine at pH 1.5, while 0.5 M arginine was used as an additional wash step post-regeneration. All analysis was performed using BiaEvaluation X100 software using 1:1 binding kinetics.

The second method included immobilizing Fc2 and Fc1 of CM5 chip with 4500 RU of anti-strep Fab (produced by Professor Jason McLellan's Lab at UT Austin, clone C23.21, patent #WO2015067768). Receptors (t-gp68-twin strep-FLAG and gp34-M strep-FLAG) were injected through Fc2 only at a final capture response of 40 RU. Different concentrations of hu4D5 IgG1 Fc variants were injected with an association time of 180 seconds, while dissociation was 300 seconds. Regeneration was performed using 10 mM glycine at pH 1.5, while 0.5 M arginine was used as an additional wash step post-regeneration. All analysis was performed using BiaEvaluation X100 software. For gp34 binding, 1:1 binding kinetics were performed. For t-gp68, 2:1 binding kinetics were performed as well as steady state kinetics.

K_{D} determination using biolayer interferometry. Equilibrium dissociation constants (K_{D}) of engineered Fc variants to FcRn and FcγRs were determined by steady-state analysis on an Octet® RED96e instrument. For binding measurements of FcγRs, hu4D5 IgG1 antibody variants were immobilized onto CH1-binding (FAB2G) biosensors (Sartorius Cat. #18-5125) to a response level of 3 nm. Antibody-coated sensor tips were dipped into two-fold serial dilutions of CD16A F158 and CD16A V158 at concentrations of 5000-156.25 nM and 2000 nM-62.5 nM, respectively. Loading, baseline, association, and dissociation steps were performed at room temperature in PBS, pH 7.4, with 0.02% Tween-20 and 0.1% BSA. Sensor tips were regenerated using 10 mM glycine, pH 1.7. For measurements of FcRn K_{D}, biotinylated FcRn (AcroBiosystems Cat. #FCM-HB2W4) was immobilized onto streptavidin (SA) biosensors (Sartorius Cat. #18-5019) to a response level of 0.5 nm. FcRn-coated sensor tips were dipped into two-fold serial dilutions of hu4D5 Fc variants from 1000-31.25 nM in pH 6.0 and 7.4 PBS with 0.02% Tween-20 at room temperature. Sensor tips were regenerated between cycles using PBS, pH 7.4, with 0.02% Tween-20. All experiments were performed in duplicate. An unloaded tip and 0 nM analyte control were subtracted from response curves prior to analysis of steady-state kinetics using Octet Systems analysis software.

Pharmacokinetics in Tg32 mice. Pharmacokinetic studies were performed in transgenic hFcRn Tg32 mice (The Jackson Laboratory Cat #014565). Mice were administered 2 mg/kg of 4D5 antibody Fc variants at 5-6 weeks of age by intraperitoneal injection. Blood from the lateral tail vein was collected every 3-4 days. Serum concentration of antibody was determined by ELISA as follows: High-binding 96-well plates (Corning Cat #9018) were coated with 0.5 μg/mL of chimeric Her2-Fc (R&D Systems Cat #1129-ER) antigen. Plates were blocked with 5% milk in PBS with 0.1% Tween and incubated with diluted serum samples (1:400-1:25 depending on the time point) or known concentrations of 4D5 antibody diluted with 1:100 un-injected mouse serum in duplicate. 4D5 antibody was detected by incubation with goat anti-human kappa light chain antibody-HRP (Southern Biotech Cat #2060-05, 1:2000 dilution). Absorbance at 450 nm was measured after application of TMB substrate (Thermo Scientific Cat #34021) and neutralization with 1 M HCl. Standard curves for 4D5 antibody were generated for each ELISA plate. A four-parameter fit for each standard curve was generated in Prism (GraphPad Software) and used to quantify 4D5 antibody present in serum samples. The beta-phase elimination constant (Kₑ) was determined by log-linear regression of the concentration data, including at least three time points with measurable concentrations. Beta-phase half-life was determined as follows:

$${T}_{β 1 / 2} = 0.6 \; 93 / {K}_{e}$$

AUC_{inf} (area under the curve to infinity) was calculated using the trapezoidal rule up to the last measurable time point. Clearance (CL) was calculated with the equation:

$$CL = Dose / {AUC}_{inf}$$

AUMC_{inf} (area under the moment curve to infinity) was calculated using the trapezoidal rule up to the last measurable time point. Steady state volume of distribution (Vₛₛ) was calculated with the following formula:

$${V}_{ss} = * \frac{CL ⋆ {AUMC}_{inf}}{{AUC}_{inf}}$$

Negative stain electron microscopy. Purified complexes of human IgG Fc bound to gp34-M and t-gp68 or gp34-M only were diluted to a concentration of 0.02-0.03 mg/mL using 2 mM Tris, pH 8.0, 200 mM NaCl, and 0.02% NaN₃. Sample dilutions were performed immediately before depositing on plasma cleaned CF-400 grids (EMS) and stained using uranyl acetate (neutralized to pH 7). Grids were imaged at a nominal magnification of 60,000× (corresponding to a calibrated pixel size of 3.6 Å/pix) in a JEOL 2010F TEM microscope equipped with a Gatan OneView Camera. CTF estimation and particle picking were performed in cisTEM (Grant et al., 2018) and extracted particles were exported to cryoSPARC v2 (Punjani et al., 2017) for 2D classification and ab initio 3D reconstruction and heterogenous refinement.

Western blots. Human MRC5 cells were infected with 5 PFU/cell of HCMV AD169 WT or respective vFcγRs mutant (Δgp34Δgp68Δgp95). Seventy-two hours post infection, cells were washed once with PBS and lysed using NP40-containing buffer (140 mM NaCl, 5 mM MgCl₂, 20 mM TRIS, pH 7.6 and 1% NP40). Cell debris was sedimented at 13000 rpm, 20 min, 4° C. A sample of each cleared lysate (as indicated) was taken for subsequent western blot expression analysis. Proteins were separated by 10%-sodium-dodecyl-sulfate (SDS)-polyacrylamide gel electrophoresis (PAGE) and transferred to nitrocellulose membrane using iBlot 2 Dry Blotting System (Thermo Fisher Scientific). Western Blot was performed with anti-IE1-specific mouse mAb (1/1000) (ARGENE, #11-003) or mouse anti-gB (SM51 WT huIgG1, 1 μg/mL), following and anti-mouse-peroxidase (Dianova, Germany). Proteins were visualized using ECL chemiluminescence system (Li-Cor Odyssey, Germany). Membranes were re-blotted staining for b-actin as loading control (mouse anti-β-actin, Sigma #A2228).

Cell staining of HCMV infected cells (FACS). Human MRC5 cells were infected with an MOI 5 of WT HCMV or HCMV vFcγR mutant (ΔΔΔ). Seventy-two hours post infection, cells were harvested using Accutase (Sigma-Aldrich) to retain surface molecules upon detachment from 6-well plates. Harvested cells were washed twice in PBS/3% FBS and centrifuged at 1000×g and 4° C. for 3 min. Cells were then incubated with staining buffer containing either mouse anti-gB (SM51) (1/70), mouse anti-gB-A647 (1/70), anti-gH-MS-109 (1/20), Cytotect (1/50) or human Fc-fragment-TexasRed (1/20) or no antibody. Unconjugated primary antibody was stained using secondary antibody as indicated goat-anti-mouse IgG A647 (1/500) or goat-anti-human IgG APC (Fab2, 1/100). Further incubation steps were carried out at 4° C. for 1 h and followed by three washing steps in staining buffer. Analysis was performed on a FACS Fortessa instrument (BD Bioscience) and data were analyzed with FLowJo (Tree Star Inc, USA).

Static light scattering. Prior to static light scattering (SLS) measurements, samples were run on size exclusion chromatography (S200) and correct sizes were isolated and concentrated using Amicon Ultra-10 centrifugal spin columns (Millipore) to at least 10 mg/mL.

SLS measurements as a function of concentration were performed at room temperature with a laser wavelength A=658 nm and a scattering angle of 900 using the miniDAWN TREOS from Wyatt Technology (Santa Barbara, CA) run in batch mode with the microcuvette accessory at ambient temperature. The scattering intensity was allowed to stabilize at equilibrium value and then recorded and averaged over a period of ˜1 min. The data was collected and processed using the Astra 6.1.2 software (Wyatt Technology, Santa Barbara, CA).

The molecular weight can be determined from scattering measurements in the dilute limit using the equation:

$$\frac{Kc}{{R}_{θ}} = \frac{1}{{M}_{w}} + 2 {B}_{22} c$$

Where R_{θ} is the excess Rayleigh ratio calculated from scattering intensity, M_{w} is the molecular weight, B₂₂ is the second virial coefficient, and K is an optical constant given by:

$$K = \frac{4 {π}^{2} {n}^{2}}{{N}_{A} {λ}^{4}} {( \frac{dn}{dc} )}^{2}$$

Here, N_{A} is Avogadro's number, dn/dc is the refractive index increment due to protein molecules, assumed for simplicity to remain constant at 0.185 mL/g (Minton, 2007).

$$n = {n}_{0} + ( \frac{dn}{dc} ) {c}_{p r o t}$$

is the solution refractive index (RI) calculated using the solvent RI (n₀=1.331 for PBS) and dn/dc.

Binding studies: BLI and SPR were conducted with at least duplicate measurements and presented as the mean±SEM of the inducated number of replicates. Details can be found in figure legends and detailed methods section.

Statistical analyses: Software used for statistical analyses described were done using GraphPad Prism (v8.0.2.). Additional software for analysis include FlowJo software (Treestar Inc.) for flow cytometry. Details can be found in figure legends for statistical models used.

Flow cytometry to detect binding of Fc variants to HSV-1 gE/gI. Binding of Fc mutants to HSV-1 gE/gI was evaluated in a flow cytometry assay. Antibodies with a 4D5 (Herceptin) binding region and WT or mutated huIgG1 Fcs were cloned into mammalian expression plasmids and solubly expressed in ExpiCHO cells. They were then purified using Protein A affinity chromatography and SEC. HSV-1 gE/gI from the KOS strain was expressed on the surface of ExpiCHO cells using a bicistronic mammalian expression plasmid containing the following: a signal sequence, gE ectodomain and transmembrane domains, furin protease site, t2a peptide, gI ectodomain and transmembrane domains. ExpiCHO cells were transfected using the HSV-1 gE/gI construct and cultured for two days at 37° C./8% CO. Successful expression of gE/gI on the surface was detected by staining transfected and untransfected ExpiCHO cells with 5 ug/mL anti-gE antibody (Visusys Catalogue #H1A054-100) for 1 hour on ice, followed by staining with a 1:500 dilution of anti-mouse Fcγ AF647 antibody for 1 hour on ice (Jackson ImmunoResearch Catalogue #115-605-071). Binding of the mutated Fcs was detected by staining gE/gI-transfected and untransfected cells with each antibody at 50 ug/mL for 1 hour on ice followed by staining with a 1:500 dilution of anti-human Fcγ AlexaFluor 647 (Jackson ImmunoResearch Catalogue #109-605-098) for 1 hour on ice. All staining was done in pH 7.2 PBS with 5% FBS. Fluorescence was detected using a BD Fortessa system.

### Example 2—vFcγR Deletion Enhances CD16A Activation while Reducing Anti-gB Internalization

The inventors first aimed to understand the impact of vFcγRs on inhibition of CD16A activation and antibody internalization. For these studies, the gB-specific antibody SM5-1 (Liu et al., 2021) was used, in addition to the polyclonal antibody Cytotect used in prior studies (Kolb et al., 2021, Corrales-Aguilar et al., 2014b). SM5-1 binds pre- and post-fusion conformations of the gB fusogen on infected cells and virions, but mediates minimal NK cell activation against HCMV-infected cells in vitro (Nelson et al., 2018). The synchronized gB and vFcγR expression kinetics result in peak cell surface levels 72-96 hours post-infection (Brey et al., 2018, Proff et al., 2018). Accordingly, human MRC5 fibroblasts were infected with strain AD169 or an isogenic variant lacking expression of all four vFcγRs (ΔΔΔ) at an MOI=5 and used in assays 72-96 hours later. Similar levels of gB expression for both HCMV strains were seen by flow cytometry and western blotting when using SM5-1 with a mouse IgG2a Fc (SM5-1-mFc; FIG. 6A-B). This isotype does not bind HCMV vFcγRs (MacCormac and Grundy, 1996).

Infected cells were co-cultured with mouse BW-CD16A-ζ effector cells that stably express the human CD16A ectodomain fused to mouse CD3ζ transmembrane and intracellular domains (Corrales-Aguilar et al., 2013). CD16A activation was monitored by mouse IL-2 secretion, which was quantified by ELISA. Significantly greater CD16A activation was observed when Cytotect was incubated with ΔΔΔ versus AD169-infected cells (p<0.0001; FIGS. 1A,6C). Similarly, greater CD16A activation was observed for cells infected with ΔΔΔ versus AD169 when SM5-1 with a human IgG1 Fc was added (SM5-1-huFc; p<0.0001; FIGS. 1A,5D).

To visualize antibody binding to and internalization into HCMV-infected cells, fluorescently labelled SM5-1 was incubated with human or mouse Fc at 20 μg/mL with AD169/GFP infected fibroblasts for 2 h. SM5-1-huFc showed high levels of cellular internalization as compared to SM5-1-mFc, which primarily stained the cell membrane of GFP-positive cells (FIG. 1B). To quantify differences in antibody internalization by infected cells, SM5-1-huFc and a human IgG1 isotype control were labeled with the pH-sensitive pHrodo dye and incubated at 67 nM with AD169- and ΔΔΔ-infected fibroblasts for 2 h prior to detecting extracellular antibody with a fluorescent anti-huFc. SM5-1-huFc stained both AD169- and ΔΔΔ-infected cells (˜80%) but not mock infected cells (5%, p<0.0001). A huFc control antibody bound cells infected with AD169 (˜35%), which was significantly reduced for ΔΔΔ- and mock-infected cells (˜5%; p<0.001; FIG. 1C). SM5-1-huFc was internalized to a much greater extent when incubated with AD169- versus ΔΔΔ-infected cells (50% vs 7%, p<0.0001) and when compared to the huFc control (10% AD169-infected cells, p<0.0001; FIG. 1D), indicating that specific internalization of anti-gB antibodies is mediated by vFcγRs. Collectively, these data led to the hypothesis that disrupting Fc:vFcγR interactions could improve the anti-viral activities of antibodies targeting immunogenic HCMV antigens, such as gB.

### Example 3—Engineering Soluble Gp34 and Gp68 Ectodomains

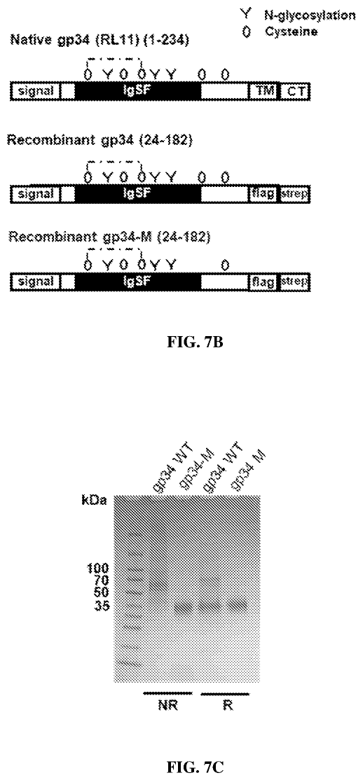

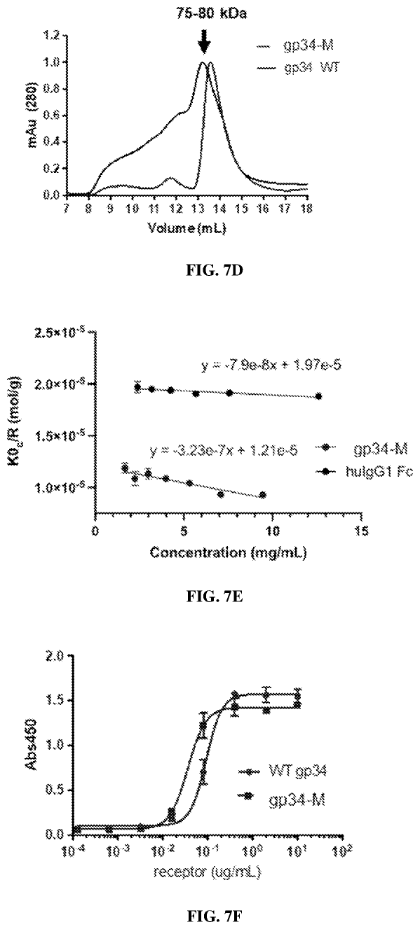

gp68 (t-gp68) was truncated to remove the heavily 0-glycosylated N-terminus, but retain the predicted immunoglobulin domain between residues 69-289, and appended with C-terminal FLAG and Strep-Tags®. CHO cell expression followed by strep tag purification yielded monodisperse protein with an apparent molecular weight of ˜60 kDa (FIG. 7A) (Sprague et al., 2008). Prior efforts to characterize the gp34 ectodomain (residues 24-182) (FIG. 7B) were hindered by protein aggregation (Sprague et al., 2008). Aggregates appearing on non-reducing SDS-PAGE resolved to the expected ˜35 kDa monomer size under reducing conditions, suggesting that cysteine mispairing could be responsible for aggregation (FIG. 7C). The five cysteines in the gp34 ectodomain were individually altered to serine resulting in one variant, gp34-M, with reduced aggregation compared to gp34 on both SDS-PAGE and size exclusion chromatography (SEC; FIGS. 7B-D). Notably, while the apparent gp34-M size on SDS-PAGE is ˜35 kDa under both reducing and non-reducing conditions, measurements by SEC and static light scattering are consistent with formation of a non-cysteine mediated dimer of ˜75-80 kDa under native conditions (FIGS. 7D,E). Additionally, binding of SEC-purified gp34 and gp34-M to human IgG1 by ELISA indicated similar curves (FIG. 7F).

### Example 4—Human and Viral FcγRs Share Overlapping but Distinct Epitopes on Human IgG1

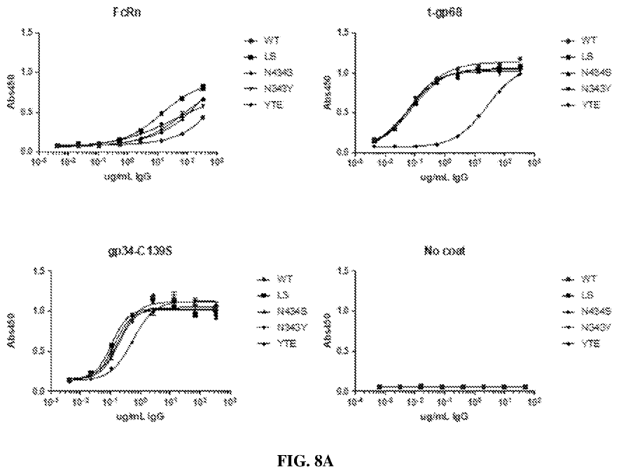

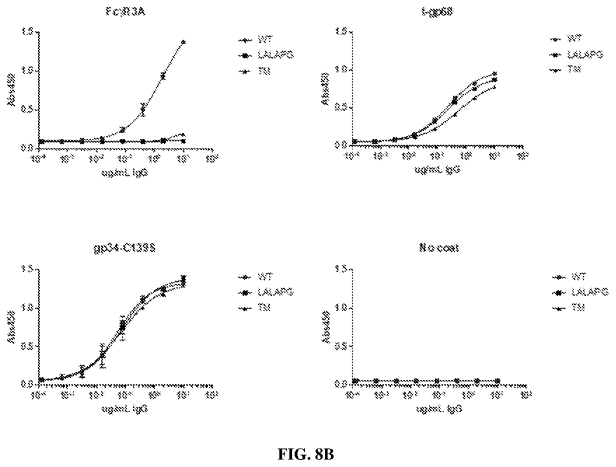

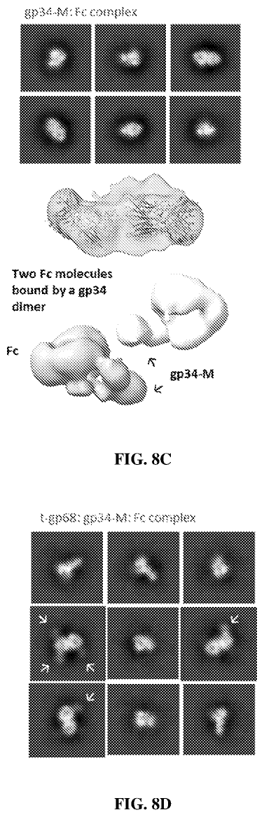

Competition ELISAs were performed to initially localize the vFcγR epitopes on human IgG1 Fc. Using GST-tagged FcRn and CD16A, gp34-M inhibition of CD16A and t-gp68 inhibition of FcRn binding to immobilized human Fc was observed (FIG. 2A). Analysis of gp34-M and t-gp68 binding to four Fc variants with improved FcRn binding (LS, N434S, N434Y, YTE) (Wang et al., 2018) revealed that YTE conversely exhibits reduced binding to gp68 by ELISA (FIG. 8A). Analysis of two Fc variants with reduced CD16A binding, LALAPG and TM, (Wang et al., 2018, Borrok et al., 2017) showed no change in binding to t-gp68 or gp34-M (FIG. 8B). These data indicate that the gp34 and gp68 binding footprints overlap with the CD16A and FcRn footprints but engage different Fc residues, suggesting that Fc variants selectively engaging host but not vFcγRs could be identified.

Negative stain electron microscopy (nsEM) using SEC-purified complexes of huFc with gp34-M and/or t-gp68 (FIGS. 2B-C) allowed us to visualize the relative binding locations of gp34 and gp68. The 3D reconstructions and 2D particle class averages suggest the core complex consists of two Fc molecules bound to a dimeric Fc receptor (FIG. 2D). These dimeric Fc particles were also observed for complexes without t-gp68, suggesting a dimeric gp34-M provides the extra density between Fc molecules (FIGS. 2E,8C). Analysis of particles with Fc and gp34-M suggests that the receptor binds to the upper CH₂ region (FIG. 2E). In the presence of gp34 and t-gp68, some 2D class averages showed appendages extending from the Fcs (FIGS. 2D,8D), which correspond to partially occupied t-gp68 binding sites. Analysis of these and morphologically distinct particles consisting of a single Fc molecule formed with only t-gp68 (FIG. 2F) indicate that t-gp68 binds the CH₂—CH₃ interface. Overall, this analysis revealed 1:2 gp34-M:Fc and 2:1 t-gp68:Fc stoichiometries (FIGS. 2D-F).

The Fc-vFcγR binding kinetics were evaluated with surface plasmon resonance (SPR; Table 1). For t-gp68 binding, human IgG1 Fc was immobilized on a CM5 sensor chip before injecting t-gp68 at multiple concentrations (3-250 nM). This resulted in a K_{D} value of 110±4 nM determined from kinetic constants (FIG. 2G), similar to the previously reported value of 85 nM (Sprague et al., 2008). An anti-strep tag Fab was used to capture gp34-M followed by injecting human IgG1 at multiple concentrations (3-200 nM) to determine a K_{D} value of 7.3±0.2 nM (FIG. 2H). To the inventor's knowledge, no gp34 kinetics have been previously determined. For both vFcγR, the on-rates are modest (˜5×10⁴ M⁻¹s⁻¹), with intermediate (0.004±0.001 s⁻¹; gp68) or slow (0.0004±0.0001 s⁻¹; gp34) off-rates.

TABLE 1
Binding affinities of Fc variants for viral FcγRs measured by SPR.

| | | | | | | | Fold |
|---|---|---|---|---|---|---|---|
| Im- | | | | Kinetic | | Equil- | change: |
| mobilized | | kₒₙ (×10⁵) | k_{off} | K_{D} | | ibrium | K_{D}(variant)/ |
| ligand | Analyte | (M⁻¹, s⁻¹) | (s⁻¹) | (nM) | χ² | K^{D} (nM) | K_{D} (IgG1) |
| IgG1* | gp68 | 0.650 ± | 0.004 ± | 69 ± | 1.1 ± | 80 ± | 1 |
| | | 0.020 | 0.01 | 0.30 | 0.01 | 3 | |
| G2* | gp68 | 0.070 ± | 0.032 ± | 4800 ± | 3.5 ± | 1800 ± | 23-70 |
| | | 0.004 | 0.003 | 700 | 0.06 | 1 | |
| G5* | gp68 | 0.100 ± | 0.021 ± | 2100 ± | 7.2 ± | 850 ± | 11-30 |
| | | 0.008 | 0.002 | 200 | 0.2 | 1 | |
| gp68 | huFc | 0.400 ± | 0.005 ± | 110 ± | 0.5 ± | — | |
| | | 0.010 | 0.001 | 4 | 0.05 | | |
| IgG1 | gp34 | 0.53 ± 0.01 | 0.0004 ± | 7.30 ± | 1 ± | — | 1 |
| | | | 0.0001 | 0.2 | 0.1 | | |
| G2 | gp34 | 0.14 ± 0.01 | 0.004 ± | 320 ± | 0.4 ± | — | 44 |
| | | | 0.01 | 2.0 | 0.1 | | |
| G5 | gp34 | 0.37 ± 0.04 | 0.001 ± | 33 ± | 1 ± | — | 4.5 |
| | | | 0.01 | 0.2 | 0.1 | | |

*For 2:1 binding fits, only the first kinetic constants (kₐ₁, k_{d1})were used to determine KD.
All antibodies expressed with hu4D5 Fab arms. huFc-IgG1 does not include Fab arms.
Dashed lines indicate not measured data.

### Example 5—Soluble Gp34 and Gp68 Inhibit Internalization of Anti-gB Antibodies by HCMV-Infected Cells

To further define vFcγR roles in selective internalization of anti-gB antibodies, soluble gp34-M and t-gp68 were used to competitively inhibit antibody uptake by infected cells. Using the internalization assay above (FIG. 1D), SM5-1-huFc or control antibodies were incubated with infected cells in the presence of soluble gp34-M at 100 nM or t-gp68 at 2000 nM before assessing the fraction of surface-bound and internalized antibody by flow cytometry. The vFcγR concentrations selected are >10-fold above each K_{D} to support effective competition. Minimal binding to AD169-infected fibroblasts was observed for the 2B1 control antibody with mFc and an irrelevant Fab (˜5%), whereas higher binding was observed for a control antibody with huFc and irrelevant Fab (˜40%, reflecting capture by vFcγRs, p<0.0001), and SM5-1-mFc (˜60%, reflecting gB binding, p<0.0001). SM5-1-huFc shows a slight increase in binding due to simultaneous gB and vFcγR capture (˜75%), which is reduced by soluble vFcγRs (˜60%, FIG. 2I). Internalization of SM5-1 and 2B1 control antibodies with huFc was high (37% and 20%, respectively) but greatly diminished (0-5%) in the presence of soluble gp34-M, t-gp68, or both (FIG. 2J). These data provide compelling data implicating gp34 and gp68 in antibody internalization.

### Example 6—Engineering Human IgG1 Fc Variants with Reduced vFcγR Binding

Yeast display has been used to modulate Fc domain affinity for host receptors and stability (Chen et al., 2017). Human IgG1 Fc residues 233-447, spanning the hinge, CH₂ and CH₃ domains, were cloned to the Aga2 c-terminus to present the Fc on the yeast surface in the same orientation as on an opsonized particle (FIG. 3A). After transfection into yeast strain EBY100 (Boder and Wittrup, 2000), the Fc-expressing cells showed high CD16A binding capacity, which was inhibited by soluble gp34-M (FIG. 3B), and high FcRn binding at pH 6.0, which was inhibited by soluble t-gp68 (FIG. 3C), as measured by flow cytometry.

To identify Fc variants with reduced vFcγR but unaltered host FcR binding, error-prone libraries were generated and screened for binding to CD16A in the presence of a large excess of gp34-M competitor. To generate a library spanning the gp34 epitope, the CH₂ domain (233-340) was amplified under error-prone conditions to generate a library of >10⁶ variants with an error rate of 1%, determined from sequencing individual clones. This library was initially sorted to collect variants retaining binding to AF647-labelled CD16A-V158 tetramer, then for CD16A-V158 binding in the presence of excess (1 μM) soluble gp34-M (FIG. 3B). Single yeast colonies were selected from rounds 3 and 4 for sequencing and analysis by flow cytometry, resulting in identification of two variants: one clone containing S337F and another containing H268L, E294K, Q311L, K334E (termed R47; FIG. 9A).

Although Fc variant YTE has reduced affinity for t-gp68 (FIG. 8A), it suffers from poor CD16A affinity and impaired effector functions (Dall'Acqua et al., 2006). To identify a variant with reduced t-gp68 binding that better retains CD16A binding, another ˜1% error-prone library was generated in the CH₂—CH₃ region of variant R47, which showed lower overall gp34-M binding compared to S337F. The library was first sorted for simultaneous binding to tetramerized FcRn and CD16A-V158 at pH 6.0 with subsequent rounds including 5 M soluble t-gp68 as competitor (FIG. 3C). For each round, cells were stained in parallel with FcRn at pH 7.4 with no acquired binding observed. Clones from rounds 3 and 4 were isolated for analysis, identifying a consensus R255Q change in the R47 background (termed G2, FIG. 3D). Variants were generated with only R255Q and combined R255Q with S337F to produce another variant (termed G5) with reduced gp34 and gp68 binding (FIGS. 3D,9E). Interestingly, the S337F and R47 changes that mediate loss to gp34 and the R255Q change which results in reduced gp68 binding occurred near the CD16A and FcRn binding sites, respectively (FIG. 9A).

### Example 7—Selected Fc Variants have Greatly Reduced Binding to Multiple vFcγRs

Three Fc variants (wild-type, G2, G5) were cloned for expression as human IgG1 proteins with multiple antigen specificities: hu4D5, which binds HER2 and SM5-1, binding gB (Liu et al., 2021). Following protein A purification, antibodies were monodisperse as assessed by SDS-PAGE and SEC (FIGS. 9B-C). Binding to host FcR and vFcγR was initially evaluated by ELISA (FIG. 9D), with G2 and G5 showing greatly reduced affinity for t-gp68 and gp34-M, but apparently similar binding to CD16A-GST and FcRn-GST at pH 6.0 and no acquired FcRn binding at pH 7.4 (FIG. 9D). Using SPR, the kinetic and equilibrium K_{D} values were measured for gp34-M and t-gp68, which revealed that G5 lost ˜5 and 10-30-fold affinity, while G2 lost 45 and 23-70-fold affinity, respectively, for each vFcγR (Table 1, FIGS. 3D,9A,9B).

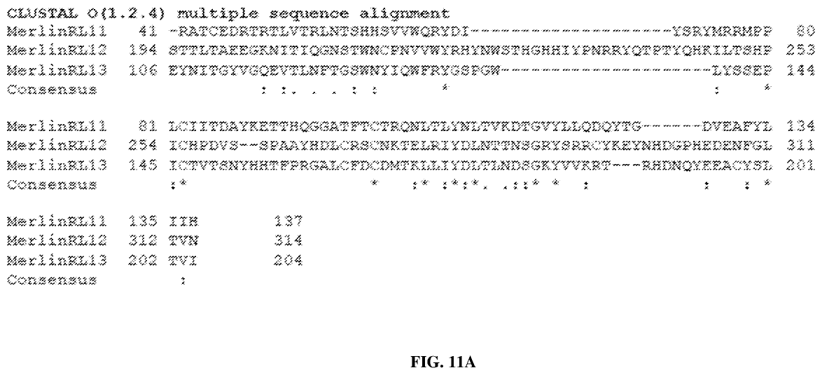

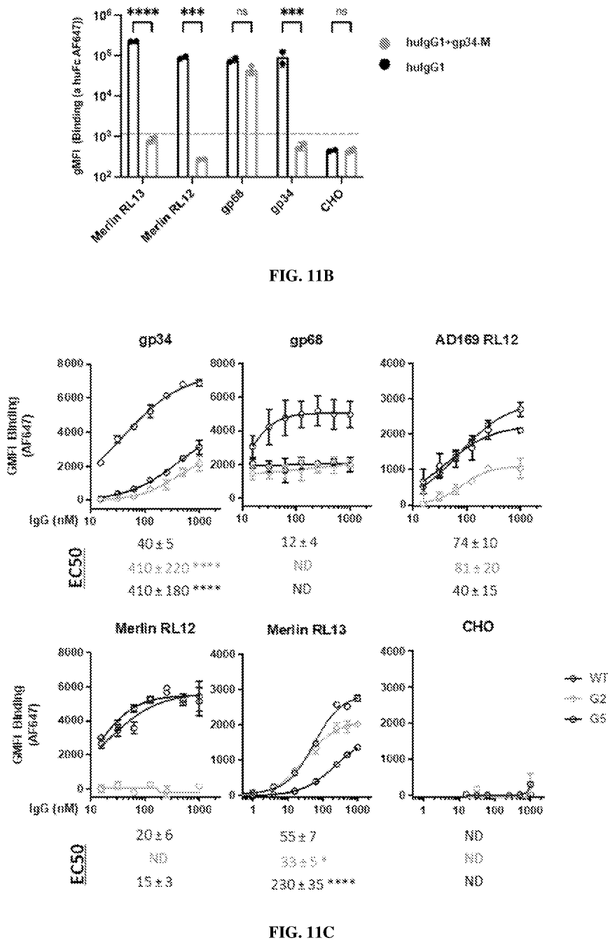

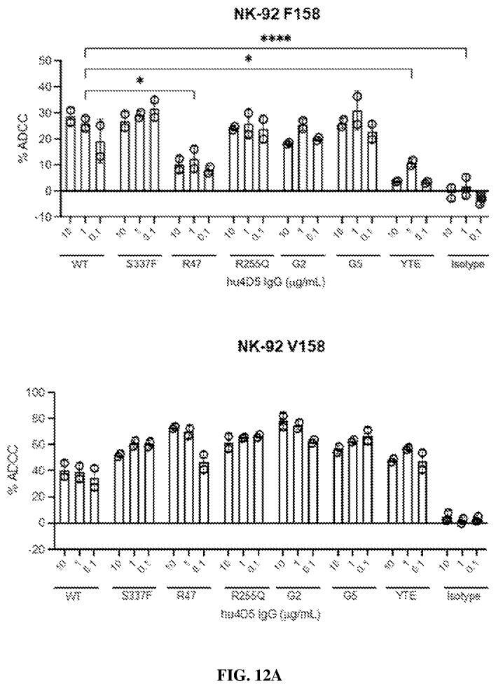

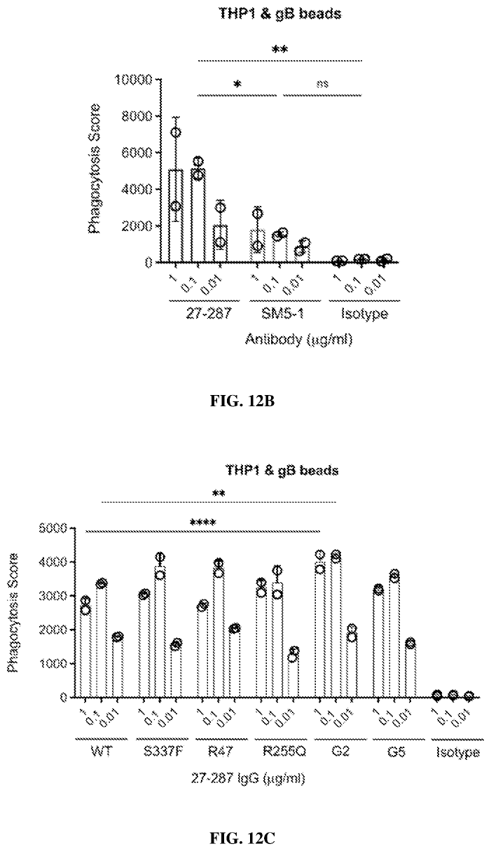

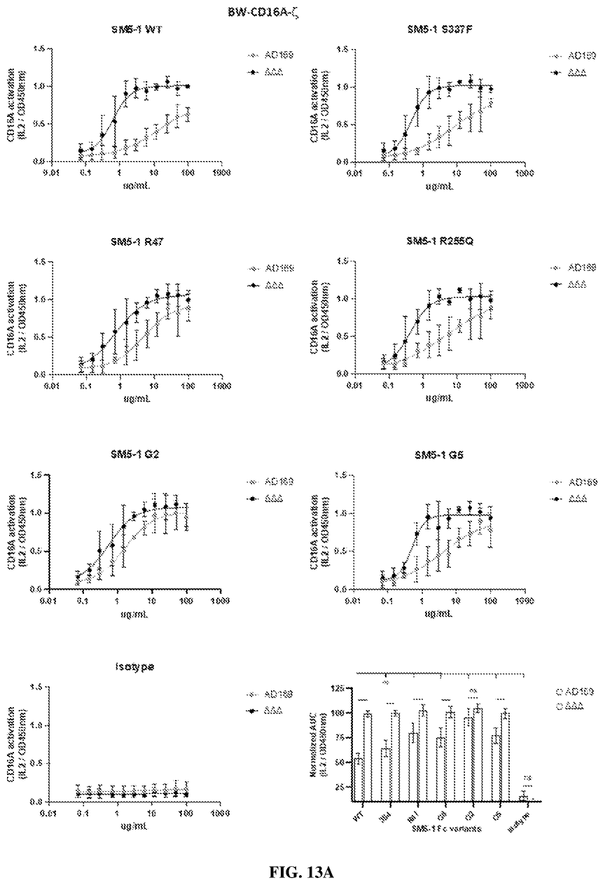

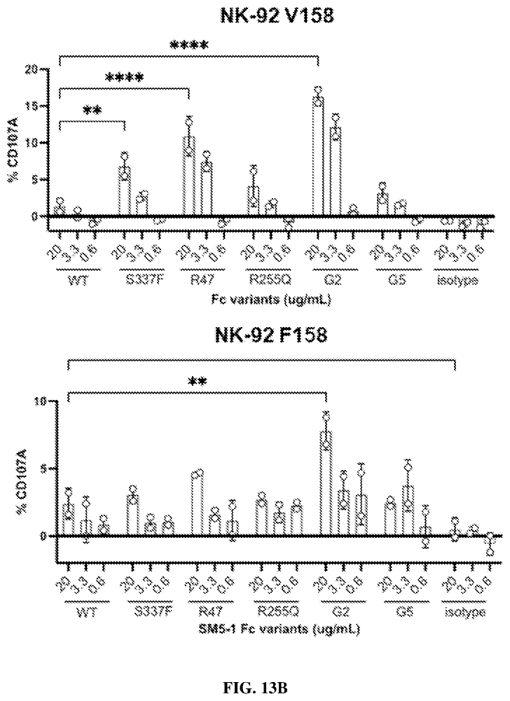

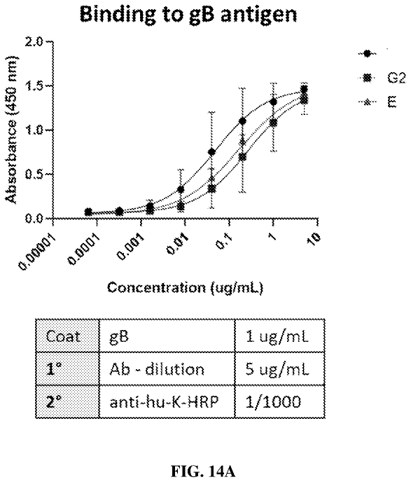

To assess binding in an avid cellular context, the vFcγR ectodomains were expressed on the CHO cell surface and binding of IgG1 Fc variants evaluated by flow cytometry. While the RL12 and RL13 sequences vary in clinical strains, conserved homologies (Cortese et al., 2012) allowed for identification and cloning of their putative Ig fold domains (FIG. 11A). Expression of gp34, gp68, Merlin gpRL12, AD169 gpRL12 and Merlin gpRL13 ectodomains with PDGFR anchors was confirmed by anti-FLAG and human IgG1 binding (FIG. 11B). Cells displaying Merlin gpRL12 and gpRL13 ectodomains showed reduced antibody binding in the presence of soluble gp34-M, suggesting these RL gene family members share overlapping epitopes with different fine specificities (FIG. 11B). Conversely, antibodies bearing the G2 and G5 Fcs show reduced binding to gp68 and gp34 compared to wild-type IgG1 (p<0.0001). Additionally, G2 exhibited lower binding to two gpRL12 isoforms (Merlin and AD169, p<0.0001) while G5 had reduced binding to gpRL13 (˜5 fold lower EC₅₀; p<0.0001) compared to wild-type IgG1 (FIGS. 3E,11C).

Comparison of the variant Fc binding profiles provides insight into the roles of individual residue changes (FIGS. 9A,D). Reduced binding of G5 to gpRL13 and gp34 can be attributed to the S337F mutation, whereas reduced binding of G2 to gpRL12 and gp34 can be attributed to the four residue changes in R47. Interestingly, gp34 binding seems to be impacted allosterically by S337F, which is located near the CH₂—CH₃ interface distal to the gp34 epitope at the Fc apex. Although the vFcγRs may manifest functions other than Fc binding, the presence of four proteins with similar Fc binding abilities but different binding specificities suggests that Fc capture is a key HCMV immune evasion strategy.

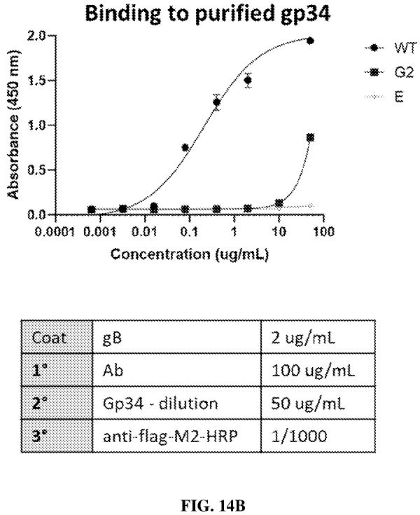

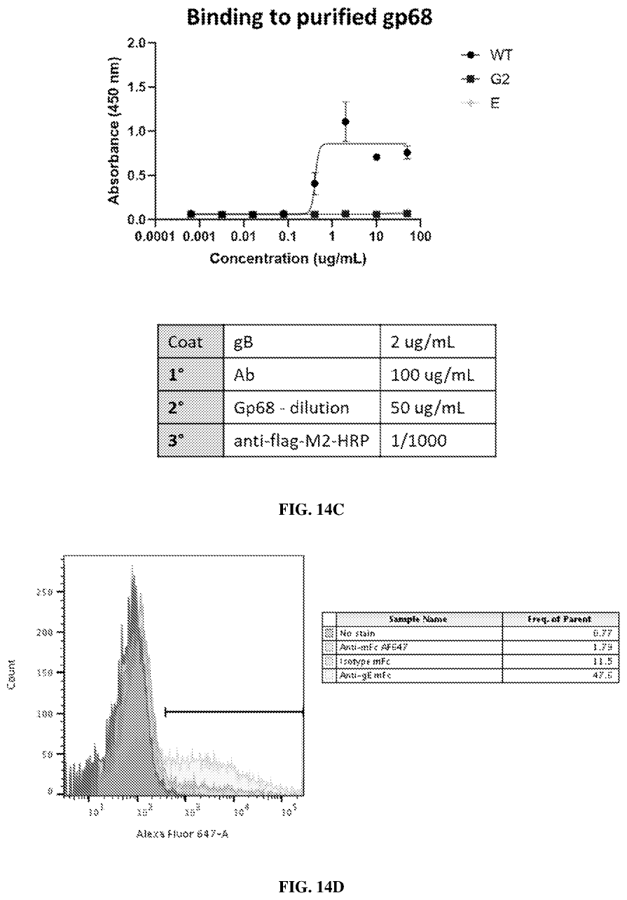

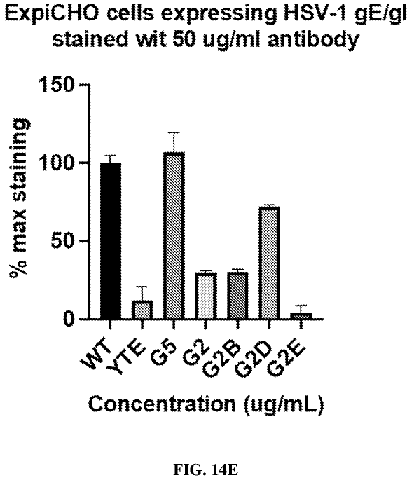

Fc variant G2 was further modified by reverting the E294K mutation to produce variant G2B. A further variant, G2E, was generated by adding the S337F mutation to G2B. These variants were cloned for expression as human IgG1 proteins SM5-1, binding gB. Binding of the G2E variants was evaluated by ELISA. As a control for antibody integrity, binding to plates coated with the gB antigen was first evaluated (FIG. 14A). Binding to the viral Fc receptors gp34 and gp68 was evaluated with ELISAs coated with either protein (FIGS. 14B-C). As with the Fc variant G2, the Fc variant G2E lost considerable binding to gp34 and gp68 as compared to WT Fc.

Binding of Fc variants to HSV-1 gE/gI viral Fc receptor expressed on ExpiCHO cells was also evaluated (FIGS. 14D-E). At high antibody concentration (50 μg/ml), antibodies with WT human IgG1 Fc and the G5 variant bind gE/gI similarly, thus the changes in G5 (R255Q, S337F) do not contribute to gE/gI escape. The YTE (M252Y/S254T/T256E) changes, which are used in several antibodies in clinical trials to extend circulating half-life, lose binding to gE/gI, but not HCMV gp68. As such, Fc variants identified here also lose binding to alpha-herpes virus receptors gE/gI. Specifically, all variants containing Fc residue change K334E (G2, G2E and G2B) lose binding to HSV-1 gE/gI in a flow assay; while variants lacking this change (G2D) retain gE/gI binding. Fc residue Q311 forms H-bonds with R322 of gE (PDB 2GJ7); thus the Q311L change is expected to contribute to the loss of gE/gI binding observed. Fc residue R255Q is proximal to gE residues S245 and N243 and may contribute to loss of binding. In addition, the residue changes in G2 and G2E are expected to work together to evade capture by viral Fc receptors while retaining binding to host Fc receptors.

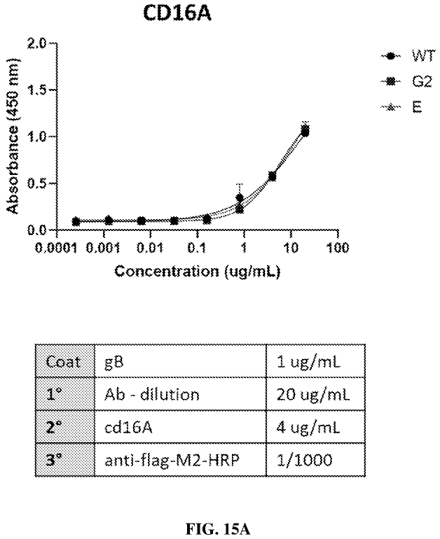

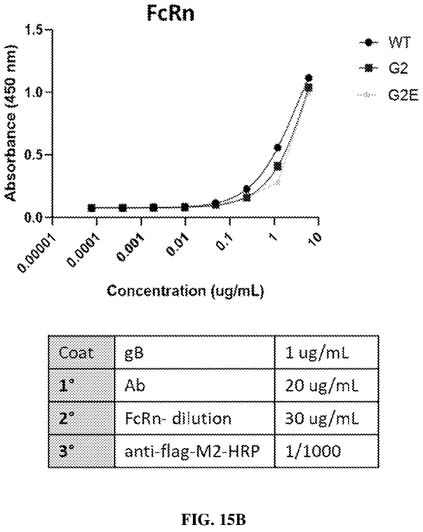

Binding to the host Fc receptors CD16a and FcRn was evaluated by performing an ELISA where the plate was coated with gB, then antibody serially diluted, then purified receptor added, with detection by anti-FLAG M2-HRP. Fc variants G2 and G2E retain binding to the host Fc receptors CD16a and FcRn as compared to WT Fc (FIGS. 15A-B).

### Example 8—vFcγR-Resistant Fc Variants Retain their Parental IgG1 Effector Functions

Bio-layer interferometry (BLI) was used to characterize binding affinities for the high affinity V158 and low affinity F158 human CD16A allotypes using hu4D5 antibodies with wild-type, G2, and G5 Fc domains. Fc variant affinities for V158 were ˜130-200 nM while those for F158 were all ˜1 μM (FIG. 3D, Table 2), similar to prior reports (Lee et al., 2019). Additionally, Fc/FcRn binding was evaluated using BLI at pH 6.0, with all variants exhibiting 50-90 nM K_{D} (Table 2) (Neuber et al., 2014) and, importantly, observed no binding for any variant at pH 7.4 (Table 2), which negatively impacts pharmacokinetics.

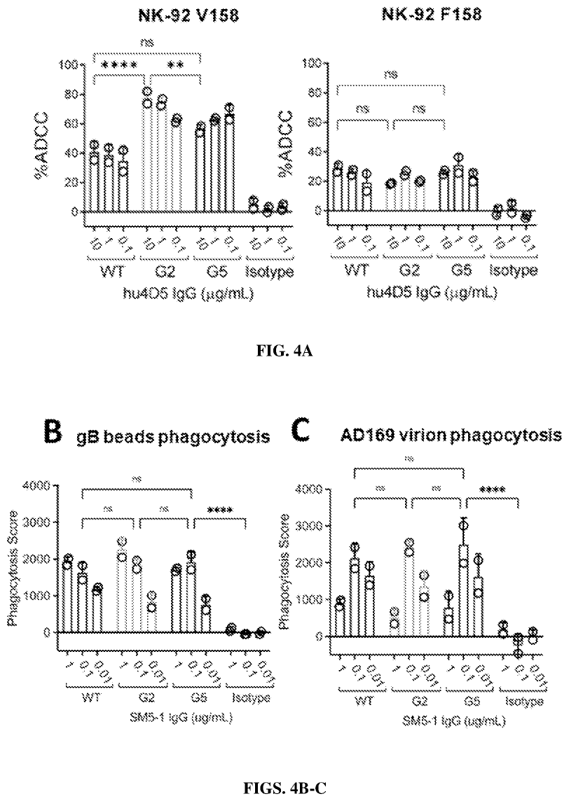

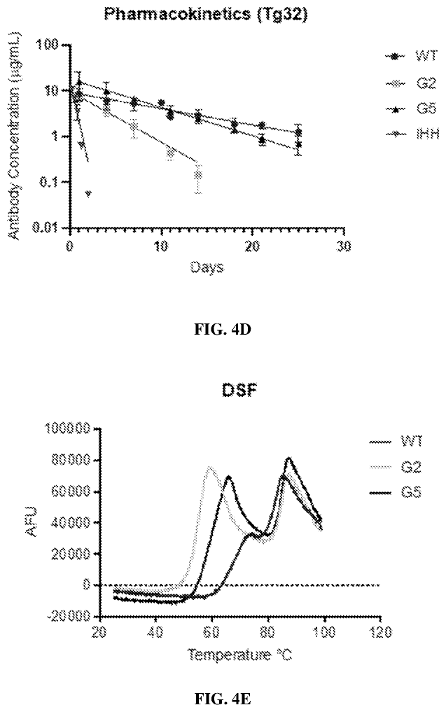

The antibodies were then evaluated for their abilities to mediate in vitro ADCC against HER2-expressing SKOV3 tumor cells. These experiments combined NK-92 effector cells stably expressing CD16A F158 or V158 alleles (Hsieh et al., 2017) with HER2-positive calcein labelled SKOV3 cells at 10:1 ratio for 4 hrs at 37° C. In the presence of antibodies with a wild-type Fc, NK-92 V158 mediated ˜30-40% target cell lysis, which increased to 60-80% for G2 or G5 Fc variants (FIGS. 4A,12A; p<0.001). In the presence of all Fc variants, NK-92 F158 cells mediated ˜20-30% target cell lysis ADCC.

To evaluate the effects of the G2 and G5 changes towards other activating FcγRs, SM5-1 antibodies were used in an ADCP assay, which plays a role in HCMV protection (Goodwin et al., 2020, Nelson et al., 2018). This assay used human THP-1 monocytes which express CD64 and CD32a, but not CD16a (Fleit and Kobasiuk, 1991) in the presence of pHrodo-labelled and gB-coated beads (50 beads per cell) or AD169 virions (2 per cell). Particle internalization was measured by flow cytometry, revealing similar phagocytosis scores for all Fc variants for both gB-coated beads and AD169 virions (FIGS. 4B,4C,12C,12D). Overall, the G2 and G5 Fc variants had minimal impacts on ADCP and ADCC induction in the absence of vFcγRs.

To provide a more rigorous assessment of FcRn binding behavior, antibody clearance rates were measured in homozygous Tg32 mice expressing human FcRn (Avery et al., 2016). After i.p. administration of 2 mg/kg antibody, the wild-type hu4D5 antibody exhibited a half-life of 8.8±0.9 days, similar to previous reports (Avery et al., 2016), while G5 was slightly reduced at 6.0±0.9 days (p=0.001) and G2 showed a significantly reduced half-life of 2.2±0.2 days (p<0.0001) (Table 3, FIG. 4D). The rapid G2 clearance was unexpected due to its unaltered FcRn binding (Table 2). Antibody thermal stability for wild-type hu4D5 showed a typical thermal melting curve, with the first isotherm, corresponding to the CH₂ melting temperature, occurring at 66.6±0.4° C. By contrast, G5 and G2 had lower Fc melting temperatures of 63±2 and 55.8±0.1° C., respectively (Table 3, FIG. 4E). This loss in thermal stability is not uncommon when engineering the CH₂ of an Fc and may explain the reduced in vivo half-life (Datta-Mannan, 2019).

TABLE 2
Binding affinities of Fc variants for host FcγRs measured by BLI.

| K_{D} (nM) | FcRn pH 6.0 | FcRn pH 7.4 | CD16A V158 | CD16A F158 |
|---|---|---|---|---|
| IgG1 | 60 ± 20 | ND | 200 ± 10 | 1000 ± 100 |
| G2 | 50 ± 10 | ND | 140 ± 30 | 1000 ± 600 |
| G5 | 90 ± 6 | ND | 180 ± 10 | 1000 ± 150 |

ND: not-determined kinetics due to low signal.

TABLE 3
Fc variant pharmacokinetics and thermostabilities.

| | Tm ° C. | t_{1/2} | Clearance |
|---|---|---|---|
| | (first transition) | (days) | (mL/day) |
| IgG1 | 67 ± 0.4 | 8.8 ± 0.9 | 0.30 ± 0.11 |
| G2E | cooperative | 8.5 ± 1.4 | ND |
| G2 | 56 ± 0.1** | 2.1 ± 0.2**** | 1.26 ± 0.37*** |
| G5 | 63 ± 2 | 6 ± 1*** | 0.43 ± 0.08 |
| IHH | ND | 0.22 ± 0.01**** | 4.30 ± 0.34**** |

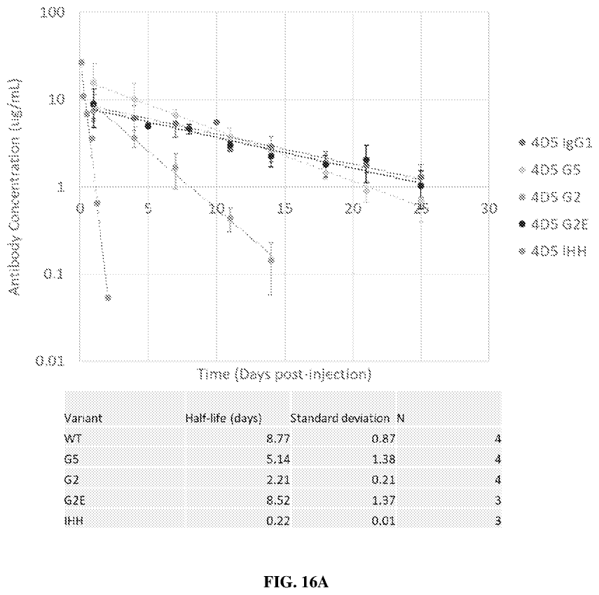

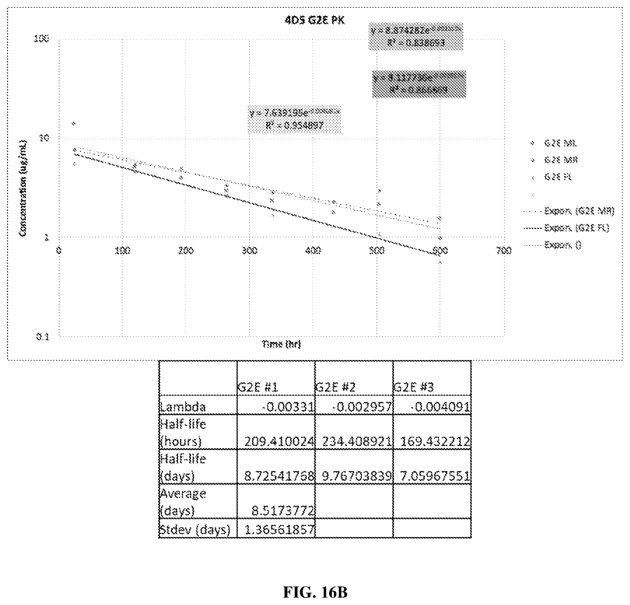

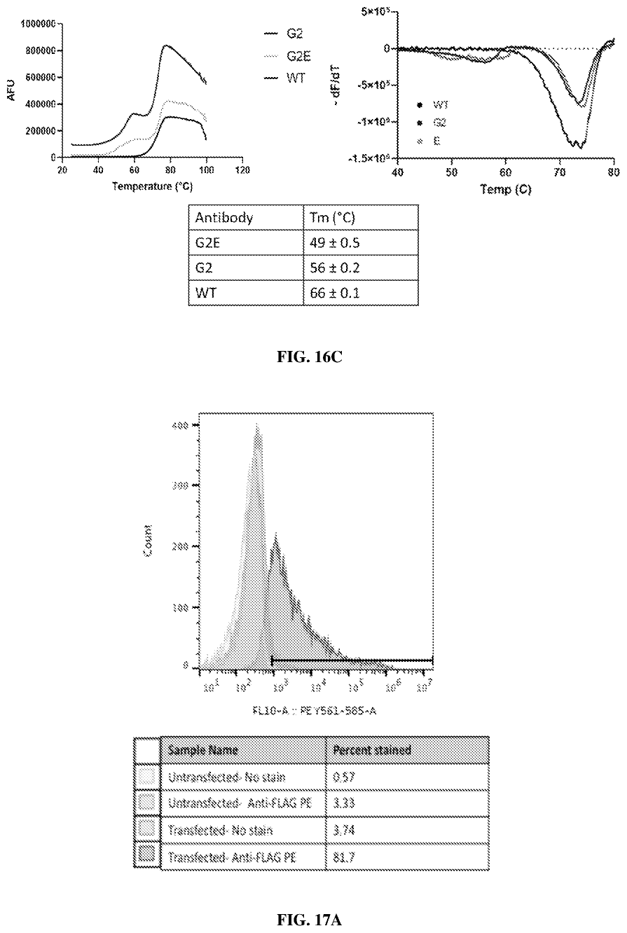

ND: not determined.
Data are mean ± SD and compared to WT-IgG1, one-way ANOVA was measured using GraphPad;
**p < 0.01,
***p < 0.001,
****p < 0.0001

Whereas antibody Fc variant G2 exhibits poor in vivo pharmacokinetics in humanized FcRn mice, G2E was found to have similar clearance kinetics as WT Fc. To demonstrate this, Tg32 homozygous mice expressing human FcRn under the human promoter were injected IP with 4D5 antibody with WT or engineered Fcs at 2 mg/kg. Serum was isolated from collected whole blood every 3-4 days post-injection. Antibody concentration in the serum was measured by ELISA as follows: HER2-Fc was coated onto ELISA plates and blocked using 5% milk in PBST. Coated wells were incubated with known serially diluted concentrations of 4D5 WT with 1:50 dilution of un-injected Tg32 mouse serum or injected mouse serum samples at 1:1000 to 1:50 dilutions at RT for 1 hr. A secondary anti-human kappa HRP antibody was applied at a concentration of 1:2000 and incubated at RT for 1 hr, followed by addition of TMB solution and neutralization with 1N HCl. As shown in FIG. 16A, G2E had similar clearance kinetics as WT Fc. Data for clearance of G2E from each replicate mouse is shown in FIG. 16B.

Antibody thermal stability for G2E was also evaluated. DSF was conducted on wild-type, G2, and G2E antibodies (FIG. 16C). The first transition likely represents CH₂ domain unfolding and is defined as the first thermal melting temperature (Tm) and used to compare antibodies since the value varies and the selected residue changes are in the CH₂ domain. The second transition is likely representative of CH₂ and CH₃ domain unfolding. The first transition is not present in WT and changes gradually in G2E, suggesting cooperative unfolding.

### Example 9—vFcγR-Resistant Fcs Mediate Potent Anti-Viral Responses Against CMV-Infected Cells

The ability of SM5-1 antibodies with G2 and G5 Fcs to resist internalization by AD169-infected MRC5 cells was evaluated (FIGS. 1C-D). Staining of cells by extracellular antibody was similar for all variants due to gB binding (˜70%, FIG. 5A). However, whereas ˜35% of the SM5-1 wild-type Fc was internalized, just 25% of G5 and 18% of G2-bearing antibodies were internalized (p<0.01; FIG. 5B), indicating vFcγR-resistance reduces clearance of anti-gB antibodies from the surface of infected cells.

The BW-CD16A-ζ reporter assay was used to assess immune activation following SM5-1 antibody engagement of AD169- or ΔΔΔ-infected MRC5 cells. All Fc variants showed similar abilities to mediate CD16A activation against ΔΔΔ-infected cells, measured as the EC₅₀ and the normalized area under the curve (AUC). However, wild-type IgG1 showed large differences when incubated with AD169- versus ΔΔΔ-infected cells (EC₅₀ values (95% CI) of 12.8 (5-33) vs 0.63 (0.4-0.9) pg/ml; p<0.0001). The differences between the two strains was reduced for the G5 Fc (EC₅₀ values (95% CI) of 4.0 (1.3-180) vs 0.63 (0.4-0.8) pg/ml; p<0.001) and even more reduced for the G2 Fc (EC₅₀ values (95% CI) of 1.7 (0.9-3) vs 0.6 μg/ml (0.3-1); p<0.01; FIG. 5C). P values indicate differences in EC50 for mAb's activation in the presence of AD169 and ΔΔΔ.

Finally, NK cell activation was measured by degranulation using NK-92 V158 and MRC5 fibroblasts infected with AD169/GFP. The wild-type SM5-1 triggered minimal CD107a upregulation (˜5% cells) at high antibody concentrations (20 μg/ml), similar to the isotype control (FIG. 5D). By contrast, the G2 Fc mediated significantly greater degranulation (˜17%, p<0.0001; FIG. 5D). Similar trends at higher G2 concentrations were observed for NK-92 cells expressing the low affinity F158 allele (FIG. 5E). Antibodies with the G5 Fc showed minimal if any increased degranulation (FIGS. 5D,E), likely reflecting the 2-fold stronger G5 affinity for gp68 and 10-fold stronger G5 affinity for gp34 than G2 (Table 1).

### Example 10—Anti-gp34 Antibodies

The viral Fc receptor gp34 is perfectly conserved across strains and highly expressed on infected cells. As such, it may serve as a good vaccine antigen to elicit antibodies that eliminate CMV-infected cells, and antibodies binding gp34 may have potential as therapeutic monoclonal antibody when combined with the G2E Fc. Indeed, the successful Shingrix vaccine is comprised only to the homologous protein from varicella zoster virus. Provided herein in Tables 4-6 are the sequences of three antibodies binding unique epitopes on gp34 with high affinity.

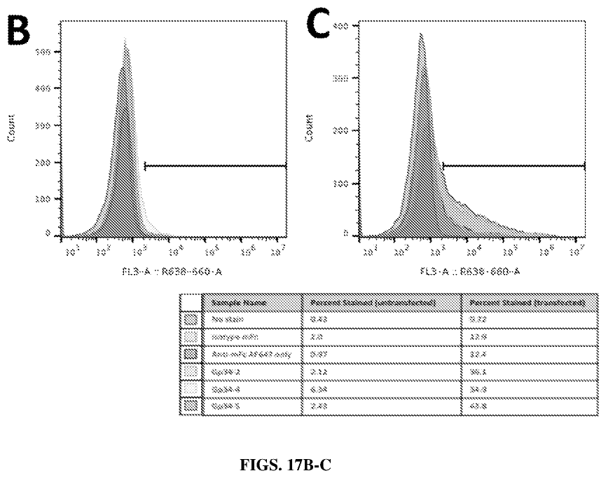

Binding to gp34 by these antibodies was confirmed by staining in a flow cytometry assay (FIGS. 17A-C). The variable light and heavy domains of these antibodies were cloned into mammalian expression plasmids with a human CL sequence and a human CH₁ region connected to murine IgG2a Fc (mFc), respectively. These antibodies were solubly expressed in ExpiCHO cells and purified using Protein A affinity chromatography. Then, hCMV gp34 was expressed on the surface of ExpiCHO cells using a mammalian expression plasmid containing a signal sequence, gp34 ectodomain with a C-terminal FLAG tag, and a PDGFR transmembrane domain. ExpiCHO cells were transfected using the gp34 transmembrane construct and cultured for two days at 37° C./8% CO₂. Successful expression of gp34 on the surface was detected by staining transfected and untransfected ExpiCHO cells with a 1:500 dilution of anti-FLAG PE antibody for 1 hour on ice (Agilent Technologies, Catalogue #PJ315-1). Binding of the anti-gp34 antibodies was detected by staining gp34-transfected and untransfected cells with each antibody-mFc at 10 ug/mL (6.7 nM) for 1 hour on ice followed by staining with a 1:500 dilution of anti-mouse Fcγ AlexaFluor 647 (Jackson ImmunoResearch Catalogue #115-605-071) for 1 hour on ice. Fluorescence was detected using a BD Fortessa system.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
AICHELER et al., 2013. Potential for Natural Killer Cell-Mediated Antibody-Dependent Cellular Cytotoxicity for Control of Human Cytomegalovirus. Antibodies, 2, 617-635.
ATALAY et al., 2002. Identification and Expression of Human Cytomegalovirus Transcription Units Coding for Two Distinct Fc& #x3b3; Receptor Homologs. Journal of Virology, 76, 8596-8608.
AVERY et al., 2016. Utility of a human FcRn transgenic mouse model in drug discovery for early assessment and prediction of human pharmacokinetics of monoclonal antibodies. MAbs, 8, 1064-78.
BERNSTEIN et al., 2016. Safety and efficacy of a cytomegalovirus glycoprotein B (gB) vaccine in adolescent girls: A randomized clinical trial. Vaccine, 34, 313-9.
BODER & WITTRUP, 2000. Yeast surface display for directed evolution of protein expression, affinity, and stability. Methods Enzymol, 328, 430-44.
BOESCH et al., 2017. IgG Fc variant cross-reactivity between human and rhesus macaque FcγRs. MAbs, 9, 455-465.
BORROK et al., 2017. An “Fc-Silenced” IgG1 Format With Extended Half-Life Designed for Improved Stability. J Pharm Sci, 106, 1008-1017.
BREY et al., 2018. A gB/CD3 bispecific BiTE antibody construct for targeting Human Cytomegalovirus-infected cells. Scientific Reports, 8, 17453.
BRITT, 2017. Congenital Human Cytomegalovirus Infection and the Enigma of Maternal Immunity. J Virol, 91.
CHAO et al., 2006. Isolating and engineering human antibodies using yeast surface display. Nat Protoc, 1, 755-68.
CHEN et al., 2017. Engineering Aglycosylated IgG Variants with Wild-Type or Improved Binding Affinity to Human Fc Gamma RIIA and Fc Gamma RIIIAs. J Mol Biol, 429, 2528-2541.
CIRINO et al., 2003. Generating mutant libraries using error-prone PCR. Methods Mol Biol, 231, 3-9.
CORRALES-AGUILAR et al., 2014a. CMV-encoded Fcγ receptors: modulators at the interface of innate and adaptive immunity. Seminars in Immunopathology, 36, 627-640.
CORRALES-AGUILAR et al., 2014b. Human Cytomegalovirus Fcγ Binding Proteins gp34 and gp68 Antagonize Fcγ Receptors I, II and III. PLOS Pathogens, 10, e1004131.
CORRALES-AGUILAR et al., 2013. A novel assay for detecting virus-specific antibodies triggering activation of Fcγ receptors. Journal of Immunological Methods, 387, 21-35.
CORTESE et al., 2012. Recombinant Human Cytomegalovirus (HCMV) RL13 Binds Human Immunoglobulin G Fc. PLOS ONE, 7, e50166.
COSTA-GARCIA et al., 2015. Antibody-mediated response of NKG2Cbright NK cells against human cytomegalovirus. J Immunol, 194, 2715-24.
DALL'ACQUA et al., 2006. Properties of human IgG1s engineered for enhanced binding to the neonatal Fc receptor (FcRn). J Biol Chem, 281, 23514-24.
DATTA-MANNAN, 2019. Mechanisms Influencing the Pharmacokinetics and Disposition of Monoclonal Antibodies and Peptides. Drug Metab Dispos, 47, 1100-1110.
DILILLO et al., 2016. Broadly neutralizing anti-influenza antibodies require Fc receptor engagement for in vivo protection. J Clin Invest, 126, 605-10.
FALK et al., 2018. Large-Scale Screening of HCMV-Seropositive Blood Donors Indicates that HCMV Effectively Escapes from Antibodies by Cell-Associated Spread. Viruses, 10.
FLEIT & KOBASIUK, 1991. The Human Monocyte-Like Cell Line THP-1 Expresses FcγRI and FCγRII. Journal of Leukocyte Biology, 49, 556-565.
FORREST et al., 2020. NK Cell Memory to Cytomegalovirus: Implications for Vaccine Development. Vaccines (Basel), 8.
GOODWIN et al., 2020. Specificity and effector functions of non-neutralizing gB-specific monoclonal antibodies isolated from healthy individuals with human cytomegalovirus infection. Virology, 548, 182-191.
GRANT et al., 2018. cisTEM, user-friendly software for single-particle image processing. eLife, 7, e35383.
GRIFFITHS et al., 2011. Cytomegalovirus glycoprotein-B vaccine with MF59 adjuvant in transplant recipients: a phase 2 randomised placebo-controlled trial. Lancet, 377, 1256-63.
GUNN et al., 2021. A Fc engineering approach to define functional humoral correlates of immunity against Ebola virus. Immunity, 54, 815-828.e5.
HE et al., 2016. Epitope specificity plays a critical role in regulating antibody-dependent cell-mediated cytotoxicity against influenza A virus. Proc Natl Acad Sci USA, 113, 11931-11936.
HEINEMAN et al., 2019. Understanding the immunology of Shingrix, a recombinant glycoprotein E adjuvanted herpes zoster vaccine. Curr Opin Immunol, 59, 42-48.
HORWITZ et al., 2017. Non-neutralizing Antibodies Alter the Course of HIV-1 Infection In Vivo. Cell, 170, 637-648.e10.
HSIEH et al., 2017. Characterization of FcγRIIIA effector cells used in in vitro ADCC bioassay: Comparison of primary NK cells with engineered NK-92 and Jurkat T cells. J Immunol Methods, 441, 56-66.
JENKS et al., 2019. The Roles of Host and Viral Antibody Fc Receptors in Herpes Simplex Virus (HSV) and Human Cytomegalovirus (HCMV) Infections and Immunity. Frontiers in Immunology, 10.
JENKS et al., 2020. Antibody binding to native cytomegalovirus glycoprotein B predicts efficacy of the gB/MF59 vaccine in humans. Science Translational Medicine, 12, eabb3611.
KARSTENTISCHER et al., 2006. Two-step Red-mediated recombination for versatile high-efficiency markerless DNA manipulation in Escherichia coli. BioTechniques, 40, 191-197.
KOLB et al., 2021. Human cytomegalovirus antagonizes activation of Fcγ receptors by distinct and synergizing modes of IgG manipulation. Elife, 10.
KOLB et al., 2019. Identification and Functional Characterization of a Novel Fc Gamma-Binding Glycoprotein in Rhesus Cytomegalovirus. J Virol, 93.
LAZAR et al., 2006. Engineered antibody Fc variants with enhanced effector function. Proc Natl Acad Sci USA, 103, 4005-10.
LE et al., 2011. The Cytomegaloviral Protein pUL138 Acts as Potentiator of Tumor Necrosis Factor (TNF) Receptor 1 Surface Density To Enhance UL<i>b</i>&#x2032;—Encoded Modulation of TNF-& #x3b1; Signaling. Journal of Virology, 85, 13260-13270.
LE-TRILLING et al., 2020. The Human Cytomegalovirus pUL145 Isoforms Act as Viral DDB1-Cullin-Associated Factors to Instruct Host Protein Degradation to Impede Innate Immunity. Cell Reports, 30, 2248-2260.e5.
LEE et al., 2019. An engineered human Fc domain that behaves like a pH-toggle switch for ultra-long circulation persistence. Nature Communications, 10, 5031.
LI et al., 2017. Complement enhances in vitro neutralizing potency of antibodies to human cytomegalovirus glycoprotein B (gB) and immune sera induced by gB/MF59 vaccination. npj Vaccines, 2, 36.
LIU et al., 2021. Prefusion structure of human cytomegalovirus glycoprotein B and structural basis for membrane fusion. Science Advances, 7, eabf3178.
MACCORMAC & GRUNDY, 1996. Human cytomegalovirus induces an Fc gamma receptor (Fc gammaR) in endothelial cells and fibroblasts that is distinct from the human cellular Fc gammaRs. J Infect Dis, 174, 1151-61.
MINTON, 2007. Static light scattering from concentrated protein solutions, I: General theory for protein mixtures and application to self-associating proteins. Biophysical journal, 93, 1321-1328.
MODLIN et al., 2004. Vaccine Development to Prevent Cytomegalovirus Disease: Report from the National Vaccine Advisory Committee. Clinical Infectious Diseases, 39, 233-239.
NELSON et al., 2018. HCMV glycoprotein B subunit vaccine efficacy mediated by nonneutralizing antibody effector functions. Proceedings of the National Academy of Sciences, 115, 6267-6272.
NEUBER et al., 2014. Characterization and screening of IgG binding to the neonatal Fc receptor. mAbs, 6, 928-942.
NGUYEN et al., 2018. Identification of high affinity HER2 binding antibodies using CHO Fab surface display. Protein engineering, design & selection: PEDS, 31, 91-101.
NIGRO et al., 2005. Passive immunization during pregnancy for congenital cytomegalovirus infection. N Engl J Med, 353, 1350-62.
PASS et al., 2009. Vaccine prevention of maternal cytomegalovirus infection. N Engl J Med, 360, 1191-9.
PATEL et al., 2018. HCMV-Encoded NK Modulators: Lessons From in vitro and in vivo Genetic Variation. Frontiers in Immunology, 9.
PLOTKIN, 2015. The history of vaccination against cytomegalovirus. Med Microbiol Immunol, 204, 247-54.
PROFF et al., 2018. Turning the tables on cytomegalovirus: targeting viral Fc receptors by CARs containing mutated CH2-CH3 IgG spacer domains. J Transl Med, 16, 26.
PUNJANI et al., 2017. cryoSPARC: algorithms for rapid unsupervised cryo-EM structure determination. Nature Methods, 14, 290-296.
REVELLO et al., 2014. A randomized trial of hyperimmune globulin to prevent congenital cytomegalovirus. N Engl J Med, 370, 1316-26.
ROSSIGNOL et al., 2021. Mining HIV controllers for broad and functional antibodies to recognize and eliminate HIV-infected cells. Cell Reports, 35, 109167.
SCHOPPEL et al., 1996. Antibodies specific for the antigenic domain 1 of glycoprotein B (gpUL55) of human cytomegalovirus bind to different substructures. Virology, 216, 133-45.
SPRAGUE et al., 2008. The human cytomegalovirus Fc receptor gp68 binds the Fc CH2-CH3 interface of immunoglobulin G. J Virol, 82, 3490-9.
VAN DEN HOECKE et al., 2017. Hierarchical and Redundant Roles of Activating FcγRs in Protection against Influenza Disease by M2e-Specific IgG1 and IgG2a Antibodies. J Virol, 91.
VIETZEN et al., 2017. Association between antibody functions and human cytomegalovirus (HCMV) replication after lung transplantation in HCMV-seropositive patients. J Heart Lung Transplant.
VIETZEN et al., 2020. Human Cytomegalovirus (HCMV)-Specific Antibody Response and Development of Antibody-Dependent Cellular Cytotoxicity Against HCMV After Lung Transplantation. The Journal of Infectious Diseases, 222, 417-427.
VLAHAVA et al., 2021. Monoclonal antibodies targeting nonstructural viral antigens can activate ADCC against human cytomegalovirus. The Journal of Clinical Investigation, 131.
WAGNER et al., 2002. Herpesvirus genetics has come of age. Trends in Microbiology, 10, 318-324.
WANG et al., 2018. IgG Fc engineering to modulate antibody effector functions. Protein Cell, 9, 63-73.
WU et al., 2015a. Natural killer cells can inhibit the transmission of human cytomegalovirus in cell culture by using mechanisms from innate and adaptive immune responses. J Virol, 89, 2906-17.
WU et al., 2015b. Natural killer cells can inhibit the transmission of human cytomegalovirus in cell culture by using mechanisms from innate and adaptive immune responses. Journal of virology, 89, 2906-2917.

1. A recombinant protein comprising
an engineered human IgG1 Fc domain having reduced affinity for a viral Fc receptor as compared to a wild-type human IgG1 Fc domain, wherein the engineered human IgG1 Fc domain comprises substitution at one of the following set of positions: (a) R255, H268, E294, Q311, K334, and S337; (b) R255, H268, Q311, and K334; (c) R255, H268, Q311, K334, and S337; (d) R255, H268, E294, Q311, and K334; or (e) R255 and S337, wherein the positions are numbered according to the EU numbering system.

2. The recombinant protein of claim 1, wherein the viral Fc receptor is a herpes viral Fc receptor.

3-10. (canceled)

11. The recombinant protein of claim 1, wherein the engineered human IgG1 Fc domain comprises the following substitution(s): R255Q, H268L, Q311L, and/or K334E, wherein the positions are numbered according to the EU numbering system.

12. The recombinant protein of claim 11, wherein the engineered human IgG1 Fc domain comprises an amino acid sequence of SEQ ID NO: 28 or 29.

13. (canceled)

14. The recombinant protein of claim 1, wherein the engineered human IgG1 Fc domain comprises the following substitution(s): R255Q, H268L, Q311L, K334E, and/or S337F, wherein the positions are numbered according to the EU numbering system.

15. The recombinant protein of claim 14, wherein the engineered human IgG1 Fc domain comprises an amino acid sequence of SEQ ID NO: 30 or 31.

16. (canceled)

17. The recombinant protein of claim 1, wherein the engineered human IgG1 Fc domain comprises the following substitution(s): R255Q, H268L, E294K, Q311L, and/or K334E, wherein the positions are numbered according to the EU numbering system.

18. The recombinant protein of claim 17, wherein the engineered human IgG1 Fc domain comprises an amino acid sequence of SEQ ID NO: 4 or 6.

19-21. (canceled)

22. The recombinant protein of claim 1, wherein the engineered human IgG1 Fc domain comprises the following substitution(s): R255Q and/or S337F, wherein the positions are numbered according to the EU numbering system.

23. The recombinant protein of claim 22, wherein the engineered human IgG1 Fc domain comprises an amino acid sequence of SEQ ID NO: 5 or 7.

24-26. (canceled)

27. The recombinant protein of claim 1, comprising at least one further substitution to increase an Fc effector function, wherein the at least one further substitutions comprises G236A, S239D, A330L, and/or I332E, wherein the positions are numbered according to the EU numbering system.

28-31. (canceled)

32. The recombinant protein of claim 1, comprising at least one further set of substitutions that alters affinity for FcRn, wherein the at least one further set of substitutions comprises (a) M252Y, S254T, and T256E: (b) L309D, Q311H, and N434S; or (c) M428L and N434S wherein the positions are numbered according to the EU numbering system.

33-35. (canceled)

36. The recombinant protein of any one of claims 1-35, wherein the recombinant protein is glycosylated.

37-41. (canceled)

42. The recombinant protein of claim 40 or 41, further comprising a variable domain that selectively binds to an immunogenic viral antigen, wherein the variable domain comprises clone-paired heavy and light chain CDR sequences derived from the clone-paired heavy chain and light chain variable sequences of Table 6.

43-53. (canceled)

54. An isolated nucleic acid encoding the recombinant protein of claim 1.

55-56. (canceled)

57. A hybridoma or engineered cell comprising a nucleic acid encoding the recombinant protein of claim 1.

58-59. (canceled)

60. A pharmaceutical formulation comprising one or more recombinant protein of claim 1.

61. A method of treating a subject comprising administering an effective amount of the pharmaceutical formulation of claim 60 to the subject.

62-70. (canceled)

71. An engineered protein comprising an engineered HCMV gp34 protein ectodomain that comprises a C150S substitution, with the position being relative to SEQ ID NO: 11.

72-85. (canceled)

86. A monoclonal antibody or antibody fragment, wherein the antibody or antibody fragment comprises clone-paired heavy and light chain CDR sequences derived from the clone-paired heavy chain and light chain variable sequences of Table 6.

87-105. (canceled)
